(19) United States
(12) Patent Application Publication
(10) Pub. No.: US 2004/0014168 A1
(43) Pub. Date: Jan. 22, 2004

(54) Reagents and methods for library synthesis and screening

(75) Inventors: Stuart L. Schreiber, Boston, MA (US); Helen E. Blackwell, Middleton, WI (US); Lucy Perez Fernandez, Cambridge, MA (US); John A. Tallarico, Boston, MA (US)

Correspondence Address: Choate, Hall & Stewart, Exchange Place, 53 State Street, Boston, MA, 02109, US

(73) Assignee: Howard Hughes Medical Institute

(21) Appl. No.: 10/243,974

(22) Filed: Sep. 13, 2002

Related U.S. Application Data
(63) Continuation-in-part of application No. 09/863,141, filed on May 22, 2001.
Application No. 09/863,141 is a continuation-in-part of application No. 09/838,760, filed on Apr. 19, 2001, now abandoned.
Application No. 09/838,760 is a continuation of application No. 09/329,970, filed on Jun. 10, 1999, now abandoned.
(60) Provisional application No. 60/089,124, filed on Jun. 11, 1998.

(51) Int. Cl. C12P 21/06; C08G 63/48; C08G 63/91

(52) U.S. Cl. 435/68.1; 525/54.1; 525/54.2

(57) ABSTRACT

The invention provides novel solid supports for the synthesis of compound libraries. The solid supports have the physical capacity to deliver at least 50 nmol of compound and are functionalized with a silicon-containing linker. In one embodiment of the invention the solid support comprises a bead having a diameter of between approximately 400 and 600 μm functionalized with a silicon-containing linker. According to certain embodiments of the invention the bead is a polystyrene bead. According to certain embodiments of the invention the linker is a diisopropylalkyl-substituted silyl ether. The invention further provides grafted polymeric supports such as lanterns functionalized with a silicon-containing linker.

The invention provides methods for screening in which compounds are synthesized on solid supports in a quantity so that a single solid support such as a bead provides a stock solution sufficient to perform hundreds or thousands of biological or chemical assays. The methods include decoding the sequence of chemical reactions used to synthesize the compound by identifying tags incorporated into the compound during synthesis.

### PRIORITY INFORMATION

The present application is a continuation-in-part application of U.S. patent application Ser. No. 09/863,141, filed May 22, 2001, entitled “Novel Alkaloids”, which is a continuation-in-part of Ser. No. 09/838,760, filed Apr. 19, 2001, entitled “Biomimetic Combinatorial Synthesis”, which application is a continuation application of U.S. patent application Ser. No. 09/329,970, filed Jun. 10, 1999, entitled “Biomimetic Combinatorial Synthesis”, now abandoned, which application claims priority to provisional application 60/089,124, filed Jun. 11, 1998, entitled “Biomimetic Combinatorial Synthesis”. The entire contents of each of these applications are hereby incorporated by reference in their entirety.

### GOVERNMENT SUPPORT

The United States Government has provided grant support utilized in the development of the present invention. In particular, this work was supported by National Institute of General Medical Sciences GM-52067. The United States Government may have certain rights in the invention.

### BACKGROUND OF THE INVENTION

The identification of small organic molecules that affect specific biological functions is an endeavor that impacts both biology and medicine. Such molecules are useful as therapeutic agents and as probes of biological function. In but one example from the emerging field of chemical genetics, in which small molecules can be used to alter the function of biological molecules to which they bind, these molecules have been useful at elucidating signal transduction pathways by acting as chemical protein knockouts, thereby causing a loss of protein function. (Schreiber et al., J. Am. Chem. Soc., 1990, 112, 5583; Mitchison, Chem. and Biol., 1994, 1, 3) Additionally, due to the interaction of these small molecules with particular biological targets and their ability to affect specific biological function, they may also serve as candidates for the development of therapeutics. One important class of small molecules, natural products, which are small molecules obtained from nature, clearly have played an important role in the development of biology and medicine, serving as pharmaceutical leads, drugs (Newman et al., Nat. Prod. Rep. 2000, 17, 215-234), and powerful reagents for studying cell biology (Schreiber, S. L. Chem. and Eng. News 1992 (October 26), 22-32).

Because it is difficult to predict which small molecules will interact with a biological target, intense efforts have been directed towards the generation of large numbers, or libraries, of small organic compounds. These libraries can then be linked to sensitive screens to identify the active molecules. Of particular interest has been the development of libraries based upon existing natural products. To date, however, libraries based on natural products have been synthesized primarily for the purpose of improving the known biological and pharmacokinetic properties of the parent natural products (Hall, D. G.; Manku, S.; Wang, F. J. Comb. Chem. 2001, 3(2), 125-150; Nicolaou, K. C.; Vourloumis, D.; Li, T.; Pastor, J.; Winssinger, N.; He, Y.; Ninkovis, S.; Sarabia, F.; Vallberg, H.; Roschanger, F.; King, N. P.; Finlay, R. V.; Giannakakou, P.; Verdier-Pinard, P.; Hamel, E. Angew. Chem., Int. Ed. Engl. 1997, 36, 2097-2103; Nicolaou, K. C.; Winssinger, D.; Vourloumis, D.; Ohshima, T.; Kim, S.; Pfefferkom, J.; Xu, J.-Y.; Li, T. J. Am. Chem. Soc. 1998, 120, 10814-10826; Lee, K. J.; Angulo, A.; Ghazal, P.; Janda, K. D. Org. Lett. 1999, 1, 1859-1862; Xu, R.; Greiveldinger, G.; Marenus, L. E.; Cooper, A.; Ellman, J. A. J. Am. Chem. Soc. 1999,121, 4898-4899; Wipf, P.; Reeves, J. T.; Balachandran, R.; Giuliano, K. A.; Hamel, E.; Day, B. W. J. Am. Chem. Soc. 2000, 122, 9391-9395; Boger, D. L.; Fink, B. E.; Hedrick, M. P. J. Am. Chem. Soc. 2000, 122, 6382-6394; Nicolaou, K. C.; Pfefferkom, J. A.; Barluenga, S.; Mitchell, H. J.; Roecker, A. J.; Cao, G.-Q. J. Am. Chem. Soc. 2000, 122, 9968-9976 and references cited therein).

Clearly, as detailed above, a great deal of research has been conducted to optimize existing natural product leads, the development of compounds and libraries of compounds based upon natural products and/or emulating the structural and stereochemical diversity of natural products, but having different biological activities than the parent natural product, would also be useful. Additionally, the development of novel synthetic methodologies would assist in the development of new classes of complex compounds and libraries of compounds. In order achieve greater diversity and complexity in the synthesis of compounds and particularly libraries of compounds, it would be desirable to develop such methods by either utilizing or emulating the rapid and stereoselective pathways that nature uses in the synthesis of natural products for the efficient production of complex compounds and libraries of compounds. Any resultant novel complex compounds and libraries based on biomimetic pathways will certainly be useful in the quest to discover either non-natural compounds having the binding affinities and specific characteristics of natural products, themselves the products of genetic recombination and natural selection, or will be particularly useful in the quest to discover compounds based upon natural products that exhibit novel biological properties.

Various approaches to the synthesis of compound libraries have been explored. For example, diversity-oriented synthesis (Schreiber, S., Science, 2000, 287, 1964-1969), solid-phase purification (Merrifield, R., J. Am. Chem. Soc., 1963, 85, 2149-2154), and the split-pool strategy (Furka, A., et al., Int. J. Pept. Protein Res., 1991, 37, 487-493) have allowed for efficient synthesis of compound libraries (Dolle, R., J. Comb. Chem., 2000, 2, 383-433). However, in order to effectively harness this synthetic ability to perform biological and/or chemical screens, a number of additional issues must be addressed. For example, there exists a need in the art for reagents and methods that would allow improved formatting of the compounds. In addition, there exists a need in the art for improved linkers for target compound synthesis and improved methods for encoding and decoding compound libraries.

### SUMMARY OF THE INVENTION

In one aspect of the invention, novel solid supports for the synthesis of compound libraries are provided. The solid supports have the physical capacity to deliver at least 50 nmol of target compound and are functionalized with a silicon-containing linker. In one embodiment of the invention the solid support comprises a bead having a diameter of greater than 500 μm, e.g., between approximately 400 and 600 μm, functionalized with a silicon-containing linker. According to certain embodiments of the invention the bead is a polystyrene bead. According to certain embodiments of the invention the linker is a diisopropylalkyl-substituted silyl ether. According to certain embodiments of the invention the linker includes a leaving group that can be displaced by a substrate, or a moiety that can react with a substrate, thereby allowing attachment of the substrate to the solid support.

The invention further provides grafted polymeric supports functionalized with a silicon-containing linker. The grafted polymeric supports may include a polystyrene surface and may be in the form of lanterns. According to certain embodiments of the invention the linker is a diisopropylalkyl-substituted silyl ether.

According to certain embodiments of the invention the linker attached to the solid support has the following structure prior to addition of a substrate:

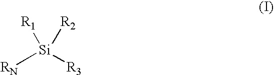

wherein R_{N}, is an aliphatic or heteroaliphatic moiety, wherein R_{N} is attached to the solid support; R₁ and R₂ are each independently hydrogen or an aliphatic, heteroaliphatic, aryl or heteroaryl moiety; and R₃ is an aliphatic, heteroaliphatic, aryl or heteroaryl moiety; halogen, —OSO₂Rₓ; wherein Rₓ is an aliphatic, heteroaliphatic, aryl or heteroaryl moiety; or O, S, —NR^{A} or -—CR^{A}R^{B}; wherein any two adjacent —CR^{A}R^{B} moieties may be linked by a single or double bond as valency permits; wherein each occurrence of R_{A} and R_{B} is independently absent, hydrogen, an aliphatic, heteroaliphatic, aryl or heteroaryl moiety, halogen, —CN, —S(O)ₘR^{J}, —NO₂, —COR^{J}, —CO₂R^{J}, —NR^{J}COR^{J}, —NR^{J}(CO)NR^{J}R^{J}, —NR^{J}CO₂R^{J}, —CONR^{J}R^{J}, —CO(NOR^{J})R^{J}, or —ZR^{J}, wherein Z is —O—, —S—, or NR^{K}; wherein each occurrence of R^{J} and R^{K} is independently hydrogen, —COR^{J}, —CO₂R^{J}, —CONR^{J}R^{J}, —CO(NOR^{J})R^{J}, or an aliphatic, heteroaliphatic, aryl or heteroaryl moiety, and m is 1 or 2; and wherein each of the foregoing aliphatic or heteroaliphatic moieties may be substituted or unsubstituted, cyclic or acyclic, linear or branched, saturated or unsaturated; and each of the foregoing aryl and heteroaryl moieties may be substituted or unsubstituted.

In certain embodiments of the invention, the linker has the following structure prior to addition of a substrate:

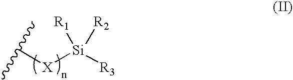

wherein R₁ and R₂ are each independently alkyl, heteroalkyl, aryl or heteroaryl; R₃ is aryl, heteroaryl, alkenyl, heteroalkenyl, halogen, or —OSO₂Rₓ; wherein Rₓ is an aliphatic, heteroaliphatic, aryl or heteroaryl moiety; X is O, S, —NR^{A} or —CR^{A}R^{B}; wherein any two adjacent —CR^{A}R^{B} moieties may be linked by a single or double bond as valency permits; wherein each occurrence of R_{A} and R_{B} is independently absent, hydrogen, an aliphatic, heteroaliphatic, aryl or heteroaryl moiety, halogen, —CN, —S(O)ₘR^{J}, —NO₂, —COR^{J}, —CO₂R^{J}, —NR^{J}COR^{J}, —NR^{J}(CO)NR^{J}R^{J}, —NR^{J}CO₂R^{J}, —CONR^{J}R^{J}, —CO(NOR^{J})R^{J}, or —ZR, wherein Z is —O—, —S—, or NR^{K}; wherein each occurrence of R^{J} and R^{K} is independently hydrogen, —COR^{J}, —CO₂R^{J}, —CONR^{J}R^{J}, —CO(NOR^{J})R^{J}, or an aliphatic, heteroaliphatic, aryl or heteroaryl moiety, and m is 1 or 2; and n is an integer from 1-10; wherein each of the foregoing alkyl, alkenyl, heteroalkenyl, heteroalkyl, aliphatic and heteroaliphatic moieties may be substituted or unsubstituted, cyclic or acyclic, linear or branched; and each of the foregoing aryl and heteroaryl moieties may be substituted or unsubstituted.

The invention further provides certain novel silicon-containing linkers themselves and also methods of synthesizing compound libraries using the inventive support/linker systems and provides compound libraries synthesized according to these methods.

The invention also provides methods of screening compound libraries for biological and/or chemical activity. The methods include small molecule printing and exposing a biological system such as a cell, tissue, organism, etc. to the compounds and observing a response. The invention provides a “one-bead one-stock solution” approach to library synthesis and screening in which sufficient compound is synthesized on an individual solid support such as a macrobead so that hundreds of biological or chemical assays may be performed on a stock solution generated by cleaving the compound from the bead. According to the invention a number of the steps may be performed robotically.

In another aspect, the invention provides methods of encoding and decoding compounds by analyzing tags incorporated into the compounds rather than into solid supports.

This application refers to various publications including books, patents and patent applications, patent publications, and journal articles. The contents of all such references are incorporated herein by reference.

Definitions

For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference. It will be appreciated as described below, that a variety of inventive compounds and linkers can be synthesized according to the methods described herein. The starting materials and reagents used in preparing these compounds are either available from commercial suppliers such as Aldrich Chemical Company (Milwaukee, Wis.), Bachem (Torrance, Calif.), Sigma (St. Louis, Mo.), or are prepared by methods well known to a person of ordinary skill in the art following procedures described in such references as Fieser and Fieser 1991, “Reagents for Organic Synthesis”, vols 1-17, John Wiley and Sons, New York, N.Y, 1991; Rodd 1989 “Chemistry of Carbon Compounds”, vols. 1-5 and supps, Elsevier Science Publishers, 1989; “Organic Reactions”, vols 1-40, John Wiley and Sons, New York, N.Y., 1991; March 2001, “Advanced Organic Chemistry”, 5th ed. John Wiley and Sons, New York, N.Y.; and Larock 1989, “Comprehensive Organic Transformations”, VCH Publishers. These schemes are merely illustrative of some methods by which the compounds and linkers of this invention can be synthesized, and various modifications to these schemes can be made and will be suggested to a person of ordinary skill in the art having regard to this disclosure.

Furthermore, it will be appreciated by one of ordinary skill in the art that the synthetic methods, as described herein, utilize a variety of protecting groups. By the term “protecting group”, has used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

It will be appreciated that compounds synthesized utilizing the inventive solid supports and methods as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used, herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example of proliferative disorders, cancer, wound healing, infectious diseases, and immunological diseases. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched), branched, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes both straight, branched and cyclic alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups.

In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 aliphatic carbon atoms. Illustrative aliphatic groups thus include, but are not limited to, for example, methyl, ethyl, n-propyl, isopropyl, cyclopropyl, —CH₂-cyclopropyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

The term “alkoxy”, or “thioalkyl” as used herein refers to an alkyl group, as previously defined, attached to the parent molecular moiety through an oxygen atom or through a sulfur atom. In certain embodiments, the alkyl group contains 1-20 alipahtic carbon atoms. In certain other embodiments, the alkyl group contains 1-10 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include but are not limited to, methoxy, ethoxy, propoxy, isopropoxy, n-butoxy, tert-butoxy, neopentoxy and n-hexoxy. Examples of thioalkyl include, but are not limited to, methylthio, ethylthio, propylthio, isopropylthio, n-butylthio, and the like.

The term “alkylamino” refers to a group having the structure —NHR′ wherein R′ is alkyl, as defined herein. In certain embodiments, the alkyl group contains 1-20 aliphatic carbon atoms. In certain other embodiments, the alkyl group contains 1-10 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, iso-propylamino and the like.

Some examples of substituents of the above-described aliphatic (and other) moieties of compounds of the invention include, but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples which are described herein.

In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl and the like. In certain embodiments of the present invention, the term “heteroaryl”, as used herein, refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups (including bicyclic aryl groups) can be unsubstituted or substituted, wherein substitution includes replacement of one, two or three of the hydrogen atoms thereon independently with any one or more of the following moieties including, but not limited to: aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic or hetercyclic moieties, may optionally be substituted with substituents including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

The term “heteroatom” as used herein, refers to an oxygen, sulfur, nitrogen, phosphorus, or silicon, “heteroaliphatic”, as used herein, refers to aliphatic moieties which contain one or more oxygen, sulfur, nitrogen, phosphorous or silicon atoms, e.g., in place of carbon atoms. Heteroaliphatic moieties may be branched, unbranched or cyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

The terms “halo” and “halogen” as used herein refer to an atom selected from fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “heterocycloalkyl” or “heterocycle”, as used herein, refers to a non-aromatic 5-, 6- or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one, two or three of the hydrogen atoms thereon with but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

The term “solid support”, as used herein, refers to a material having a rigid or semi-rigid surface. Such materials will generally take the form of small beads, pellets, disks, chips, dishes, gears, langterns, multi-well plates, glass slides, wafers, or the like, although other forms may be used. In some embodiments, at least one surface of the substrate will be substantially flat. The term “surface” refers to any generally two-dimensional structure on a solid substrate and may have steps, ridges, kinks, terraces, and the like without ceasing to be a surface. When present in the form of individual particles (e.g., beads), solid supports are also referred to herein as “resins”. This term may also be used to refer to the material from which the solid supports are formed.

The term “bead” refers to a substantially spherical solid support. It is to be understood that the beads need not be perfectly spherical. For example, they may be flattened spheres and/or may display surface irregularities. With respect to the beads employed in the present invention, a key feature is that they possess a large enough surface area to support functionalization with a sufficient number of linker molecules to allow synthesis of approximately 50 nmol of compound. Various three-dimensional geometries are acceptable and are encompassed within the invention. When referring to the diameter of a bead, in the case of a solid support that is not perfectly spherical, the diameter is that which would result in an equivalent volume if the solid support was perfectly spherical. Generally, unless otherwise evident from the context, the term “approximately” means that the measurement may deviate by 10% or less from the numeral given, and the ranges listed are assumed to include both endpoints.

The term “polymeric support”, as used herein, refers to a soluble or insoluble polymer to which an amino acid or other chemical moiety can be covalently bonded by reaction with a functional group of the polymeric support. Many suitable polymeric supports are known, and include soluble polymers such as polyethylene glycols or polyvinyl alcohols, as well as insoluble polymers such as polystyrene resins. A suitable polymeric support includes functional groups such as those described below. A polymeric support is termed “soluble” if a polymer, or a polymer-supported compound, is soluble under the conditions employed. However, in general, a soluble polymer can be rendered insoluble under defined conditions. Accordingly, a polymeric support can be soluble under certain conditions and insoluble under other conditions.

The term “solid-phase synthesis” as used herein has its art-accepted meaning and generally refers to a synthetic approach in which the compound or a precursor thereof is attached to a solid support during some or all of the synthetic steps.

The term “linker”, as used herein, refers to a chemical moiety utilized to attach a compound of interest to a solid support to facilitate synthesis of inventive compounds. Typically, though not necessarily, the moiety includes a leaving group that may be displaced by a substrate in order to link the substrate to the solid support. Depending on the context, the term “linker” may refer to the chemical moiety either prior to attachment of the substrate, in which case the term “linker” includes the leaving group or reactive moiety. The term “linker” may also refer to the portion of the chemical moiety remaining after attachment of the substrate, in which case the leaving group is no longer included in the linker.

The term “grafted polymeric surface” refers to a polymer which has been modified by graft polymerization. Derivatives, blends and copolymers thereof modified by graft polymerization are also within the scope of the invention. In general, grafting refers to a polymer reaction in which blocks of one or more species are connected as side chains to a polymer molecule. Suitable types of graft polymerization include gamma-irradiation graft polymerization, ozone-induced graft polymerization, plasma-induced graft polymerization, UV-initiated graft polymerization and chemical-initiated graft polymerization, such as peroxide-initiated graft polymerization. A solid support having a grafted polymeric surface is referred to herein as a “grafted polymeric support”.

The term “modular”, in reference to a grafted polymeric surface, means that the grafted polymeric surface is in the form of a plurality of physical units which are suitable for use in a set of simultaneous or sequential chemical reactions, and which provide reproducible chemical properties. These may be of a wide variety of desired shapes, such as lanterns, gears, pins, pucks, discs, beads, microtitre plates, sheets, etc. It will be appreciated that the grafted polymer may be molded into any shape, depending on the desired application. This also provides flexibility in the physiochemical properties of the grafted polymeric support, and means that a specialized containment apparatus is not required.

### DESCRIPTION OF THE DRAWING

FIG. 1 depicts an outline of an integrated method of compound library synthesis according to the “one-bead, one stock solution” approach.

FIG. 2 shows a general reaction protocol for encoding macrobeads with diazoketone tags. The chemical bonds broken upon compound cleavage and tag cleavage are highlighted in bold. PS=polystyrene, DVB=divinylbenzene, HF=hydrogen fluoride, CAN=ceric ammonium nitrate.

FIG. 3 shows an encoding test support used in compound decoding experiments, also referred to as test support 10 herein.

FIG. 4 shows characterization of the dummy compound cleaved from ˜20 mg of test support 10 before (a,b) and after (c,d) the encoding reaction by ¹H nuclear magnetic resonance (NMR) and tandem liquid chromatography/mass spectroscopy (LC/MS). The MS (APCl) spectra for the main peak in the LC traces corresponded to the molecular ion of the compound (M+H=236). No molecular ions or fragments corresponding to tag insertion products (or with diagnostic chlorine isotope splitting patterns) were observed in the MS spectrum of the model compound after the encoding reaction.

FIG. 5a shows the structure of decoding test support 12 encoded with four tags. FIG. 5b shows a schematic of the glass autosampler insert used as a tag cleavage vessel.

FIG. 6 shows an overview of certain aspects of library formatting and annotation screening according to certain embodiments of the invention.

FIG. 7 shows operation of a bead arrayer for arraying beads into individual wells of a microtiter plate.

FIG. 8 shows a graph of quantitative GC data for decoding either from an individual macrobead of the test support depicted in FIG. 3 or from 1 or 5% (black and dark gray respectively) compound stock solutions prepared from a single macrobead of the support of FIG. 3.

FIG. 9 shows three methods to determine the chemical history of a macrobead relying on encoding. Examples of representative GC traces for test support 12 are shown: (a) tags cleaved from one macrobead before compound cleavage, (b) tags cleaved from one macrobead after compound cleavage, and (c) tags cleaved from 100% of the compound stock solution (GC peaks are off-scale due to saturation of the GC/EC detector).

FIG. 10 shows a graph of the observed complementarity between the GC and MS decoding of 108 samples from the dihydropyrancarboxamide library.

FIG. 11 shows a robotic 384 pin arrayer that may be used to array compounds from stock solutions into 384-well plates.

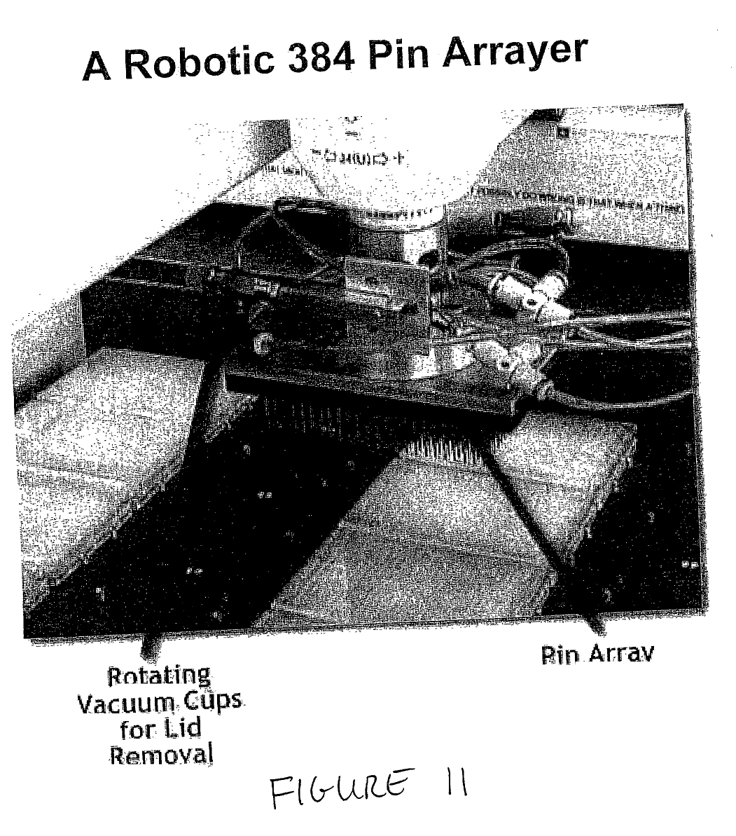

FIG. 12 depicts small molecule printing.

FIG. 13 depicts a small molecule microarraying robot.

FIG. 14 depicts a D series (FIG. 14a) and an L series (FIG. 14b) lantern.

FIG. 15a shows ten LC traces from the cleavage of 10 separate beads containing trans-2-phenyl-1 cyclohexanol. The large peak at 0.70 min. arises from residual pyridine and the solvent front of the injected solution. The average bead loading from this experiment is approximately 137 nmol/bead.

FIG. 15b shows ten LC traces from the cleavage of 10 separate beads containing 4-bromo-3,5-dimethylphenol. The large peak at 0.70 min. arises from residual pyridine and the solvent front of the injected solution. The average bead loading from this experiment is approximately 158 nmol/bead.

### DETAILED DESCRIPTION OF THE INVENTION

The ability to synthesize large and diverse libraries of small molecules opens up new possibilities for the identification of pharmaceutical lead compounds. In addition, screens of such libraries may be used to probe biological processes and to identify biological target molecules. For example, compounds can be screened to identify those capable of inducing particular biological phenotypes, responses, etc. Once a small molecule has demonstrated biological activity and the protein target identified, a role for that target within the biological system under study can be inferred. Compounds can also be screened for their ability to bind a preselected target (e.g., a protein or nucleic acid).

As mentioned above, one approach to the creation of compound libraries involves solid-phase synthesis, in which the compounds are synthesized on solid supports such as beads. Typically, such beads are derivatized with a linker molecule to which one or more substrates are attached. For example, as described in more detail in Example 3, a set of alcohols may be loaded onto the beads. The substrate serves as the initial building block for the compounds. Thousand to millions of distinct compounds can be synthesized through a variation of solid-phase synthesis that treats each solid-phase particle (typically a bead) as a separate reaction vessel. By splitting and pooling the collection of synthesis beads over a reaction sequence, all possible combinations of a large matrix of reagents and building blocks can be accessed, generating an enormous amplification in the number of different compounds produced using a small number of reactions (Furka, Á., et al., Int. J. Pept. Protein Res., 1991, 37, 487-493; Lam, K., et al., Nature, 1991, 354, 82-84; Houghten, R., et al., Nature, 1991, 354, 84-86). However, despite the introduction of the split-pool method almost a decade ago, its use has been limited because of challenges in compound identification, the minute quantities of released compounds, and the resulting tendency to screen compounds as mixtures (Tan, D., et al., J. Am. Chem. Soc., 1999, 121, 9073-9087). The present invention addresses these limitations, among others.

Given a library of compounds it will typically be desirable to be able to perform multiple assays with each compound. The inventors have recognized that typical synthesis beads yield insufficient quantities of compounds to allow many experiments to be performed with the amount of compound derived from a single bead. In order to overcome this limitation, the inventors have developed a novel approach to library generation in which small molecules are synthesized on large beads, capable of supporting synthesis of at least 50 nmol of compound. Following cleavage from the beads capable of supporting between 50-100 nmol of compound, the compounds yield ˜5-10 mM solutions when resuspended in 10 μl of solvent. These solutions may serve as stock solutions for the performance of numerous biological and/or chemical assays. Since each stock solution contains compound derived from a single bead, the inventors refer to this approach as the “one-bead, one-stock solution” approach. When used in miniaturized assays (e.g., with assay volumes of approximately 2-40 μl), the inventive methodology permits hundreds of assays to be performed at screening concentrations of up to 100 μM after dilution into a high density assay plate. For example, blunt-end or quill pins can be used to transfer nanoliter volumes. Representative assays include cytoblot assays (Stockwell, B., et al., Chem. Biol., 7, 275-286, 1999), protein-binding assays using small molecule microarrays (See, e.g., MacBeath, G., et al., J. Am. Chem. Soc., 121, 7967-7968, 1999; Hergenrother, P., et al., J. Am. Chem. Soc., 122, 7849-7850, 2000.)

Thus, in one aspect the invention provides a method of performing solid-phase synthesis of a target compound comprising steps of: (i) providing a solid support having a capacity to support synthesis of at least 50 nmol of compound; (ii) attaching a linker to the solid support; (iii) reacting a substrate with the linker, thereby loading the substrate onto the solid support; (iv) treating the support-bound substrate with a suitable reagent under suitable conditions to effect a desired chemical transformation; and (v) optionally repeating step (iv) until desired functionalization of the substrate is achieved, thereby forming a support-bound target compound. The term “target compound” is used for convenience only and is not intended to suggest that a practitioner of the invention must have a particular compound in mind or that the method is intended to result in synthesis of any particular compound although such a possibility is included within the scope of the invention. According to certain embodiments of the invention the solid support is capable of supporting synthesis of at least 100 nmol of compound. According to certain embodiments of the invention the the solid support is capable of supporting synthesis of at least 200 nmol of compound. In certain embodiments of the invention the solid support is capable of supporting between 50-100 nmol of compound, inclusive. Any other subranges are also included within the scope of the invention.

According to certain embodiments of the invention the solid support is a bead. According to certain embodiments of the invention the solid support is a grafted polymeric support such as a lantern. In general, any linker may be used in the practice of the inventive solid-phase synthesis and screening methods described herein, including, but not limited to, the linkers described below. The method may further include the step of (vi) activating the linker prior to reacting the substrate with the linker. The methods may further include one or more of the following steps: (vi) dispensing the solid support into a vessel; (vii) cleaving the target compound from the solid support; (vii) dissolving the cleaved compound in a suitable solvent; and (ix) screening the compound for a biological or chemical activity.

Any of the inventive methods may be performed using a plurality of solid supports, a plurality of substrates, and a plurality of suitable reagents, thereby providing methods of producing a library of target compounds. The invention also provides methods of screening a compound library comprising steps of: (i) arraying a plurality of support-bound target compounds prepared according to any of the methods described above for performing solid-phase synthesis of a target compound into a plurality of individual vessels; (ii) cleaving the target compounds from the solid supports; (iii) dissolving the cleaved target compounds in a solvent; and (iv) screening the compounds for biological or chemical activity. According to various embodiments of the invention the wells in the foregoing methods may be wells of a microtiter plate suitable for high-throughput screening, such as a 96-well plate, 384-well plate, 1596-well plate, etc.

The inventive methods may also include steps of encoding and decoding the solid supports and/or compounds as described in more detail below. For example, each of steps (iii), (iv), and (v) may be encoded. According to certain embodiments of the invention encoding is achieved by performing a reaction in which a chemical tag is inserted into the solid support, the substrate, the compound being synthesized, or any combination of these. The tag may be, for example, a chloroaromatic diazoketone tag. Thus the invention provides methods of performing solid-phase synthesis of a target compound in which the encoding step comprises labeling the solid support, the substrate, the target compound or any combination of the foregoing with one or more chemical tags prior to or subsequent to performing each of steps (iii), (iv) and (v), wherein the tags are characteristic of the substrate identity and the reaction sequence performed in steps (iv) and (v).

According to certain embodiments of the invention certain of the steps may be performed robotically. These include, in particular, the dispensing or arraying, cleaving, and resuspending steps. In addition, steps including, but not limited to, steps of transferring liquids from a stock solution into a vessel for performing a biological or chemical screen, for printing onto a surface, etc., can be performed robotically.

It will be appreciated that the above methods may be performed using a plurality of individual solid supports, substrates, and reagents using a split-pool approach, thereby generating a combinatorial library. The invention provides compound libraries, also referred to as collections of compounds, produced according to the methods described above. According to different embodiments of the invention the collection includes at least 100, at least 1000, at least 2,000, or at least 10,000 compounds. Any subranges are also included within the scope of the invention.

In addition to the inventive methods for library generation and screening, the present invention provides a number of reagents for use in performing the methods. In particular, the invention provides high capacity solid supports capable of supporting synthesis of at least 50 nmol of compound, at least 100 nmol of compound, at least 200 nmol of compound, and any subranges thereof. For example, the invention provides bead/linker combinations in which the beads are functionalized with a linker suitable for library synthesis. The invention also provides polystyrene-grafted supports including polystyrene-grafted lanterns functionalized with such linkers.

Solid Supports

Beads. As mentioned above, according to certain embodiments of the invention the solid supports comprise beads having a capacity to support synthesis of at least 50 nanomoles (nmol) of compound. According to certain embodiments of the invention the beads have the capacity to support synthesis of between approximately 50 and 100 nmol of compound. According to certain embodiments of the invention the beads have the capacity to support synthesis of between approximately 100 and 200 nmol of compound. Any subranges of the foregoing are also included within the scope of the invention. As used herein, an individual solid support (e.g., a bead) has the capacity to support synthesis of X nmol of compound if the solid support includes X nmol of a reactive functional group to which a chemical moiety that can serve as a linker may be attached. Typically the reactive functional group forms part of or is covalently bound, either directly or indirectly, to the solid support. For example, as described in Example 3 the inventors have produced polystyrene beads in which at least 50 nmol bromine (Br) is added to each bead and have attached linkers to the solid supports via a Suzuki coupling reaction. Any of a wide variety of functional groups known in the art may be incorporated into the solid support to allow attachment of a linker. As just one example, other halogen atoms such as iodine may be used.

In order that the beads may be used in a “one-bead one-stock solution” approach as described further in Examples 1 and 2, it is preferable that the dimensions of the beads are compatible with dispensing the bead into an individual vessel such as the well of a microtiter plate suitable for high throughput screening, e.g., a 96-well, 384-well, or 1596 well plate, many of which are commercially available.

The beads are typically formed from a polymeric material such as polystyrene. Other materials that can be used to form beads include polymethylacrylamide, macroporous polystyrene, polyethylene, etc. Either polymers or copolymers may be used. For example, beads comprised of polystyrene-polyethylene glycol copolymers have been developed. The polymeric material may, but need not be, be cross-linked, e.g., with divinylbenzene or another appropriate cross-linking agent. According to certain embodiments of the invention the beads have a diameter of between approximately 400 and 600 μm or between approximately 500 and 600 μm. Any subranges are also included within the scope of the invention. Such beads may be referred to herein as large beads or macrobeads. Suitable beads include, e.g., 400-450 μm and 500-560 μm 1% divinylbenzene (DVB) cross-linked polystyrene beads available from Rapp Polymere, GmbH, Emst-Simon-Str. 9, D 72072 Tuibingen, Germany and 500-600 mm 1% DBV cross-linked polystyrene beads available from Polymer Laboratories, Amherst Fields Research Park, 160 Old Farm Road, Amherst, Mass. Such beads require functionalization as an initial step for library synthesis. Pre-functionalized beads are also commercially available. For example, 410-500 and 500-600 mm 1% DBV cross-linked p-bromostyrene/styrene copolymerized resins loaded with between 1.0 and 2.0 milliequivalents of Br per gram are available from Polymer Laboratories. However, any beads having a similar diameter may be employed. Preferably the beads are physically robust and able to withstand manipulation.

It will be appreciated that the majority of compounds in a synthetic library will be unlikely to demonstrate significant biological and/or chemical activity even if screened using a large number of assays. Given that this is the case, the inventors have recognized that it may be preferable to limit the quantity of each compound synthesized to the range of less than 1 mg of compound (e.g., approximately 0.1 mg or 100-200 nmol) rather than employing an approach that yields larger quantities of compound. Limiting the amount of compound is more cost-effective and environmentally sound.

Solid Supports Having Grafted Polymeric Surfaces. As mentioned above, typically it will be desirable to synthesize the compounds in a compound library in limited quantities in order to minimize cost, disposal considerations, etc. However, once compounds of interest are identified via biological and/or chemical screens, it will often be desirable to synthesize these compounds in larger quantities (e.g., milligrams to grams) for further testing, etc. The term “resynthesis” will be used herein to refer to a situation in which a compound initially synthesized as a component of a compound library (e.g, a compound synthesized according to a split-pool synthesis approach) is then individually synthesized in larger quantities.

Since the reactions employed in resynthesis of any particular compound of interest will initially be a subset of those employed in the synthesis of the library from which it comes, many of the same considerations apply. For example, solid-phase synthetic approaches are similarly attractive. One approach to resynthesis involves simply utilizing large numbers of individual beads such as those described above. However, the inventors have recognized that utilization of a single solid support rather than multiple beads offers a number of advantages. For example, when a single support is used each support can be treated as a single reaction vessel and is more easily manipulated than large quantities of beads. Single supports can be used directly in split-pool synthesis whereas large quantities of beads require a secondary container for use in split-pool synthesis, an approach which is referred to as “tea-bagging” the resin. Of course the grafted polymeric supports, (e.g., lanterns) of the invention may be used for a variety of purposes other than resynthesis.

The inventors have recognized that a class of pellicular solid supports comprising a polymeric surface grafted onto a rigid base polymer have significant advantages as a platform for resynthesis. Typically these supports are formed from a rigid base polymer that provides mechanical strength, allowing the support to be larger than a typical bead and permitting it to be shaped in a variety of different ways. For example, the support may be shaped into a variety of three-dimensional forms including lanterns, crowns, gears, pins, pucks, discs, beads, microtitre plates or sheets. A feature of these supports is that the surface area rather than the volume determines loading capacity. Certain of these three-dimensional forms are modular in that they comprise a, plurality of physical units which are suitable for use in a set of simultaneous or sequential chemical reactions, and which provide reproducible chemical properties. This feature offers the advantage in that the user can monitor the effectiveness of every synthetic step in the synthesis of each molecule by removing and analyzing a small portion of the support, a possibility that is limited when alternative solutions such as those described in Atrash, B., et al., Agnew Chem. Int. Ed., 2001, 40, 938-941, are used. The surface of the rigid base polymer is grafted with a second polymer, e.g., a mobile polymer, to which functional groups may be attached either directly or indirectly. Exemplary grafted polymeric supports are described, for example, in WO 02/06384. See also Rasoul, F. et al., Biopolymers (Peptide Science), 2000, 55, 207-216 for further discussion. For purposes of the present invention the grafted polymeric surface need not be activated in precisely the manners described therein. Instead, the grafted polymeric surface is modified to include a reactive functional group suitable for attachment of a chemical moiety to which a substrate may be attached as an initial step in library synthesis. For example, as described further in Example 5, the inventors have functionalized the grafted polymeric surface with bromine in order to facilitate addition of a linker to the solid support.

Any of a number of polymers may be used as the rigid base polymer. For example, an optionally substituted polyolefin, silicone polymer, natural or synthetic rubber, polyurethane, polyamide, polyester, formaldehyde resin, polycarbonate, polyoxymethylene, polyether, or epoxy resion, or a co-polymer of any of the foregoing, such as a co-polymer of polyethylene and polypropylene. Optionally substituted polyolefins may be selected from polyalkenes, such as polyethylene, polypropylene, and polyisobutylene; acrulic polymers such as polyacrylate, polymethacrylate, and polyethylacrylate; vinyl halide polymers, such as polyvinylchloride, fluoropolymers, polyvinylethers, polyvinylidine halides, polyacrylonitrile, polyvinylketones, polyvinyl aromatics and polyvinyl esters. See also Hans Dominghaus, Plastics for Engineers: Materials, Properties, Applications (Hanser Publishers, New York, 1992) for further examples. For purposes of the present invention polystyrene is preferred as the base polymer.

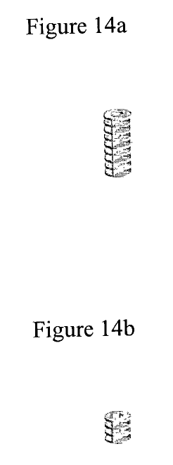

A variety of different polymers may be used as the second polymer, which is grafted onto the surface of the rigid base polymer. For example, polyvinyls, polystyrenes, poly-α-methylstyrenes, polyvinylalcohols such as polyvinyl acetate, polyacrylates, polyacrylamides, polyetheylene glycols, polylactic acids and derivatives, blends, and co-polymers of the foregoing. For purposes of the present invention polystyrene is preferred. The second polymer may be cross-linked using an appropriate cross-linking agent. For example, as described in more detail in Example 5, according to certain embodiments of the invention the second polymer is polystyrene. In particular embodiments of the invention the grafted polymeric support is a lantern such as those available from Mimotopes, Pty Ltd, Duerdin St., Clayton, Victoria 3168, Australia (See Web site having URL www.mimotopes.com), e.g., a polystyrene-grafted lantern such as a SynPhase-PS lantern. SynPhase PS D-series Lanterns™ have a nominal loading of 35 μmol (PS), while SynPhase PS L-series Lanterns™ have a nominal loading ,of 15 μmol. FIG. 14 depicts a D series (FIG. 14a) and an L series (FIG. 14b) lantern.

The invention provides grafted polymeric supports functionalized with a variety of linkers, which are described further below.

Linkers and Support/Linker Combinations

The solid supports of the invention (both large capacity beads ands grafted polymeric supports) may be derivatized with a variety of linkers. In certain embodiments of the invention the linker is an alkylsilyl linker, e.g., the linker includes a silicon atom attached to the solid support by an alkyl chain. (See Greene and Wuts, pp. 113-148, referenced above, for discussion of silyl ethers.)

According to certain embodiments of the invention the linker has the following structure:

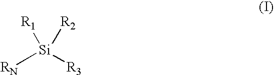

wherein R_{N}, is an aliphatic or heteroaliphatic moiety, wherein R_{N} is attached to the solid support; R₁ and R₂ are each independently hydrogen or an aliphatic, heteroaliphatic, aryl or heteroaryl moiety; and R₃ is an aliphatic, heteroaliphatic, aryl or heteroaryl moiety; halogen, —OSO₂Rₓ; wherein Rₓ is an aliphatic, heteroaliphatic, aryl or heteroaryl moiety; or O, S, —NR^{A} or —CR^{A}R^{B}; wherein any two adjacent —CR^{A}R^{B} moieties may be linked by a single or double bond as valency permits; wherein each occurrence of R_{A} and R_{B} is independently absent, hydrogen, an aliphatic, heteroaliphatic, aryl or heteroaryl moiety, halogen, —CN, —S(O)ₘRₙ, —NO₂, —COR^{J}, —CO₂R^{J}, —NR^{J}COR^{J}, —NR^{J}(CO)NR^{J}R^{J}, —NR^{J}CO₂R^{J}, —CONR^{J}R^{J}, —CO(NOR^{J})R, or —ZR^{J}, wherein Z is —O—, —S—, or NR^{K}; wherein each occurrence of R^{J} and R^{K} is independently hydrogen, —COR^{J}, —CO₂R^{J}, —CONR^{J}R^{J}, —CO(NOR^{J})R^{J}, or an aliphatic, heteroaliphatic, aryl or heteroaryl moiety, and m is 1 or 2; wherein each of the foregoing aliphatic or heteroaliphatic moieties may be substituted or unsubstituted, cyclic or acyclic, linear or branched, saturated or unsaturated; and each of the foregoing aryl and heteroaryl moieties may be substituted or unsubstituted.

In certain embodiments of the invention R₁ and R₂ are each independently alkyl, heterolalkyl, aryl or heteroaryl; wherein each of the foregoing alkyl or heteroalkyl moieties may be substituted or unsubstituted, cyclic or acyclic, linear or branched, saturated or unsaturated.

In certain embodiments of the invention R₃ is aryl, heteroaryl, alkenyl, heteroalkenyl, halogen, or —OSO₂R,; and Rₓ is an aliphatic, heteroaliphatic, aryl or heteroaryl moiety; wherein each of the foregoing alkyl, alkenyl, heteroalkenyl, moieties may be substituted or unsubstituted, cyclic or acyclic, linear or branched, saturated or unsaturated; and each of the foregoing aryl and heteroaryl moieties may be substituted or unsubstituted.

According to certain embodiments of the invention R_{N} is covalently attached to the solid support. R_{N} may be a linear hydrocarbon chain. According to certain embodiments of the invention R_{N} is an aliphatic or heteroaliphatic moiety between 1 and 20 atoms in length, between 1 and 10 atoms in length, or between 1 and 5 atoms in length. In certain embodiments of the invention R₁ and R₂ are each independently substituted or unsubstituted lower alkyl, lower heteroalkyl, aryl or heteroaryl. In certain embodiments of the invention R_{N}, R₁, and R₂ do not contain heteroatoms while in certain embodiments of the invention R_{N}, R₁, and R₂ do not contain double bonds. Wherever length ranges are used herein (e.g., between 1 and 20 atoms in length) it is to be understood that any and all subranges may be included within the scope of the invention even if not explicitly recited herein.

According to particular embodiments of the invention R₁ and R₂ are each independently methyl, ethyl, n-propyl, isopropyl, n-butyl, sec-butyl, isobutyl, tert-butyl or phenyl. For example, according to one embodiment of the invention described in further detail in the Examples, R₁ and R₂ are each isopropyl.

In certain embodiments of the invention the silicon-containing linker has the structure:

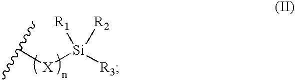

wherein R₁ and R₂ are each independently alkyl, heteroalkyl, aryl or heteroaryl; R₃ is aryl, heteroaryl, alkenyl, heteroalkenyl, halogen, or —OSO₂Rₓ; wherein Rₓ is an aliphatic, heteroaliphatic, aryl or heteroaryl moiety; X is O, S, —NR^{A} or CR^{A}R^{B}; wherein any two adjacent —CR^{A}R^{B} moieties may be linked by a single or double bond as valency permits; wherein each occurrence of R_{A} and R_{B} is independently absent, hydrogen, an aliphatic, heteroaliphatic, aryl or heteroaryl moiety, halogen, —CN, —S(O)ₘR^{J}, —NO₂, —COR^{J}, —CO₂R^{J}, —NR^{J}COR^{J}, —NR(CO)NR^{J}R^{J}, —NR^{J}CO₂R^{J}, —CONR^{J}R^{J}, —CO(NOR^{J})R^{J}, or —ZR^{J}, wherein Z is —O—, —S—, or NR^{K}; wherein each occurrence of R^{J} and R^{K} is independently hydrogen, —COR^{J}, —CO₂R^{J}, —CONR^{J}R^{J}, —CO(NOR^{J})R^{J}, or an aliphatic, heteroaliphatic, aryl or heteroaryl moiety, and m is 1 or 2; and n is an integer from 1-10; wherein each of the foregoing alkyl, alkenyl, heteroalkenyl, heteroalkyl, aliphatic and heteroaliphatic moieties may be substituted or unsubstituted, cyclic or acyclic, linear or branched; and each of the foregoing aryl and heteroaryl moieties may be substituted or unsubstituted.

According to certain embodiments of the invention X is —CR^{A}R^{B}; wherein any two adjacent —CR^{A}R^{B} moieties may be linked by a single or double bond as valency permits; wherein each occurrence of R_{A} and R_{B} is independently absent, hydrogen, an aliphatic, heteroaliphatic, aryl or heteroaryl moiety, halogen, —CN, —S(O)ₘR^{J}, —NO₂, —COR^{J}, —CO₂R^{J}, —NR^{J}COR^{J}, —NR^{J}(CO)NR^{J}R^{J}, —NR^{J}CO₂R^{J}, —CONR^{J}R^{J}, —CO(NOR^{J})R^{J}, —ZR^{J}, wherein Z is —O—, —S—, or NR^{K}; wherein each occurrence of R^{J} and R^{K} is independently hydrogen, —COR^{J}, —CO₂R^{J}, —CONR^{J}R^{J}, —CO(NOR^{J})R^{J}, or an aliphatic, heteroaliphatic, aryl or heteroaryl moiety, and m is 1 or 2; and n is an integer from 1-5; wherein each of the foregoing aliphatic and heteroaliphatic moieties may be substituted or unsubstituted, cyclic or acyclic, linear or branched; and each of the foregoing aryl and heteroaryl moieties may be substituted or unsubstituted.

In certain embodiments of the invention X is —CR^{A}R^{B}; wherein each occurrence of R_{A} and R_{B} is independently hydrogen, lower alkyl, lower heteroalkyl, aryl, heteroaryl, halogen, —CN, —S(O)ₘR^{J}, —NO₂, —COR^{J}, —CO₂R^{J}, —NR^{J}COR^{J}, —NR^{J}(CO)NR^{J}R^{J}, —NR^{J}CO₂R^{J}, —CONR^{J}R^{J}, —CO(NOR^{J})R^{J}, or —ZR^{J}, wherein Z is —O—, —S—, or NR^{K}; wherein each occurrence of R^{J} and R^{K} is independently hydrogen, —COR^{J}, —CO₂R^{J}, —CONR^{J}R^{J}, —CO(NOR^{J})R^{J}, or an aliphatic, heteroaliphatic, aryl or heteroaryl moiety, and m is 1 or 2; and n is an integer from 1-5; wherein each of the foregoing alkyl, heteroalkyl, aliphatic and heteroaliphatic moieties may be substituted or unsubstituted, cyclic or acyclic, linear or branched; and each of the foregoing aryl and heteroaryl moieties may be substituted or unsubstituted.

According to certain embodiments of the invention each occurrence of X is —CH₂; and n is an integer from 1-5. According to certain embodiments of the invention each occurrence of X is —CH₂; and n is 3. In certain embodiments of the invention R₁ and R₂ are each independently substituted or unsubstituted lower alkyl, lower heteroalkyl, aryl or heteroaryl. In various embodiments of the invention R₁ and R₂ are each independently methyl, ethyl, n-propyl, isopropyl, n-butyl, sec-butyl, isobutyl, tert-butyl or phenyl. In a particular embodiment of the invention R₁ and R₂ are each isopropyl. According to certain embodiments of the invention R₃ is aryl, heteroaryl, alkenyl, heteroalkenyl, halogen, or —OSO₂CF₃; wherein each of the foregoing alkenyl and heteroalkenyl moieties may be substituted or unsubstituted, cyclic or acyclic, linear or branched; and each of the foregoing aryl and heteroaryl moieties may be substituted or unsubstituted. According to particular embodiments of the invention R₃ is substituted aryl, substituted phenyl, or phenyl substituted with one or more occurrences of halogen, lower alkyl or lower alkoxy.

According to certain embodiments of the invention R₃ is a moiety having the structure:

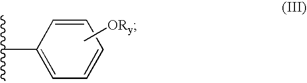

wherein R_{y} is lower alkyl.

According to a particular embodiment of the invention the silicon-containing linker has the structure:

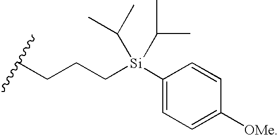

Example 3 describes the synthesis of the linker depicted above and its attachment to a large PS bead in detail. However, it will be appreciated that the reactions described in Example 3 may readily be adapted to synthesis of a wide variety of similar linkers. As described in Example 3, a silicon-functionalized alkyl borane may be synthesized using commercially available starting materials (starting from diisopropylchlorosilane in the case of a diisopropylalkylsilane linker). The silicon-functionalized alkyl borane may be used as the substrate in a Suzuki coupling reaction with bromine-functionalized beads. The linker may be activated as a trialkylsilyl triflate by, for example, treatment with trifluoromethanesulfonic acid (triflic acid) followed by removal of excess acid, e.g., by washing with CH₂Cl₂. In a particular embodiment the invention provides an activated solid support comprising a silicon-containing linker, wherein the silicon-containing linker has the structure:

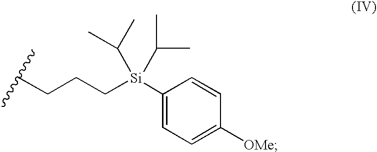

and the solid support is activated by a method comprising treating the functionalized solid support with triflic acid, thereby generating a solid support functionalized with a silicon-containing linker having the structure:

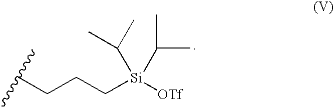

Other means of activating the linker are known to those of ordinary skill in the art and may also be used. As just one example, treatment with anhydrous hydrogen chloride in organic solvents such as CH₂Cl₂ can be employed.

Small molecules (substrates) can be loaded onto the linker using any appropriate reaction conditions suitable for the molecules to be loaded. For example, to load alcohols following activation of the linker, a reaction sequence involving addition of 2,6-lutidine followed by addition of the alcohol may be used. Appropriate means of loading substrates with different characteristics are known in the art.

A significant feature of the silicon linkers of the invention is the ease with which they undergo Si—O bond cleavage in the presence of HF/pyridine in tetrahydrofuran (THF). This reagent is dispensable by an automated liquid handler, making it particularly useful for large numbers of 384-well microplates in which synthesis beads may be spatially arrayed in a one bead-one well format. Excess HF can be quenched, e.g., with methoxytrimethylsilane or ethoxytrimethylsilane, which yields the released small molecule and volatile by-products, the latter of which can be removed under vacuum. After drying, the resulting compound can be eluted from the bead by repetitive washing, e.g., with acetonitrile or dimethylformamide. This procedure generally results in compounds of a high purity. Accordingly, this method of cleavage is preferred in certain embodiments of the invention although other methods may also be employed such as the use of standard sources of fluoride ion known in the art.

Coding and Decoding Methodology

To allow compound identification following split-pool syntheses, a variety of encoding and decoding strategies have been developed that allow the identity of the compounds to be inferred post-synthesis (See, e.g., Czarnik, A., Curr. Opin. Chem. Biol., 1997, 1, 60-66 and references therein). in general, any suitable encoding/decoding strategy may be used in conjunction with the solid support/linker systems of the invention. In most encoding schemes the chemical reaction and building block (BB) used to synthesize each compound are encoded. Thus the synthetic history of the compound is encoded rather than its direct chemical identity. Encoding techniques include spatial, chemical, spectrometeric, electronic, and physical methods. While in general any encoding/decoding technique may be employed in conjunction with the solid support/linker systems of the invention, according to certain preferred embodiments of the inventive methods, a chemical encoding strategy similar to that described by Still is employed (Ohlmeyer, M. H. J., et al., Proc. Natl. Acad. Sci. USA, 1993, 90, 10922-10926. The method uses chloroaromatic, diazoketone tags that may be introduced via a Rh₂(O₂CC(Ph)₃)₄ catalyzed acylcarbene insertion into the aromatic rings of a PS solid support. The chloroaromatic portion of the tags may be cleaved from the support, e.g., with ceric ammonium nitrate to yield free alcohols that can then be detected using, for example, electron capture gas chromatography (GC/ECD). Each tag encodes a synthetic step (reactant and order of the step in the synthetic pathway). Thus the identity of the tags associated with a particular bead reveals the synthetic pathway to which the bead was subjected.

The inventors have modified the above tagging approach so that it can be used in conjunction with large PS beads rather than the smaller resins that have typically been used. It will be appreciated that large PS beads have markedly different physical properties from those of previously used resins. Therefore, previously reported encoding and decoding procedures were not transferable simply by scaling the procedures relative to bead size. Rather, significant modification and optimization was necessary in order to encode synthetic pathways effectively using chemical tags. A more detailed description of the modification and optimization protocol is provided in Example 4.

Accordingly, the invention provides a method of preparing a library comprising steps of: (i) providing a solid support having a capacity to support synthesis of at least 50 nmol of compound; (ii) attaching a linker to the solid support; (iii) reacting a substrate with the linker, thereby loading the substrate onto the solid support; (iv) treating the support-bound substrate with a suitable reagent under suitable conditions to effect a desired chemical transformation; and (v) optionally repeating step (iv) until desired functionalization of the substrate is achieved, thereby forming a support-bound target compound wherein each of steps (iii), (iv), and (vi) may be encoded. According to certain embodiments of the invention encoding is achieved by performing a reaction in which a chemical tag is inserted into the solid support, the substrate, the compound being synthesized, or any combination of these. The tag may be, for example, a chloroaromatic diazoketone tag. Isotope encoded tags (for decoding by mass spectrometry analysis) are also known.

Standard bead decoding is typically performed following cleavage of the compound from the bead. However, the inventors have discovered that while the integrity of the synthesized compound attached to the macrobead is not adversely affected by the chemical encoding process, sufficient tag is inserted into the compound itself to identify the tags (and thereby decode the compound) reliably by subjecting a fraction of the compound itself following cleavage from the bead (e.g., a fraction of solid compound obtained following cleavage from the bead 1-5% of a stock solution in which the compound is dissolved) to analytical procedures (e.g., GC/ECD analysis) using conditions such as those suitable for bead decoding. A representative optimized decoding protocol is described in detail in Example 4. As described in that Example, the signals obtained when the compound itself was decoded were significantly stronger (˜100 times) than those detected from bead decoding performed after compound cleavage. While not wishing to be bound by any theory, the inventors suggest that the reason for the apparent increase in tag amount relative to standard bead decoding may be due to higher efficiency of the homogeneous oxidative cleavage reaction in solution, relative to the heterogenous reaction on the support.

This discovery affords a new approach to the decoding of chemically encoded libraries. Decoding the compound itself offers a number of significant advantages relative to decoding the bead. For example, stock solution decoding can be faster, minimizes storage requirements since beads can be discarded, and is amenable to automation using a liquid-handling robot. Results obtained using four different diversity-oriented synthetic pathways indicate that stock solution decoding is feasible with many different chemistries.

Accordingly, the invention provides a method for encoding and decoding the identity of one or more members of a compound library comprising (i) performing sequences of chemical reactions resulting in synthesized compounds, wherein each chemical reaction is encoded by incorporation of a chemical tag into the compound being synthesized; and (ii) determining the reaction sequence of a synthesized compound by identifying the tags inserted into the compound. According to certain embodiments of the invention the sequence of chemical reactions is performed as a solid-phase synthesis. As described above, the method may include the step of cleaving the compound from the solid support prior to identification of the tags, resuspending the compound in a solvent, and identifying the tags by analyzing a portion of the resulting solution. The tags may be chloroaromatic diazoketone tags, for example, but other tags may also be used.

Equivalents

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The following examples contain important additional information, exemplification and guidance which can be adapted to the practice of this invention in its various embodiments and the equivalents thereof.

### EXEMPLIFICATION

### Example 1

Development of a High-Throughput Synthesis and Screening Platform: Synthesis of a High Capacity Solid-Phase Bead/Linker System, Development of a Library Encoding Strategy, and Design of Compound Decoding Methods.

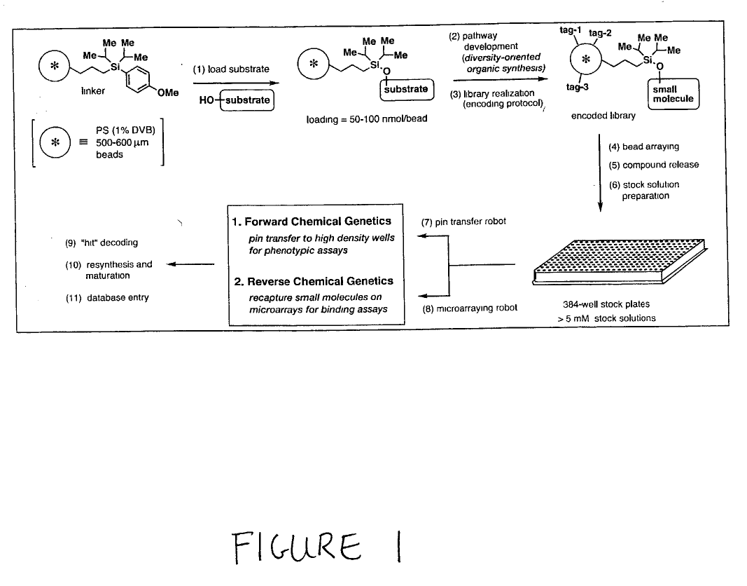

Introduction. An exemplary embodiment of the system that we have developed for high throughput synthesis and screening is outlined in FIG. 1. Small molecules are synthesized on polystyrene (PS) macrobeads, which serve as individual reaction vessels during split-pool library syntheses, delivering arrayed ˜5 mM stock solutions upon compound cleavage and resuspension in high-density assay plates [14]. This example describe the first phase of the development of a two-part “one-bead, one-stock solution” technology platform, including a scaled synthesis of a high-capacity solid-phase bead/linker system and the development of a reliable library encoding/decoding strategy. This phase was validated by the analysis of an enantioselective, diversity-oriented synthesis resulting in an encoded 4320-member library of structurally complex dihydropyrancarboxamides (12) whose synthesis has been reported [15]. A full account of the second phase incorporating: (1) bead arraying, (2) automated compound cleavage, elution, and resuspension as segregated stock solutions, and (3) assay format design and annotation is provided in Example 2.

We had previously attached compounds to 90 μm TentaGel beads [18], a poly(ethyleneglycol)-PS copolymer, via a photolabile linker [19]. This bead/linker combination allowed compounds to be released from the polymeric supports in the presence of aqueous media and living cells [20] by exposure to long wavelength (365 nm) ultraviolet (UV) light. Each split-pool synthesis step was encoded with electrophoric tags using the method introduced by Still and co-workers (see below) [21,22]. However, we observed that the manual arraying of TentaGel beads in a one bead per well format resulted in a variable distribution of beads in the assay plate wells (i.e. from 0 to 10 beads per well). After compound cleavage, numerous wells appeared to contain active compounds in cytoblot [23] assays. However, upon closer inspection, most of these ‘active’ wells held more than one synthesis bead, and thus contained a mixture of compounds. Some of the apparent activities were ephemeral, presumably a non-specific effect resulting from high concentrations of the compound mixtures. As we wished to decode and re-synthesize each compound in order to confirm a positive in an assay, or to test lower concentrations in order to determine the potency of a compound, we wished to improve upon this approach.

We sought to develop a library realization platform that produces sufficient compound per bead to perform many hundreds of assays; i.e. a minimum of approximately 50 nmol of small molecule per synthesis bead. To this end, we have developed 500-600 μm PS macrobeads for that yield ˜100 nmol of synthetic compound per macrobead [24]. Due to the substantially larger size of the PS macrobeads, they can be arrayed reliably and efficiently (e.g., ˜5 min per plate) into individual wells of standard 384-well assay plates [14,16].

Results and Discussion

Development of a Robust Bead/Linker System

We desired a bead/linker combination amenable to a wide array of reactions used inmodem organic synthesis [24]. Beyond chemical stability requirements, two other criteria were addressed regarding our bead/linker: (1) library starting materials (building blocks (BBs)) should be loaded onto the support with high efficiency, and (2) quantitative release of the final small molecule products from the support. We chose a silicon-based linker as the solution [26,27]. An important parameter of the bead/linker equation was the size and nature of the polymer support; 500-600 μm, lightly cross-linked (1% divinylbenzene), PS macrobeads are commercially available and have the physical capacity to deliver ˜50 nmol/bead. Moreover, it was the most physically robust support in our hands (see below), in comparison to either high-capacity ArgoPorel (Argonaut Technologies) or TentaGel [18] resins. The synthesis of the bead/linker system, activation of the linker, and an exemplary library synthesis are described in further detail in Example 3.

Maintenance of bead integrity throughout solid-phase synthesis. While the use of sequences of tandem organic reactions can efficiently generate complex molecules in diversity-oriented syntheses [8], we have observed that successive organic transformations, coupled with rigorous bead washing between reactions, can damage the PS macrobeads.

We wished to isolate one physically intact,bead per well prior to compound cleavage for several reasons. First, fragments of beads yield weaker compound stock solutions after bead dispensing or arraying, cleavage, and resuspension. Second, the possibility of isolating more than one fragment per well allows for stock solution contamination and the concomitant incorrect decoding of that well. To reduce these problems, we have developed a set of standard practices for bead handling during library synthesis and encoding that dramatically minimize the possibility of bead breakage. In general, we have found that the less we handle the solid supports physically, either by submission to chemical reactions, washing, or drying, the less bead breakage we observe. This reinforces the importance of an effective planning algorithm for diversity-oriented syntheses. Short reaction sequences yielding complex and diverse compounds not only ensure that positives can be re-synthesized readily, but also promote the integrity of the beads.

In order to quantify bead integrity, we used population size distribution measurements (obtained by light obscuration) to monitor the shift of the average particle size in a sample of beads. We first observed that the PS macrobeads were fragile when swollen in organic solvents. Since the use of solvents and drying are required in library synthesis, we assessed several solvent, drying, and agitation conditions. Even though certain chemical transformations appear to cause more bead breakage than others, we did not include different chemical reactions as experimental variables in our studies because we did not want to limit the types of chemistry utilized in library synthesis.

As evidence that even the most simple and gentle handling can induce damage, supports swollen in dichloromethane (CH₂Cl₂) and drained seven times, followed by overnight air drying resulted in a shift to a smaller average size distribution. As an example of extreme damage, beads were subjected to swelling in tetrahydrofuran (THF) (45 min), followed by treatment with methanol (MeOH) (45 min) and 360° rotation. The beads were then rapidly dried via lyophilization, and the whole process was repeated seven times. These supports show even more extensive damage and a greater degree of bead fragmentation. The ‘best practices’ we extrapolated from these experiments include light agitation from a wrist-action shaker, followed by blowing N₂ over the resin (30 min), and final drying under high vacuum conditions from any organic solvent. While a shift in average size still exists, these-conditions minimize fragmentation and are suitable for library syntheses, as judged by our ability to array one intact bead per well after library synthesis (see below) [14,16].

An Optimized Binary Encoding Protocol for 500-600 μm PS Macrobeads

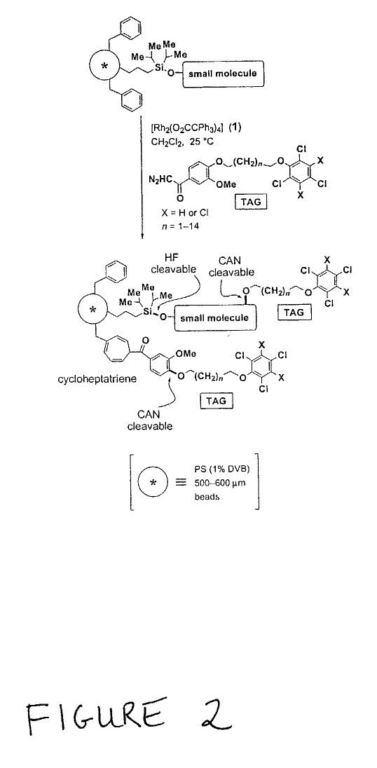

We chose to use the chemical encoding strategy [21]. This strategy is amenable to the synthesis of very large libraries (i.e. on the order of millions), operationally straightforward, and relatively inexpensive. This method uses chloroaromatic, diazoketone tags that are detectable at sub-picomolar levels by electron capture gas chromatography (GC/ECD) [22]. The tags are introduced via a Rh₂(O₂CC(Ph)₃)₄-catalyzed acylcarbene insertion into the aromatic rings of the PS solid support to yield cycloheptatrienes) [37,38]. The general reaction scheme is depicted in FIG. 2. The carbene can insert indiscriminately into both the PS support and the compound bound to the support. However, it has been postulated that the carbene inserts predominantly into the support due to the greater proportion of the support relative to compound [22]. Furthermore, the acylcarbene insertion reaction is in direct competition with the more favored carbene homodimerization reaction; therefore, the supports' tagging level is quite low relative to the bound compound (on average 1%). This inefficient tagging reaction is compensated by the exquisite sensitivity of the GC/ECD for haloaromatic functionality. The chloroaromatic portion of the tags is oxidatively labile and can be readily cleaved from the support with ceric ammonium nitrate (CAN) to yield free alcohols [39]. The tag cleavage protocol is orthogonal to our HF/py compound cleavage strategy and to the majority of our library synthesis steps (i.e. oxidative reaction conditions are preferentially avoided). After silylation with N,O-bis-(trimethylsilyl)acetimide (BSA), an aliquot of the tag ethers can be injected directly onto a GC/ECD for analysis. Each tag silyl ether has a characteristic GC/ECD retention time.

None of the libraries previously encoded using this method was synthesized on polymeric supports analogous to the high-capacity PS macrobeads we have selected. To date, the majority of the libraries encoded with chloroaromatictags [21] have been prepared on 80-100 μm TentaGel resin [18] which has markedly different physical properties from those of the large, hydrophobic PS support [40]. Preliminary experiments on 500-600 μm PS macrobeads quickly revealed that previously reported encoding and decoding procedures for 80-100 μm TentaGel were not transferable simply by scaling the procedures relative to bead size [17,41]. While not wishing to be bound by any theory, it is possible that these decoding protocols were not applicable to our hydrophobic PS support because these procedures were carried out in predominantly aqueous CAN solutions, amenable to hydrophilic TentaGel beads. Therefore, we had to develop an optimal binary encoding and decoding strategy for 500-600 μm PS macrobeads to address these issues. Moreover, we chose to develop protocols that were straightforward, lending themselves to automation in the future.

Optimization of the Encoding Procedure

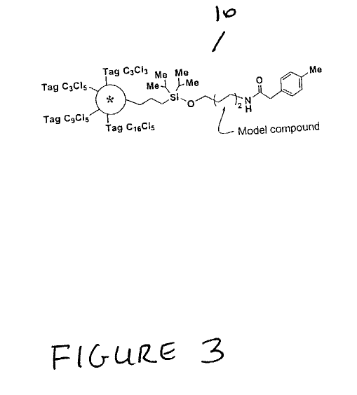

In order to mimic conditions during actual library encoding steps, we used an encoding test support on 500-600 μm PS macrobeads to which a ‘dummy compound’ was bound. N-(5-Hydroxy-pentyl)-4-methyl-benzamide [42] was chosen as the—‘dummy compound’ because it: (1) could be loaded onto the support easily through the primary alcohol moiety, (2) had low volatility, thereby expediting cleavage and analyses of small compound samples, and (3) had structural elements that would allow us to study if the carbene was indiscriminately inserting into the compound [43] versus the polymer support (see below). The support loaded with dummy compound, collectively referred to as test support 10, is depicted in FIG. 3. Finally, we decided to encode test support 10 with four tags in each encoding optimization experiment (FIG. 3): Tag C₃Cl₃ (X=H, n=1), Tag C₃Cl₅ (X=Cl, n=1), Tag C₉Cl₅ (X=Cl, n=7), and Tag C₁₆Cl₅ (X=Cl, n=14). These four tags were selected because they had GC retention times that spanned the full window of the ˜12 min GC chromatogram.

In general, an encoding step requires the addition of solutions of both tag and rhodium catalyst to the polymer support. As the PS macrobeads swell very well in CH₂Cl₂, a property necessary for good reaction kinetics on solid phase, we selected CH₂Cl₂ as the solvent for the encoding reactions. For each reaction condition we examined, 10 beads were subjected to the reaction condition and decoded in parallel (we averaged data over 10 beads due to the relatively wide size distribution of the PS macrobeads (500-600 μm)), and the resulting picomolar data for each tag on the 10 beads were averaged. While we were concerned initially with bead breakage during the tagging reactions (see above), we found that gentle tumbling during the reaction provided maximum tag incorporation; in the end, our encoding procedure was essentially non-destructive to the beads. Next, we systematically increased the tag and rhodium catalyst concentrations from concentrations previously used on TentaGel resin [17] (2 nmol tag/bead, 40 pmol catalyst/bead) to concentrations approximately 10- and 50-fold higher for tag and catalyst, respectively (20 nmol tag/bead, 2 nmol catalyst/bead). Altering the concentration of either the tag or catalyst by three orders of magnitude about these conditions demonstrated that our initial increased tag and catalyst concentrations were superior.

Results obtained by varying the experimental conditions are reported in Tables 1 and 2 in Blackwell, H., et al., Chemistry & Biology, 2001, 8, 1167-1182, and references herein to Tables 1 and 2 refer to those tables. The next experimental condition we perturbed was the order of addition of tags and rhodium catalyst (Table 1, entry 1a-e). The test support was pre-soaked in a solution of tag or catalyst for varying amounts of time, after which catalyst or tag, respectively, was added to the encoding reaction. Analysis of the relative GC peak area for each tag (post-bead decoding) indicated that pre-soaking the support in tag solution for 45 min prior to the addition of the rhodium catalyst 5 solution was optimal. We next turned to the optimization of the overall encoding reaction time (Table 1, entry 2a-c). A time course experiment was run over 24 h, before which each support sample was pre-soaked in tag solution for 45 min. Reactions were quenched at specific times throughout the time course by addition of heptylamine to the reaction mixture, which deactivated the rhodium catalyst [21]. The amount of tag covalently bound to the beads increased steadily over the course of the first 16 h, but after this point, there was no significant further tag incorporation. From this point forward, we conducted all of our encoding reactions for 16 h, after a 45 min pre-soak period with tag solution.

Compound Integrity After the Encoding Procedure

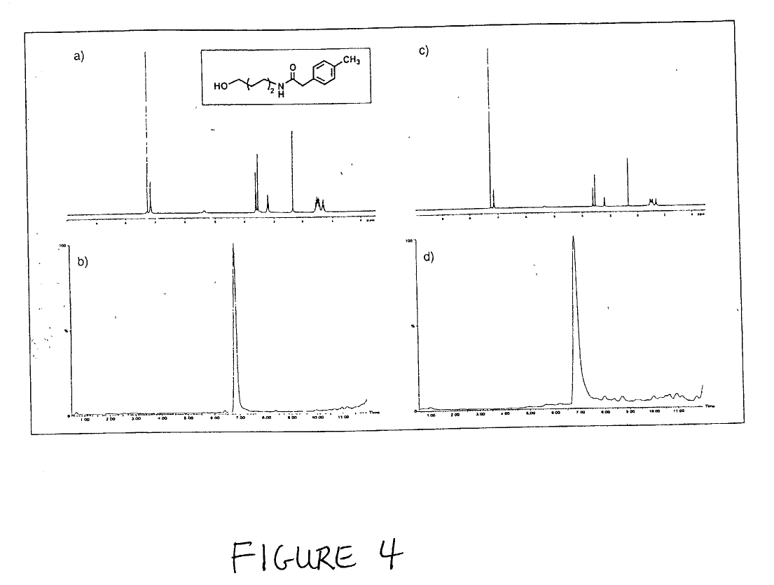

The selectivity of the carbene insertion reaction for the ‘dummy compound’ on test support and the polymeric support itself warrants discussion. We observed that the tag peaks in the GC traces for macrobeads decoded before compound cleavage were dramatically stronger than those for macrobeads decoded after compound cleavage (Table 1, entry 3a-c). Since compound cleavage will precede bead decoding routinely in our technology process, compound was cleaved from the beads prior to decoding in all of our optimization studies thereafter. To examine the degree of tag insertion into a compound relative to the PS macrobeads, we carried out experiments with the ‘dummy compound’. A portion of the test support was treated with HF/py to effect cleavage of the ‘dummy compound’ and to generate an unadulterated compound sample, before any encoding took place. The remainder of the support sample was encoded using our optimized protocol, and then treated with HF/py to release the compound. The two samples were characterized by ¹H nuclear magnetic resonance (NMR) and tandem liquid chromatography/mass spectroscopy (LC/MS), and both techniques failed to show any detectable tag incorporation into the compound (FIG. 4). Further studies have shown that the stock solutions derived from single macrobeads encoded with the described encoding protocol remain analytically pure (>99.9%).

Optimization of the Decoding Procedure

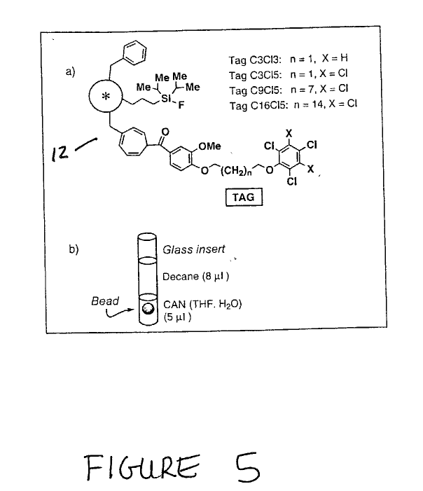

With an optimized encoding procedure in hand, we turned to the optimization of the bead decoding protocol. Support 10 was encoded using the protocol above to generate a decoding test support, from which the “dummy compound” was cleaved. FIG. 5a shows the structure of decoding test support 12 encoded with four tags. FIG. 5b shows a schematic of the glass autosampler insert used as a tag cleavage vessel. The biphasic tag cleavage cocktail consists of a lower aqueous CAN solution and an upper decane layer. Our initial decoding protocol involved multiple steps: (1) placing individual beads in glass autosampler inserts, (2) adding 5 Wl of a 0.25 M CAN solution in 1:1 THF/H2O, (3) layering onto this 8 Wl of anhydrous decane, (4) allowing the reaction to proceed for 2 h at room temperature, (5) sonicating the samples for 1 min, and finally, (6) manually removing the decane layer from the insert to a fresh glass autosampler insert, followed by silylation with BSA and injection on the GC/ECD (FIG. 5b). The PS macrobeads float near the interface of the CAN solution and decane layers. The CAN cleaves the hydrophobic chloroaromatic tags from the macrobeads, which then partitioninto the decane layer. Optimization of this multifaceted protocol required the careful scrutiny of each step. We chose not to alter the volumes of decane (8 μl) and CAN solution (5 μl) initially because these volumes are easy to manipulate either manually or in an automated format using a liquid-handling robot. First, we observed that longer decoding reaction times allowed fornmore complete tag cleavage from the beads, and we selected 21 h as the optimal reaction time (Table 2, entry 1a-d). Second, we observed that increasing the reaction temperature significantly improved the efficiency of tag cleavage (Table 2, entry 2a-c). While more tag was released at 60° C. relative to lower temperatures, we decided that 37° C. was a judicious selection because of the decreased probability of losing solvent by evaporation, a concern when working with microliter volumes. Furthermore, placing large numbers of sealed samples in a 37° C. laboratory incubator was operationally straightforward.

We next examined the effect of CAN solution concentration and solvent composition on the efficiency of the decoding procedure (Table 2, entry 3a-e). Since H₂O is required as a cosolvent in the CAN cleavage mechanism [39], we used other polar organic solvents in place of THF to investigate the optimal aqueous solvent mixture for tag cleavage. Notably, THF proved to be the best co-solvent; in fact, as we increased the ratio of THF:H2O, we saw a dramatic increase in tag cleavage. While not wishing to be bound by any theory, we attributed this to the markedly improved swelling of copolymer beads in THF relative to H2O, and therefore, better reaction kinetics within the polymer matrix. At ratios of THF:H2O higher than 5:1, CAN began to precipitate out of 0.24 M solutions. Lower CAN concentrations were investigated at higher THF:H2O solvent ratios, but no significant improvements were observed, and precipitation of CAN remained a problem. This suggested that the cleavage reaction was dependent on bead swelling and total CAN concentration. Ultimately, we selected the 5:1 THF:H2O, 0.24 M CAN solution as the optimal solution for tag cleavage. Other apolar solvent layers were not investigated because the hydrophobicity and low volatility of decane were optimal for sequestering the chloroaromatic alcohols and for sample handling, respectively.

We investigated whether a brief sonication period was necessary at the end of the decoding reaction (Table 2, entry 4a-d). However, we observed a reduction in the amount of tag observed if we omitted the sonication step completely. Sonication times up to 30 min were studied, and a 1˜10 min sonication period was chosen as the optimal post-decoding reaction procedure. Finally, we observed that silylating the tag alcohol solutions from each bead with 1 μl of a 1:1 BSA:decane solution gave the most reliable and strongest GC traces (Table 2, entry 5a-e). This hydrocarbon solution of BSA does not hydrolyze readily in open air and could be amenable to a liquid-handling robot in an automated decoding process. Of note, in a direct comparison with a recently published decoding protocol [41], our final, optimized bead decoding procedure delivered consistently larger values of cleaved tag (0.02 vs. 2 pmol of each tag) per PS macrobead, thus confirming the value of these optimization studies for 500-600 μm PS macrobeads.

Decoding Directly From Compound Stock Solutions

While the integrity of a compound attached to a macrobead is not adversely ajected by the chemical encoding process (see above), we have found that sufficient tag is inserted into the small molecule itself to decode the compound reliably by subjecting a fraction (1-5%) of its corresponding stock solution to our optimized decoding protocol [43]. Stock solution decoding is described in detail in Example 4.

Process Validation: Encoded, Enantioselective Diversity-Oriented Synthesis and Partial Decoding of a 4320-Member Library Prepared on 500-600 μm PS Macrobeads

In order to test both the PS macrobeads and the encoding/decoding protocol detailed above in an actual library synthesis, an encoded, split-pool library of 4320 dihydropyrancarboxamides (12) was synthesized featuring an R-or S-bis(oxazoline)copper (II) tri£ate-catalyzed heterocycloaddition reaction [44,45] as a key diversity-generating step. A detailed description of the pathway development phase of this research is presented in [15].

The successful synthesis and partial decoding of this library validate not only our binary encoding/decoding protocol, but also the entire synthesis platform as a reliable procedure for the generation of encoded split-pool libraries. The use of stock solution decoding further enables this platform as it simplifies the elucidation of structures of ‘hits’ from assays and lends itself to future automation. Four libraries currently underway in our laboratory are being encoded using this protocol, and preliminary data suggest that our encoding protocol is tolerant of the diverse chemical transformations contained in these four pathways.

This example lays the foundation for Example 2, in which members of libraries are distributed on a per bead basis into multiwell assay plates, submitted to automated cleavage, and resuspended to generate plates of pure, arrayed stock solutions, as described in Example 2. The individual stock solutions originating from single macrobeads have been found to be su ¤cient for hundreds of phenotypic assays (forward chemical genetics) and thousands of protein-binding assays (reverse chemical genetics) before a need for re-synthesis.

Materials and Methods

General Synthetic Methods

General reagents were obtained from Aldrich Chemical Co., Acros, or J. T. Baker and used without further purification. Reaction solvents (THF, diethyl ether, DMF, toluene, and CH₂Cl₂) were obtained from J. T. Baker (high-performance liquid chromatography (HPLC) grade) and purined by passage through two solvent columns prior to use [47]. Diusopropylethylamine and 2,6-lutidene were distilled from calcium hydride; MeOH was distilled from magnesium methoxide. Brominated PS beads (bead diameter=500-600 μm; two mequiv/g) were obtained from Polymer Labs, Inc. and derivatized with the silyl ether linker according to the procedure described in Example 3. Diazoketone chloroaromatic tags and Rh₂(O2CC(Ph)₃)₄ catalyst were purchased from Pharmacopeia, Inc. and TCI, respectively, and used without further purification. BSA was purchased in 1 ml sealed glass ampoules from Pierce Chemical Co. and used immediately after opening.

Solid-Phase Reactions Small-scale solid-phase reactions (5-10 mg resin) were performed in 500 μl polypropylene Eppendorf tubes with mixing provided by a Vortex Genie-2 vortexer fitted with a 60 microtube insert. Medium-scale solid-phase reactions (20-500 mg resin) were performed in 2 ml fritted polypropylene Bio-Spin 0 chromatography columns (Bio-Rad) or 10 ml fritted polypropylene PD-10 columns (Pharmacia Biotech) with 360° rotation on a Barnstead-Thermolyne Labquakel shaker. Large-scale solid-phase reactions (>500 mg resin) were performed in silanized 50 or 100 ml fritted glass tubes equipped for vacuum filtration and N2 bubbling. The tubes were silanized by treatment with 20% dichlorodimethylsilane/CH₂Cl₂ for 15 min, MeOH for 15 min, followed by oven heating at 120° C. for at least 2 h. After small-scale reactions, resin samples were transferred to 2 ml Bio-Spin 0 columns. Resin samples in polypropylene columns were washed on a Vac-Man 0 Laboratory Vacuum Manifold (Promega) fitted with nylon three-way stopcocks (Bio-Rad). Resin samples in glass tubes were washed in the tubes with alternating periods of N₂ bubbling and vacuum draining. The following standard wash procedure was used: 3×THF, 3×DMF, 3×THF, 3×CH2Cl2. For compound cleavage, resin samples were transferred via spatula to 500 μl Eppendorf tubes and suspended in Ar-degassed HPLC grade THF, followed by pyridine and HF/py (Aldrich, HF (70%)/py (30%)) in a ratio of 90:5:5 (e.g. 150 μl total volume for 10 mg of macrobeads). Samples were then sealed with Parafilm and gently agitated on a vortexer for 90 min. TMSOMe was added (e.g. ˜150 μl for 10 mg of macrobeads), and the samples were sealed with Parafilm and placed on a vortexer for an additional 30 min. The supernatant fluid was removed, transferred to another Eppendorf tube, and concentrated in vacuo.

Purification and Analysis

HPLC was performed on a Nest Group Hypersil C18 100 Å3 μM, 4.6 mm×6 cm column using a flow rate of 3 ml/min and a 4 min gradient of 0-99.9% CH₃CN in H2O/0.1% trifluoroacetic acid, constant 0.1% MeOH with diode array UV detection. NMR spectra were recorded on Varian Inova 500 MHz and 400 MHz instruments. Mass spectra were obtained on Jeol AX-505H or SX-102A mass spectrometers by electron impact ionization, chemical ionization with ammonia, or fast atom bombardment ionization with glycerol or 3-nitrobenzyl alcohol/sodium iodide matrices. LC/MS data were obtained on a Micromass Platform LCZ mass spectrometer in APCI mode attached to a Waters 2690 HPLC system. LC/MS chromatography was performed on a Waters Symmetry C18 3.5 WM, 2.1 mm×50 mm column using a flow rate of 0.4 ml/min and a 10 min gradient of 15-100% CH₃CN in H2O, constant 0.1% formic acid with 200-450 nmol detection on a Waters 996 photodiode array detector. GC/ECD data were obtained on a Hewlett Packard 6890 gas chromatograph fitted with a 7683 series injector and autosampler, split-splitless inlet, μ-ECD detector, and a J&W DB1 15 m×0.25 mm×0.25 Wm column. (Gradient start temperature: 110° C.; hold 1 min, ramp 45° C./min to 250³C., hold 2 min, ramp 15° C./min to 325° C., hold 2 min. Flow rate: constant flow, 1 ml/min. Inlet was purged at 1 min with flow rate 60 ml/min, reduced to 20 ml/min at 2 min). Synthesis of the ‘dummy compound’, (N-(5-hydroxy-pentyl)-4-methyl-benzamide) N-(5-Hydroxy-pentyl)-4-methyl-benzamide was prepared via a standard 1-(3-dimethylaminopropyl)-3-ethylcarbodiimide hydrochloride-mediated coupling between 5-amino-1-pentanol and p-tolylacetic acid in CH2Cl2 in the presence of triethylamine and 4-(dimethylamino)pyridine [42].

Representative Bead Encoding Procedure

Twenty dry macrobeads (˜3 mg solid supports) were placed in a 700 μl Eppendorf tube. A fresh 8.4 mM (in each tag) solution in dry CH₂Cl₂ was prepared in an oven-dried, Teflon-capped glass vial, and 50 μl of this tag solution was added to the Eppendorf tube. The tube was agitated for 45 min at room temperature on a tabletop orbital shaker. A 4.4 mg/ml solution of the catalyst, Rb₂(O2CC(Ph)₃)₄(5), in dry CH₂Cl₂ was prepared under Ar in an oven-dried, Teflon-capped glass vial, and 50 μl of the catalyst solution was added to the resin. The Eppendorf tube was agitated for 16 h (overnight) at room temperature. The supports were then washed in a 1 ml Bio-Rad tube 2×15 min CH2Cl2, 16 h (overnight) THF, 2×15 min THF, and 2×15 min CH₂Cl₂. Afterwards, the supports were dried under house vacuum for ˜15 min before proceeding to either another synthesis step or compound cleavage (as described above).

Representative Bead Decoding Procedure

Macrobeads were dried under house vacuum for at least 1 h prior to decoding. A 0.24 M solution of CAN in 5:1 THF/H2O was prepared (132 mg CAN/0.83 ml dry, degassed THF+0.17 ml double-distilled H2O) in an oven-dried vial. (Note: this solution should be prepared immediately before use.) A single macrobead was placed in a glass autosampler sample insert, and 5 μl of the CAN solution were added to the glass autosampler insert, followed by 8 μl of dry decane. The insert was centrifuged in a microfuge to separate the two layers before placing it into an autosampler vial which was capped tightly. The vial was sealed with Parafilm and heated at 37° C. for 21 h (in a standard laboratory incubator). After cooling the vial to room temperature, the glass insert was removed from the autosampler vial, and it was sonicated for 1-10 min. Once again, the insert was centrifuged to ensure separation of the two layers. Using a 200 μl Pipetman equipped with a gel-loading tip, the top decane layer was removed and transferred to a new GC autosampler glass insert. (After heating overnight, the CAN layer will be colorless, so caution must be used to avoid contamination of the decane layer with CAN in transfer.) A 1:1 BSA/decane solution in an ovendried vial was prepared. (Note: this solution should be prepared immediately before use.) 1.0 μl of this BSA solution was added to the decane layer in the GC insert which was then spun down in a microfuge for 30-40 s to ensure efficient mixing of the BSA solution with the sample. The insert was placed in an autosampler vial, capped tightly, and stored at 0° C. until GC analysis.

Supplementary Material

Bead stability studies, graphs of encoding/decoding optimization data, and details of the partial decoding of dihydropyrancarboxamide library 12 can be found at: http://slsiris.harvard.edu/home/research_results.html and are presented in part below.

### References for Example 1

[1] J. L. G. Nilsson, A. Carlsson, Dopamine-receptor agonist with apparent selectivity for autoreceptors: a new principle for antipsychotic action?, Trends Pharmacol. Sci. 3 (1982) 322-325.

[2] G. G. Borisy, E. W. Taylor, The mechanism of action of colchicine. Binding of colchincine-3H to cellular protein, J. Cell Biol. 34 (1967), 525-533.

[3] T. J. Mitchison, Towards a pharmacological genetics, Chem. Biol. 1, (1994) 3-6.

[4] S. L. Schreiber, Chemical genetics resulting from a passion for synthetic organic chemistry, Bioorg. Med. Chem. 6 (1998) 1127-1152.

[5] T. U. Mayer, T. M. Kapoor, S. J. Haggarty, R. W. King, S. L. Schreiber, T. J. Mitchison, Small molecule inhibitor of mitotic spindle bipolarity identified in a phenotype-based screen, Science 286 (1999) 971-974.

[6] G. MacBeath, A. N. Koehler, S. L. Schreiber, Printing small molecules as microarrays and detecting protein-ligand interactions en masse, J. Am. Chem. Soc. 121 (1999) 7967-7968.

[7] P. J. Hergenrother, K. M. Depew, S. L. Schreiber, Small molecule microarrays: covalent attachment and screening of alcohol-containing small molecules on glass slides, J. Am. Chem. Soc. 122 (2000) 7849-7850.

[8] S. L. Schreiber, Target-oriented and diversity-oriented organic synthesis in drug discovery, Science 287 (2000) 1964-1969.

[9] R. B. Merrifield, Solid phase peptide synthesis. I. The synthesis of a tetrapeptide, J. Am. Chem. Soc. 85 (1963) 2149-2154.

[10] A. Furka, F. Sebestyen, M. Asgedom, G. Dibo, General method for rapid synthesis of multicomponent peptide mixtures, Int. J. Pept. Protein Res. 37 (1991) 487-493.

[11] R. E. Dolle, Comprehensive survey of combinatorial library synthesis: 1999, J. Comb. Chem. 2 (2000) 383-433.

[12] K. C. Nicolaou, X. Y. Xiao, Z. Parandoosh, A. Senyei, M. Nova, Radiofrequency encoded combinatorial chemistry, Angew. Chem. Int. Ed. Engl. 34 (1995) 2289-2291.

[13] K. C. Nicolaou, J. A. Pfeierkom, H. J. Mitchell, A. J. Roecker, S. Barluenga, G.-Q. Cao, R. L. Affleck, J. E. Lillig, Natural product-like combinatorial libraries based on privileged structures. 2. Construction of a 10 000-membered benzopyran library by directed split-and-pool chemistry using NanoKans and optical encoding, J. Am. Chem. Soc. 122 (2000) 9954-9967.

[14] S. M. Steruson, J. B. Louca, J. C. Wong, S. L. Schreiber, Split-pool synthesis of 1,3-dioxanes leading to arrayed stock solutions of single compounds sufficient for multiple phenotypic and protein-binding assays, J. Am. Chem. Soc. 123 (2001) 1740-1747.

[15] R. A. Stavenger, S. L. Schreiber, Asymmetric catalysis in diversity-oriented organic synthesis: enantioselective synthesis of 4320 encoded and spatially segregated dihydropyrancarboxamides, Angew. Chem. Int. Ed. 40 (2001) 3417-3421.

[16] P. A. Clemons, A. N. Koehler, B. K. Wagner, T. G. Sprigings, D. R. Spring, R. W. King, S. L. Schreiber, M. A. Foley, A one-bead, one-stock solution approach to chemical genetics, part 2, Chem. Biol. 8 (2001) 1183-1195.

[17] D. S. Tan, M. A. Foley, B. R. Stockwell, M. D. Shair, S. L. Schreiber, Synthesis and preliminary evaluation of a library of polycyclic small molecules for use in chemical genetic assays, J. Am. Chem. Soc. 121 (1999) 9073-9087.

[18] E. Bayer, Protein synthesis, Angew. Chem. Int. Ed. Engl. 30 (1991) 113-129.

[19] B. B. Brown, D. S. Wagner, H. M. Geysen, A single-bead decode strategy using electrospray ionization mass spectrometry and a new photolabile linker: 3-amino-3-(2-nitrophenyl)propionic acid, Mol. Div. 1 (1995) 4-12.

[20] A. J. You, R. J. Jackman, G. M. Whitesides, S. L. Schreiber, Miniaturized arrayed assay format for detecting small molecule-protein interactions in cells, Chem. Biol. 4 (1997) 969-975.

[21] M. H. J. Ohlmeyer, R. N. Swanson, L. W. Dillard, J. C. Reader, G. Asouline, R. Kobayashi, M. Wigler, W. C. Still, Complex synthetic chemical libraries indexed with molecular tags, Proc. Natl. Acad. Sci. USA 90 (1993) 10922 10926.

[22] H. P. Nestler, P. A. Bartlett, W. C. Still, A general method for molecular tagging of encoded combinatorial chemistry libraries, J. Org. Chem. 59 (1994) 4723-4724.

[23] B. R. Stockwell, S. J. Haggarty, S. L. Schreiber, High-throughput screening of small molecules in miniaturized mammalian cell-based assays involving post-translational modifications, Chem. Biol. 6 (1999) 71-83.

[24] J. A. Tallarico, K. M. Depew, H. E. Pelish, N. J. Westwood, C. W. Lindsley, M. D. Shair, S. L. Schreiber, M. A. Foley, An alkylsilyl-tethered, high-capacity solid support amenable to diversity-oriented synthesis for one-bead, one-stock solution chemical genetics, J. Comb.Chem. 3 (2001) 312-318.

[25] T. Greene, P. G. M. Wuts, Protecting Groups in Organic Synthesis, 3rd edn., Wiley, New York, 1999.

[26] F. X. Woolard, J. Paetsch, J. A. Ellman, A silicon linker for direct loading of aromatic compounds to supports. Traceless synthesis of pyridine-based tricyclics, J. Org. Chem. 62 (1997) 6102-6103.

[27] Y. Hu, J. A. Porco, J. W. Labadie, O. W. Gooding, B. M. Trost, Novel polymer-supported trialkylsilanes and their use in solid-phase organic synthesis, J. Org. Chem. 63 (1998) 4518-4521.

[28] A. W. Czamik, Encoding methods for combinatorial chemistry, Curr. Opin. Chem. Biol. 1 (1997) 60-66.

[29] D. S. Tan, J. J. Burbaum, Ligand discovery using encoded combina-torial libraries, Curr. Opin. Drug Disc. Dev. 3 (2000) 439-453.

[30] S. P. Fodor, J. L. Read, M. C. Pirrung, L. Stryer, A. T. Lu, D. Solas, Light-directed, spatially addressable parallel chemical synthesis, Science 251 (1991) 767-773.

[31 ] S. Brenner, R. A. Lemer, Encoded combinatorial chemistry, Proc. Natl. Acad. Sci. USA 89 (1992) 5381-5383.

[32] J. Nielson, S. Brenner, K. D. Janda, Synthetic methods for the implementation of encoded combinatorial chemistry, J. Am. Chem. Soc. 115 (1993) 9812-9813.

[33] C. L. Brummel, I. N. W. Lee, Y. Zhou, S. J. Benkovic, N. Winograd, A mass spectrometric solution to the address problem of combinatorial libraries, Science 264 (1994) 399-402.

[34] S. K. Sarkar, R. S. Garigipati, J. L. Adams, P. A. Keiffer, An NMR method to identify nondestructively chemical compounds bound to a single solid-phase-synthesis bead for combinatorial chemistry applications, J. Am. Chem. Soc. 118 (1996) 2305-2306.

[35] E. J. Moran, S. Sarshar, J. F. Cargill, M. M. Shahbaz, A. Lio, A. M. M. Mjalli, R. W. Armstrong, Radio frequency tag encoded combinatorial library method for the discovery of tripeptide-substituted cinnamic acid inhibitors of the protein tyrosine phosphatase PTP1B, J. Am. Chem. Soc. 117 (1995) 10787-10788.

[36] A. R. Vaino, K. D. Janda, Euclidean shape-encoded combinatorial chemical libraries, Proc. Natl. Acad. Sci. USA 14 (2000) 7692-7696.

[37] M. A. McKervey, D. N. Russell, M. F. Twohig, Alkylation of benzene with K-diazoketones via cycloheptatrienyl intermediates, J. Chem. Soc. Chem. Commun. 8 (1985) 491-492.

[38] J. Adams, D. M. Spero, Rhodium(II) catalyzed reactions of diazocarbonyl compounds, Tetrahedron 47 (1991) 1765-1808.

[39] R. Johansson, B. Samuelsson, Regioselective reductive ring-opening of 4-methoxybenzylidene acetals of hexopyranosides. Access to a novel protecting-group strategy. Part 1, J. Chem. Soc. Perkin Trans. I (1984) 2371-2374.

[40] A. R. Vaino, K. D. Janda, Solid-phase organic synthesis: a critical understanding of the resin, J. Comb. Chem. 2 (2000) 579-596.

[41] R. E. Dolle, J. Guo, L. O'Brien, Y. Jin, M. Piznik, K. J. Bowman, W. Li, W. I. Ehan, C. Carvallaro, A. L. Roughton, Q. Zhao, J. C. Reader, M. Orlowski, B. Jacob-Samuel, C. D. Carroll, A statistical-based approach to assessing the fidelity of combinatorial libraries encoded with electrophoric molecular tags. Development and application of tag decode-assisted single bead LC/MS analysis, J. Comb. Chem. 2 (2000) 716-731.

[42] J. C. Sheehan, P. A. Cruickshank, G. L. Boshart, A convenient synthesis of water-soluble carbodiimides, J. Org. Chem. 26 (1961), 2525-2528.

[43] H. E. Blackwell, L. Pe\ddot{}rez, S. L. Schreiber, Decoding products of diversity pathways from stock solutions derived from single polymeric macrobeads, Angew. Chem. Int. Ed. 40 (2001) 3421-3425.

[44] J. S. Johnson, D. A. Evans, Chiral bis(oxazoline) copper(II) complexes: versatile catalysts for enantioselective cycloaddition, aldol, Michael, and carbonylene reactions, Ace. Chem. Res. 33 (2000), 325-335.

[45] K. A. Jorgensen, M. Johannsen, S. Yao, H. Audrain, J. Thorhauge, Catalytic asymmetric addition reactions of carbonyls. A common catalytic approach, Ace. Chem. Res. 32 (1999) 605-613.

[46] J. Coste, D. Lenguyen, B. Castro, PyBOP: a new peptide coupling reagent devoid of toxic byproduct, Tetrahedron Lett. 31 (1990) 205-208.

[47] A. B. Pangbom, M. A. Giardello, R. H. Grubbs, R. K. Rosen, F. J. Timmers, Safe and convenient procedure for solvent purification, Organometallics 15 (1996) 1518-1520.

Additional Information for Example 1

General Synthetic Methods

General Methods. Reagents were obtained from Aldrich Chemical Co., Acros, Novabiochem, or J. T. Baker and used without further purification. Reaction solvents (THF, Et₂O, DMF, toluene, and CH₂Cl₂) were obtained from J. T. Baker (HPLC grade) and purified by passage through two solvent columns prior to use. The CH₂Cl₂ and toluene purification systems are composed of one activated alumina (A-2) column and one supported copper redox catalyst (Q-5 reactant) column. The THF and Et₂O purification systems are composed of two activated alumina (A-2) columns, and the DMF purification system is composed of two activated molecular sieve columns. See: Pangborn, A. B.; Giardello, M. A.; Grubbs, R. H.; Rosen, R. K.; Timmers, F. J. Organometallics 1996,15, 1518-1520.

Diusopropylethylamine (DIPEA) and 2,6-lutidene were distilled from calcium hydride; MeOH was distilled from magnesium methoxide. Brominated polystyrene (Br-PS, 2 mequiv/g) was obtained from Polymer Labs (Product #:1462-9999, $18/g). Solution phase reactions were performed in oven- or flame-dried glassware under positive N₂ pressure.

Solid Phase Reactions. Small-scale solid phase reactions (5-10 mg resin) were performed in 500 μL polypropylene Eppendorf tubes with mixing provided by a Vortex Genie-2 vortexer fitted with a 60 microtube insert. Medium-scale solid phase reactions (20-500 mg resin) were performed in 2 mL fritted polypropylene Bio-SpinO chromatography columns (Bio-Rad) or 10 mL fritted polypropylene PD-10 columns (Pharmacia Biotech) with 360° rotation on a Barustead-Thermolyne Labquake™ Shaker. Large-scale solid phase reactions (>500 mg resin) were performed in silanized 50 or 100 mL fritted glass tubes equipped for vacuum filtration and N₂ bubbling. The tubes were silanized by treatment with 20% dichlorodimethylsilane/CH₂Cl₂ for 15 min, MeOH for 15 min, followed by oven heating at 120 ° C. for at least 2 h.

After small-scale reactions, resin samples were transferred to 2 mL BioSpin® columns. Resin samples in polypropylene columns were washed on a Vac-Man® Laboratory Vacuum Manifold (Promega) fitted with nylon 3-way stopcocks (Bio-Rad). Resin samples in glass tubes were washed in the tubes with alternating periods of N₂ bubbling and vacuum draining. The following standard wash procedure was used: 3×THF, 3×DMF, 3×THF, 3×CH₂Cl₂.

Resin samples were then transferred via spatula to 500 μL Ependorf tubes and suspended in Ar-degassed HPLC grade THF followed by pryidine and hydrogen fluoride-pyridine (Aldrich, HF(70%)/pyridine(30%)) in a ratio of 90:5:5. Samples were then sealed with parafilm and gently agitated on a vortexer for 30 min. Methoxy-trimethylsilane (TMSOME) was added and the samples were sealed with Parafilm and placed on a vortexer for an additional 30 min. The supernatant fluid was removed, transferred to another Eppendorf tube, and concentrated in vacuo.

Purification and Analysis. Flash chromatography was performed on E. Merck 60 230-400 mesh silica gel. TLC was performed on 0.25 mm E. Merck silica gel 60 F₂₅₄ plates and visualized by UV (254 nm) and cerium ammonium molybdate. HPLC was performed on a Nest Group (Southborough, Mass.) Hypersil C18 100 Å 3 μM, 4.6 mm×6 cm column using a flow rate of 3 mL/min and a 4 min gradient of 0-99.9% CH₃CN in H₂O/0.1% TFA, constant 0.1% MeOH with diode array UV detection. IR spectra were recorded on a Nicolet 5PC FT-IR Spectrometer or a Bruker Vector 22 Spectrometer with peaks reported in cm⁻¹. NMR spectra were recorded on Varian Inova 500 MHz and 400 MHz instruments. Solid-phase NMR spectra were recorded on a Varian Inova 500 MHz equipped with a Nanoprobe. ( (a) Fitch, W. L.; Detre, G.; Holmes, C. P.; Shoolery, J. N.; Keifer, P. A. J. Org. Chem. 1994, 59, 7955-7956. (b) Keifer, P. A.; Baltusis, L.; Rice, D. M.; Tymiak, A. A.; Shoolery, J. N. J. Magn. Reson., Series A 1996 119, 65-75.)

Chemical shifts are expressed in ppm relative to TMS (0.00 ppm) or residual solvents. Peak assignments were made based on homonuclear decoupling and/or two-dimensional DQF-COSY, TOCSY, and/or NOESY experiments. Mass spectra were obtained on JEOL AX-505H or SX-102A mass spectrometers by electron impact ionization (EI), chemical ionization (CI) with ammonia (NH₃), or fast atom bombardment ionization (FAB) with glycerol or 3-nitrobenzyl alcohol/sodium iodide (NBA/NaI) matrices. LC/MS data was obtained on a Micromass Platform LCZ mass spectrometer in atmospheric pressure chemical ionization (APCI) mode attached to a Waters 2690 HPLC system. LC/MS chromatography was performed on a Waters Symmetry C18 3.5 μM, 2.1 mm×50 mm column using a flow rate of 0.4 mL/min and a 10 min gradient of 15-100% CH₃CN in H₂O, constant 0.1% formic acid with 200-450 nmol detection on Waters 996 photodiode array detector. GC/ECD data was obtained on a Hewlett Packard 6890 Gas Chromatograph fitted with a 7683 series injector and autosampler, split-splitless inlet, μ-ECD detector, and a J&W DB1 15 m×0.25 mm×0.25 μm column. (Gradient start temperature: 110° C.; hold 1 min, ramp 45° C./min to 250° C., hold 2 min, ramp 15° C./min to 325° C., hold 2 min. Flow rate: constant flow, 1 mL/min. Inlet is purged at 1 min with flow rate 60 mL/min, reduced to 20 mL/min at 2 min).

Allyl Silane Linker Synthesis

Diisopropyl(4-methoxyphenyl)silane. A solution of p-bromoanisole (28.6 mL, 228 mmol, 1.0 equiv.) in THF (550 mL) was chilled to −78° C. (CO₂(s), acetone) and treated with n-BuLi (91.2 mL, 228 mmol, 2.5 M in hexanes, lequiv.) via cannula over a 5 min period. After 5 min a white precipitate began to form. The mixture had stirred for 30 min at −78° C. when diisopropylchlorosilane (34.6 g, 228 mmol, 1.0 equiv.) was slowly added via syringe. After 1 h the ice bath was removed, and the solution was allowed to come to 23° C. with continued stirring overnight. The mixture was treated with saturated NH₄Cl (50 mL) and extracted with ether (3×500 mL). The combined organic extracts were washed with brine, dried over MgSO₄, filtered and concentrated in vacuo to yield a light yellow oil. Silica gel chromatography (gradient: 3-5% EtOAc/hexanes) yielded (47.7 g, 94%) of a colorless oil. This material could also be purified by distillation [BP=76-85° C. @ 275 mTorr (40 g, 63%)]. TLC R_{f}=0.61 (1:9 EtOAc/hexane).s IR(film): 2393, 1853, 1710, 1691, 1658, 1584, 1482, 1346. ¹H NMR (500 MHz, CDCl₃): δ 7.48 (d, 2H, J=8.10), 6.95(d, 2H, J=8.10), 3.97(s, 1H, Si—H), 3.85 (s, 3H), 1.39(q, 2H, J=3), 1.10(d, 6H, J=6.5), 1.03(d, 6H, J=7.5). ¹³C NMR (126 MHz, CDCl₃): δ 137.13, 113.73, 113.62, 55.18, 18.95, 18.72, 11.08. Elemental analysis, Calcd.: C 70.21, H 9.97, Si 12.63. Found, C 70.43, H 9.83, Si 12.39.

Chloro(4-methoxyphenyl)diisopropylsilane. Diisopropyl(4-methoxyphenyl)silane (47.7 g, 214 mmol, 1.0 equiv.), was taken up in CH₂Cl₂ (700 mL). The solution was cooled to 0° C. and trichloroisocyanuric acid (16.6 g, 71.3 mmol, 0.33 equiv.) was carefully added in three equal portions, making sure that each portion had at least 7 min to react before the next was added. (Caution! Adding trichloroisocyanuric acid too rapidly results in a rapid evolution of gas and concomitant expulsion of the reaction vessel contents). The mixture was stirred at 0° C. for 40 min, followed by warming to 23° C. with stirring. The solids were filtered under an inert atmosphere, and the filtrate was concentrated in vacuo to yield 54.8 g (98%) of a cloudy oil. The chlorosilane, which is unstable, was used immediately and without purification in the next step.

Allyl(4-methoxyphenyl)diisopropylsilane. To the crude chloro(4-methoxyphenyl)-diisopropylsilane (54.8 g, 214 mmol, 1.0 equiv.) was added THF (335 mL) via cannula under Ar. The solution was chilled to 0° C. and treated with allylmagnesium chloride (128 mL, 256 mmol, 2.0 M in THF, 1.2 equiv.). After 3 h at 0° C., the solution was allowed to warm to 23° C. with stirring overnight (16 h). The mixture was treated with saturated NH₄Cl (50 mL), and the aqueous layer was extracted with ether (3×500 mL). The combined organic extracts were washed with brine, dried over MgSO₄, filtered, and concentrated in vacuo. The crude material was purified by silica flash chromatography (3-5% EtOAc/hexanes) to yield 52.86 g (94%) of a slightly cloudy, clear viscous oil. This reagent distills at 80° C. at 500 mTorr as a colorless oil. TLC R_{f}=0.40 (1:9 EtOAc/hexanes). IR(film): 2942, 2865, 1630, 1595, 1504, 1463, 1277. ¹H NMR (500 MHz, CDCl₃): δ 7.32 (d, 2H, J=6.84), 6.81(d, 2H, J=6.84), 5.82 (q, 1H, J=8.5, 8.5), 4.88 (d, 1H, J=17.05), 4.76 (d, 1H, J=9.77), 1.82 (d, 2H, J=7.32), 1.17 (q, 2H, J=7.3), 0.94 (d, 6H, J=7.3), 0.90 (d, 6H, J=7.3). ¹³C NMR (126 MHz, CDCl₃): δ 160.51, 136.48, 135.70, 125.78, 113.78, 113.62, 55.09, 19.34, 18.22, 18.17, 17.68, 11.30. Elemental analysis, Calcd.: C 73.22, H 9.98, Si 10.70. Found: C 73.25, H 9.97, Si 10.77.

Hydroboration of Allyl(4-methoxyphenyl)diisopropylsilane. Solid 9-BBN dimer (6.29 g, 53.0 mmol, 0.95 equiv.) was weighed out in a glove box and sealed under an Ar atmosphere. Freshly distilled THF (365 mL) and allyl(4-methoxyphenyl)diisopropylsilane (14.64 g, 55.8 mmol, 1.0 equiv.) were added via syringe, and the mixture was allowed to stir for 3 h at 23° C. The overall concentration of the allyl(4-methoxyphenyl)diisopropylsilane in THF was 0.16 M, which was the appropriate concentration for the subsequent Suzuki coupling. The yield of this reaction was assumed to be quantitative.

Suzuki Coupling. To the alkyl-borane containing THF solution above (53.0 mmol in 365 mL of THF, 1.74 equiv.) was added the solid Br-PS (15.25 g, 2 mequiv/g 30.5 mmol of Br, 1.0 equiv.) Care was taken to maintain an Ar blanket over the solution. Br-PS was allowed to swell for 45 min, and then treated with tetrakis(triphenylphosphine)palladium(0) (880 mg, 0.76 mmol, 0.025 equiv.) followed by aqueous NaOH solution (61 mmol, 30.5 mL of a 2M NaOH solution, 2.0 equiv.). The reaction was then heated to reflux with gentle stirring for 24 h. Pd(0) (880 mg, 0.76 mmol, 0.025 equiv.) was added, and the reaction was heated to reflux for another 12 h. The biphasic reaction mixture turned slightly green from its initial yellow color. The mixture was filtered, and the beads were washed repeatedly (see below). While it was unnecessary to agitate the beads during the wash cycle, it was critical to allow the beads sufficient time to absorb the washing solvent. Wash procedure: THF (2×100 mL×45 min), 3:1 THF/1 M NaCN (1×100 mL×1 h or until all dark color is gone), 3:1 THF/H₂0 (2×100 mL×45 min), 3:1 THF/IPA (2×100 mL×45 min), THF (2×100 mL×45 min), CH₂Cl₂ (2×100 mL×45 min). The beads were air-dried overnight, then placed on a lyophilizer for 24 h, producing an almost colorless, opaque resin. ¹H NMR (500 MHz, nanoprobe, CD₂Cl₂ gel phase): δ 7.34 (m, 4H), 6.82 (m, 4H), 3.69 (s, 3H), 1.76 (m, 2H), 1.22 (m, 2H), 1.16 (m, 2H), 0.97 (m, 2H), 0.91 (m, 12H). For a discussion of the effect of resin linker length on gel-phase NMR spectral linewidths, see: Keifer, P. A. J. Org. Chem. 1996, 61, 1558-1559. Elemental analysis: Found C 83.54, H 8.28, Si 4.35, Br<0.02, Cl 0.247.

Determination of Bead Loading by Elemental Analysis. 2.0 mmol p-bromopolystyrene beads, quantitatively loaded with the silicon linker above, contain 41 mg Si/g resin or 4.1% Si. Assuming quantitative loading, the mass of 1 g resin would increase to 1.37 g; therefore, the linker loading is calculated as 1.45 mequiv/mol. Thus, the resin loading is estimated from two elemental analyses parameters, % Si and % Br. The % Br<0.02 by weight indicates qualitative disappearance of Br (note that halogens can be confused by elemental analysis, hence it is necessary to perform separate Br and Cl analysis), while percent Si indicates the loading level. Percent Si typically ranges from 3.79 to 4.05%. The procedure used to calculate percent Si can overestimate the actual amount of Si by 0.2-0.3% as these numbers are calculated by weighing ash resultant from sample digestion with acid and residue combustion, which leaves some elements unresolved from Si. 4.35% Si is equivalent to 43.5 mg Si/g resin, or 1.54 mequiv Si/g. The actual loading used in subsequent calculations was 1.45 mequiv/g, the theoretical maximum. There were 9,350 beads/g of 500-600 copolymerizedp-bromopolystyrene beads with 2.0 mmol Br/g loading level. We assumed quantitative conversion, justified by disappearance of bromine and appearance of appropriate amount of silicon. Thus, the number of polystyrene beads in one gram of resin was then scaled with a 37% mass increase, or about 6,800 beads/g.

Library Encoding and Decoding Protocols

Representative Bead Encoding Procedure. Place 20 dry beads (approximately 3 mg resin) in a 700 μL Eppendorf tube. Prepare a fresh 8.4 mM (in each tag) solution in dry CH₂Cl₂ in an oven-dried, Teflon capped glass vial. (NOTE: The tag concentration can be cut by one-half to one-fifth, and the tags will still be readable by GC (the late tags will be weak). This might be necessary for large library syntheses where a large quantity of tag is required, or if more than 4 tags are used in each tagging step. Use the same volume of tag solution as described below.) Add 50 μL of the tag solution to the Eppendorf tube. Set the tube to shake for 45 min at room temperature on a tabletop orbital shaker. Prepare a 4.4 mg/mL solution of the catalyst, rhodium triphenylacetate (Rh₂(O₂CC(Ph)₃)₄), in dry CH₂Cl₂ under Ar in an oven-dried, Teflon capped glass vial. (NOTE: The catalyst concentration can be cut by one-half to one-fifth and the tags will still be readable by GC (the late tags will be weak). Use the same volume of catalyst solution as described below.) Add 50 μL of the catalyst solution to the resin and keep the Eppendorf in agitation for 16 h (overnight) at room temperature. Wash the resin in a 1 mL BioRad tube 2×15 min CH₂Cl₂, 16 h (overnight) THF, 2×15 min THF, and 2×15 min CH₂Cl₂. Dry the resin under house vacuum for ca. 15 min before proceeding to compound cleavage.

Compound Cleavage: Place the beads into a 700 μL Eppendorf tube. Add 100 μL of freshly-prepared 5% (HF/py)/THF solution (v/v). Set the tube to shake for 90 min at room temperature on a tabletop Eppendorf shaker. Quench HF by adding 200 μL TMSOMe to the tube. Set the tube to shake for 30 min at room temperature on a tabletop Eppendorf shaker. Collect the filtrate (if desired) and wash the resin: 3×5 min CH₂Cl₂, 3×5 min THF, and 3×5 min CH₂Cl₂. Dry under house vacuum for at least 1 h before decoding.

Representative Bead Decoding Procedure. Place one bead into an autosampler glass sample insert with the aid of tweezers. A 0.24 M solution of CAN in 5:1 THF/H₂O is prepared (132 mg CAN/0.83 mL dry, degassed THF +0.17 mL doubly-distilled H₂0) in an oven-dried vial. This solution should be prepared immediately before use. Add 5 μL of the CAN solution to the glass autosampler insert. Add 8 μL of dry decane to the glass insert and then centrifuge the insert in a Micro-Centrifuge to separate the two layers. Place the insert in an autosampler vial and cap tightly. Seal with Parafilm, and heat the glass insert at 37° C. for 21 h (in a standard laboratory incubator). Allow the sample to cool to room temperature, and remove the glass insert from the autosampler vial. Sonicate the insert for 1-10 min. Centrifuge the insert again in the Micro-Centrifuge. Use a 200 μL Pipetman equipped with a gel-loading tip to remove the top decane layer and transfer it to a new GC autosampler glass insert. (After heating overnight, the CAN layer will be colorless, so caution must be used to not contaminate the decane layer with CAN in transfer.) Prepare a 1:1 BSA/decane solution in an oven-dried vial. This solution should be prepared immediately before use. Add 1.0 μL of this BSA solution to the decane layer in the GC insert. Spin down the insert in the Microfuge for 30-40 sec to ensure efficient mixing of the BSA solution with the sample. Place the insert in an autosampler vial, cap tightly, and store at 0° C. until GC analysis.

### Example 2

Development of A High-Throughput Synthesis and Screening Platform:Bead Arraying; Compound Cleavage, Elution, and Resuspension; Assay Format, Design, and Annotation.

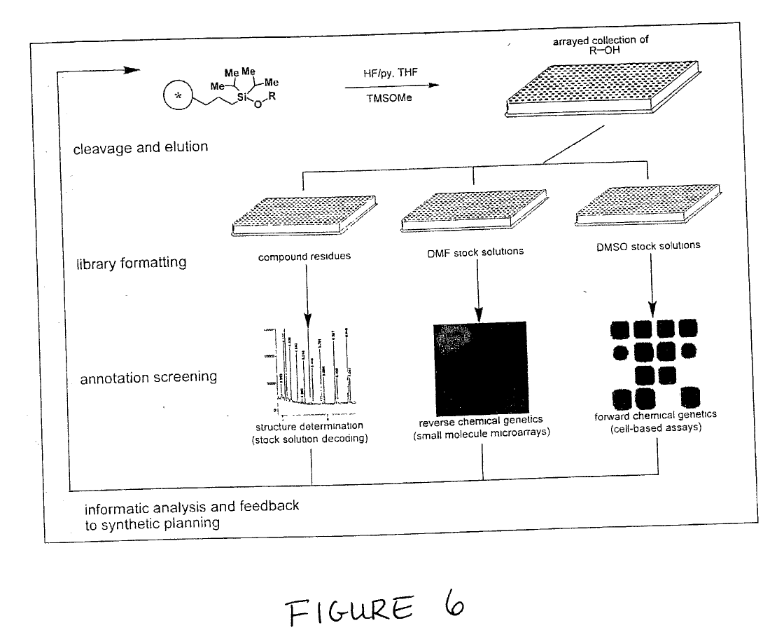

Example 1 described the first phase of development of a two-part ‘one-bead, one-stock solution’ technology platform, including the scaled synthesis of a high-capacity solid-phase bead/linker system and the development of a reliable library encoding/decoding strategy. This example describes the second phase of development of this platform, including (1) bead arraying, (2) automated compound cleavage, elution, and resuspension as spatially arrayed stock solutions, and (3) assay format, design, and annotation. FIG. 6 shows an overview of these aspects of the invention.

Systematic Library Formatting and Annotation

A major objective of our technology platform is to deliver quantities of compounds from single beads that are compatible with numerous chemical genetics assays. In general, we perform fluoridolysis to cleave silyl ether-linked small molecules from our trialkylsilyl linker system as described herein and in [14,15], and subsequently elute released compounds trapped in the interior of 500-600 μm PS beads. To optimize the conditions for compound release, we elected first to optimize the cleavage reaction and compound elution steps manually, allowing us to avoid extensive reprogramming of our robotics workstations to handle the large number and range of experimental variables. To ensure standardization of these experiments, we prepared three large batches of test resin that required minimal handling prior to compound loading. Using “off-the-shelf” beads prepared as described in Examples 1 and 4 and in [14, 15], we activated diisopropylalkylsilyl-functionalized resin 1 containing ˜200 nmol of Si/bead by treatment with excess triflic acid (TfOH) [16] to form 2, then trapped primary, secondary, and phenolic alcohols 6-8 in the presence of excess 2,6-lutidine to generate silyl ethers 3-5 as shown in the scheme below.

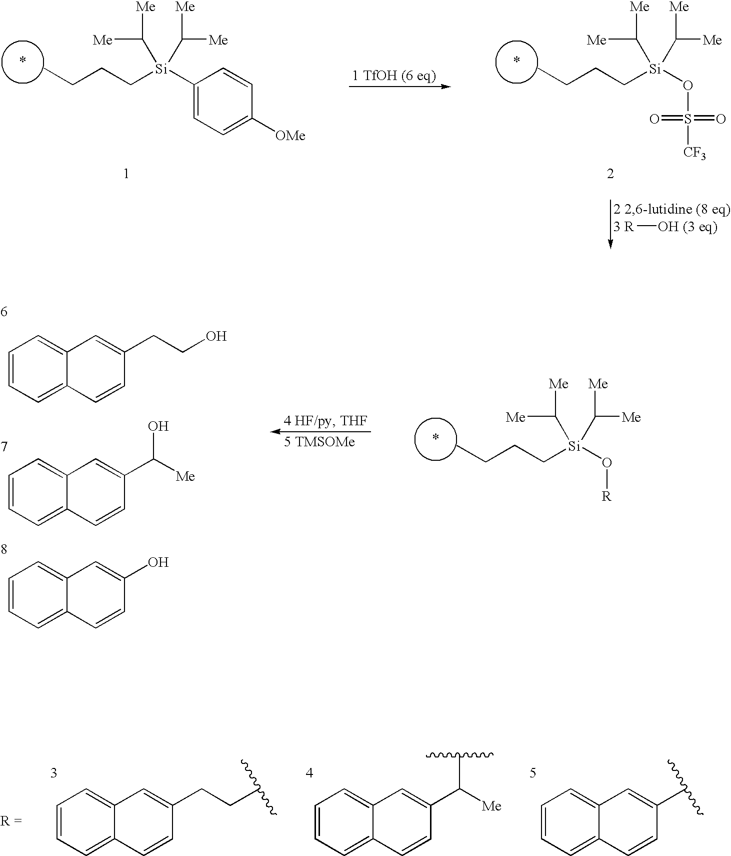

Naphthyl derivatives 6-8 were chosen on the basis of their ease of quantitation by ultraviolet (UV) spectrophotometry and high-performance liquid chromatography (HPLC). To facilitate quantitative measurements of yield from individual PS beads, we first used pure samples of 6-8 to establish standard curves of UV peak area (224 nm) versus concentration for 6-8 using our HPLC system. In addition, we performed mock cleavage reactions on 3-5 to determine the lower limit of detection. For each model alcohol, we can quantitatively detect 1-30 nmol of injected compound using our instrumentation.

Optimization of Compound Cleavage Conditions

Fluoride is a preferred reagent for the cleavage of silyl ethers, resulting in fluoridolysis of the Si-O bond and generation of the corresponding silylfluoride. Previously, we have cleaved compounds from 1 primarily with hydrogen fluoride/pyridine (HF/py) solution [14,15,20], a standard reagent for fluordolysis [19] that is available in a commercial preparation. Excess HF in cleavage reactions is quenched with methoxytrimethylsilane (TMSOMe) [21], resulting in the production of volatile by-products (methanol and trimethylsilylfluoride) that are easily removed from the mixture under vacuum. Before optimizing the cleavage reaction itself, we determined how quickly the addition of TMSOMe results in the quenching of excess HF. To our knowledge this has not been tested explicitly, and is important to establish the timing of an automated cleavage and elution process.

We performed 19 ^{F}NMR experiments on simulated cleavage reaction mixtures. We added 20 μl of a tetrahydrofuran (THF) solution containing 5% (v/v) HF/py and 5% (v/v) additional pyridine to individual wells of a 384-well plate, each containing a single bead from resin batch 113. Each reaction mixture was treated with 20 μl of either TMSOMe or n-Bu2O, and immediately transferred to a lined glass NMR tube for determination of the ⁹¹FNMR spectrum. The spectrum of samples treated with TMSOMe consists of a singlet at N=52.4 ppm, which corresponds exactly to the singlet in the ¹⁹F NMR spectrum of an authentic solution of trimethylsilylfluoride in THF. n-Bu2O was chosen as a control because its density (0.764 g/ml) is similar to that of TMSOMe (0.756 g/ml). The spectrum of samples treated with n-Bu2O consists of a broad singlet at N=125.3 ppm, and is identical to the spectrum of an authentic solution of HF/py alone. We conclude that HF/py is quenched within 5 min of the addition of TMSOMe as described. In addition to the timing of cleavage and elution, this experiment establishes an important safety margin for the handling, especially robotically, of microtiter plates containing quenched HF/py solutions.

To our knowledge no solid-phase study has been performed using a standardized system that addresses all experimental variables for HF/py-mediated cleavage of resin-bound silyl ethers We systematically tested the composition of the cleavage cocktail, including the identity and concentration of the cleavage reagent, as well as the kinetics of the cleavage reaction. Although these optimization experiments were performed manually, we sought to emulate closely the experimental conditions we envisioned for automated cleavage of compounds. Cleavage experiments were carried out manually by arraying individual beads from resin batches 3-5 into single wells of 384-well microtiter plates. At this stage, all exposures of beads 3-5 to cleavage reagents took place in 20 μl pal of THF solution containing a cleavage reagent and a buffering base, as required. After addition of the cleavage cocktail, plates were covered by an empty microtiter plate to prevent evaporation and allowed to incubate at room temperature for the duration of the experiment. Individual cleavage reactions were quenched by addition of 20 μl TMSOMe for an additional 10 min. After evaporating quenched reaction mixtures, we eluted products 6-8 by washing each bead twice with 20 μl acetonitrile (CH₃CN) and pooling the two eluates. Calculation of per-bead yield was performed by HPLC analysis as described above.

To select a cleavage reagent, individual beads from resin batches 3-5 were exposed for 30 min to a 5% (v/v) solution in THF of either HF/py, HF/triethylamine (HF/Et₃N), aqueous HF, or tetrabutylammonium fluoride (TBAF). In addition, three buffered cocktails were tested: HF/py buffered with 20% additional pyridine, HF/Et₃N buffered with 20% additional Et₃N, and aqueous HF buffered with 20% 2,6-lutidene. The data suggest that HF/py is four times as effective as any other reagent in releasing 6, all other factors being equal. Similar results were obtained with 25 secondary alcohol 7, though with significantly reduced yields. While not wishing to be bound by any theory, we believe this is partially due to a lower loading level for 4, and partially due to the short time of exposure, a difference previously observed between primary and secondary silyl ethers [19]. Notably, TBAF performed as well or better than any of the acidic fluoride reagents in the release of 8. Furthermore, for each HF reagent tested, cleavage of 5 to yield 8 was 30 markedly enhanced in the presence of additional buffering base. Taken together, these data suggest that HF/py, buffered with additional pyridine, is the most general cleavage reagent for this system. While not wishing to be bound by any theory, we suggest, however, that TBAF, or HF solutions buffered with bases other than pyridine, might prove slightly more useful for cleaving phenolic silyl ethers.

Having chosen HF/py as a cleavage reagent, we tested whether the amount of additional buffering pyridine was important to the yield of the cleavagereaction. Once again, this parameter proved sensitive to the type of silyl ether linkage. For resin 3, loaded with primary alcohol 6, no signi¢cant diᵢerence was detected up to 20% additional pyridine (20.0±3.2 nmol/bead). However, both resin 4 and resin 5 were sensitive to the amount of buffering base. Resin 4 showed a marked decline in the yield of 7 as buffering pyridine was increased from 5% (3.61±0.3 nmol/bead) to 20% (2.0±0.3 nmol/bead). In contrast, but consistent with our earlier observation, resin 5 showed a monotonic increase in the yield of 8 as buffering pyridine was increased from 5% (24.3±2.6 nmol/bead) to 20% (30.3±0.6 nmol/bead). Furthermore, in the absence of buffering pyridine, the yield of 8 was only half-maximal (14.1±6.1 nmol/bead). Because our HPLC eluent contains trifluoroacetic acid, we believe this effect is indeed a consequence of poorer cleavage, rather than an increase in absorbance of 8 due to deprotonation at the detection step. For generality, we used 5% buffering pyridine in all further experiments. In no case were yields using this cocktail lower than 80% of the maximal yield in any parallel buᵢering experiment. It should be noted, however, that additional amounts of buffering pyridine might significantly improve the release of phenolic silyl ethers.

We tested whether decreasing the amount of HF in the cleavage reaction would compromise compound yields. Since HF, even in solution, is both corrosive and highly toxic, it would be prudent to use the minimum acceptable concentration, especially in robotic implementations. To test this parameter, resins 3-5 were exposed for 120 min to varying concentrations of HF/py, each buffered with 5% additional pyridine, in THF. Not surprisingly, all lower concentrations of HF resulted in lower yields for all three model compounds. Additional experiments with higher concentrations of HF/py suggested that little additional yield was accessible by raising the HF concentration, though 10% HF/py did increase the rate of cleavage of 4 to 7 modestly. Thus, a THF solution containing 5% commercial HF/py, plus 5% additional pyridine (HF/py+py), proved most general for liberating 6-8 from trialkylsilyl linker 1.

Despite the optimization of our cleavage cocktail, the absolute yields in the experiments described above fall short of our goal of 50 nmol/bead. To improve these yields, we subjected individual beads from resin batch 3 to our ‘best practices’ cleavage cocktail for increasing reaction times. In contrast to our past experience [9,14], this demonstrated that the release of alcohol 6 proceeds well past the 120 min mark in the cleavage reaction. Indeed, we judged the reaction to be complete only after 300 min, as evidenced by the fact that an overnight reaction did not further increase the yield. This optimization step proved important to achieving a high yield of 6 (>100 nmol) from large PS beads. To test the possibility that multiple exposures to HF/py, presumably with an intervening elution step [22], might also increase the yield of silyl ethers cleaved from solid support we first exposed resins 3-5 to HF/py+py cocktail for 300 min, after which compounds 6-8 were eluted normally (2×20 WI CH₃CN) from the beads. Next, the same beads were re-subjected to fresh cleavage cocktail. Eluates from the first and second exposures were analyzed separately by HPLC. The results show that indeed some residual compound remains attached to the beads following the first exposure to cleavage cocktail. Elution experiments (see below) support the notion that this observation is not due to incomplete elution of material that has been covalently cleaved. However, since the quantities involved are small relative to the first exposure (˜10%), and owing to the intrinsic hazards of working with HF solutions, we elected not to employ multiple exposures as part of our general method-ology. Rather, we use a single 300 min exposure to a cocktail of HF/py+py in THF as our protocol for cleaving compounds from trialkylsilyl linker 1. In some cases, particularly with secondary and phenolic silyl ethers, adjustments to this protocol are warranted. In general, our data reflect these trends and may be used as guidelines in formatting libraries of specific compounds.

Optimization of Compound Elution Conditions

Following evaporation of the by-products of quenched cleavage reactions, library compounds must be eluted from the PS support. Even less work has been directed at elution studies than at compound cleavage, particularly for large PS beads. To address compound elution from large PS beads systematically, we performed a series of optimization experiments analogous to those described above, using 3 as the model substrate. We chose five solvents to compare directly their ability to elute. cleaved compounds from the PS matrix: dichloromethane (CH₂Cl₂), CH₃CN, dimethylformamide (DMF), DMSO, and THF. Individual beads from resin batch 3 were exposed to our “best practices” cleavage cocktail for 180 min, reactions quenched with TMSOMe, and the solvent and volatile by-products removed by evaporation. Compound 6 was then eluted from each bead by two successive 20 μl washes in one of the solvents, which were pooled for HPLC analysis. The results demonstrated that CH₃CN and DMF are superior to the other three solvents at eluting 6. Notably, DMSO performed half as well as either CH₃CN or DMF, while THF gave yields less than 10% those of CH₃CN or DMF. As there is no significant difference in yield between CH₃CN- and DMF-based elution, we suggest that either solvent might be used for e¤cient elution from large PS beads.

To test whether multiple iterations of elution would serve more effectively to extract compound 6 from the resin, we exposed individual beads from batch 3 to HF/py+py cocktail for 180 min, quenched, and evaporated. Next, we subjected each bead to six successive rounds of elution with 20 μl CH₃CN. Rather than pooling these eluates as before, we kept each 20 μl aliquot separate in order to compare the yield in the first elution with that in each subsequent elution. As we expected, most of the compound (59%) was released during the first elution iteration. However, a significant portion of 6 emerged during the second (28%) and later (9% total) elutions steps, suggesting that pooling multiple elutions from large PS beads is effective in generating stock solutions containing large quantities of compound. As the iterations proceed, the signal from successive elution steps drops below the threshold for HPLC detection. When these data are weighted by their individual signal-to-noise ratios, and the results plotted as a cumulative percent elution across all six iterations, it is evident that maximal elution (96.2%) is accomplished after four elution iterations.

Finally, we explored whether increasing the length of time spent in each cycle of CH₃CN elution would improve the elution of 6. For comparison, all previous experiments involved the addition and immediate withdrawal of solvent, such that the soak time in elution solvent was <3 s. Here, replicate beads from batch 3 were independently cleaved as before, and each bead was subjected to four 20 μl elution cycles in CH₃CN for 1 min, 3 min, or 10 min. Surprisingly, this optimization provided the largest overall improvement in absolute yield. With 1 min soak times in each cycle, an average of 60% more of 6 was removed from the bead than without waiting. On average, 109% more of 6 was eluted with 10 min soak times. Similar experiments, in which resin 4 or 5 was subjected to cleavage and then eluted with 10 min soak times, likewise gave increased average yields of 7 (177%) and 8 (52%), respectively. In summary, optimization of cleavage and elution parameters resulted not only in the identification of a general protocol for releasing compounds from trialkylsilyl linker 1, but also in a several-fold improvement of total compound yield. Furthermore, we have uncovered several trends that will aid in formatting libraries that are linked to the solid phase through secondary or phenolic silyl ether linkages. Our optimization procedure proceeded iteratively, such that optimal parameters found in initial experiments were used in subsequent experiments. Consequently, we can readily plot the increase in overall yield across the series of manual optimization experiments.

Robotic Implementation of Cleavage and Elution

In order to implement the cleavage and elution protocol robotically, a number of additional challenges had to be surmounted. We wished to first array individual beads from the diversity-oriented synthesis, one bead per well, into 384-well microtiter plates. Next, we wished to expose each bead successively to the cleavage cocktail, the quenching reagent, and multiple instances of an elution solvent (CH₃CN or DMF). Throughout this procedure, the positional integrity of the beads and corresponding stock solutions must strictly be maintained. In other words, each bead must remain associated with exactly one well in both the cleavage plate and the “mother plate” into which successive elutions are pooled.

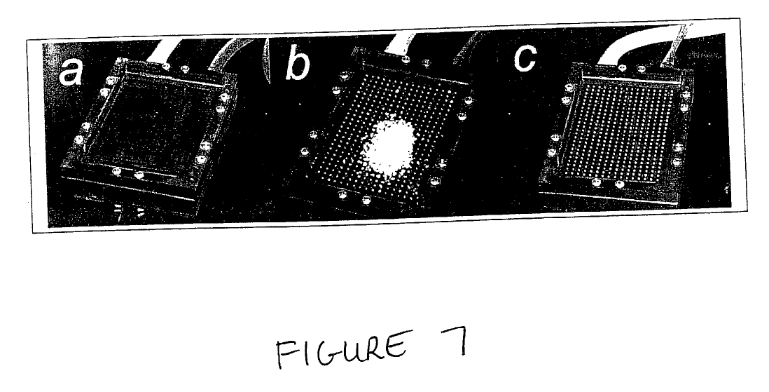

To address the problem of bead arraying we have optimized the use of a bead arrayer that immobilizes 384 beads in an equal number of depressions. Operation of the bead arrayer is shown in FIG. 7. The arrayer is designed for connection to both a standard vacuum line and a standard nitrogen line. Beads are trapped by application of vacuum to the apparatus, so that excess beads (FIG. 7b) can be brushed away easily and recovered, leaving exactly 384 beads on the arrayer in a regular 24×16 matrix (FIG. 7c). To deposit individual beads into the wells of a microtiter plate, the arrayer is inverted onto the plate, such that one bead is suspended over each well of the plate, and the vacuum relieved by pressurization with nitrogen. Beads are thus pushed out of the depressions and down into the wells of the microtiter plate. In practice, we have found that pre-wetting the wells with a small portion of THF or CH₃CN (10 μl/well, delivered by a bench-top plate filler) dramatically simplifies this procedure. Dry beads tend to cling to the walls or lip of a well due to static electricity. Pre-wetting the wells with solvent serves to trap the bead at the bottom, which greatly eases subsequent plate handling. Furthermore, when done just prior to adding cleavage cocktail, a portion of THF serves to pre-swell the resin, which modestly enhances the rate of the cleavage reaction.

Preferably HF/pyis not dispensed using conventional liquid handling robots due to the highly corrosive nature of this reagent. At issue are both the capture of toxic vapors, to minimize hazards to both people and equipment, and the composition of materials that come into direct contact with HF/py solutions. The first of these issues was addressed by encasing our robotic library formatting station in a specially designed ductless fume hood equipped with a recirculating air filtration system. In particular, this hood provides adequate ventilation of the station, as well as protective shields that isolate the operator from the robotics during operation. To dispense HF/py safely, we used a ceramic pump system coupled to both a rectilinear dispenser head (fitted with HF-resistant tubing) and a standard plate-stacker to deliver microtiter plates to the dispenser platform. We use the same instrumentation to deliver TMSOMe to cleavage reactions, which has the added benefit of quenching the pumps and tubing, converting any residual HF into non-corrosive volatile byproducts. To evaporate quenched cleavage reactions, we use a temperature-controlled vacuum centrifuge equipped with microtiter plate adapters. To elute compounds from the beads, we use a syringe-array liquid handling robot coupled to another plate-stacker. This combination is able to eject compound elution and pooling of stock solutions into mother plates at a rate of 4-6 plates/h. To validate the portability of our optimized cleavage and elution protocol to this robotics environment, we arrayed 384 beads from each of batches 3-5 into three microtiter plates using the bead arrayer. Each plate was subjected to treatment with our HF/py+py cocktail, delivered robotically, followed by TMSOMe after 300 min (or ethoxytrimethylsilane). Following evaporation of quenched reactions, each bead was subjected to four 10 min elution iterations, each in 20 μl CH₃CN, which were pooled into fresh 384-well “mother plates”. Aliquots from 10 wells of each plate were subjected to HPLC analysis. This work illustrates the success of robotic implementation of our strategy using model compounds. For each of 6-8, the robotic cleavage and elution protocol adequately reproduces the same experiments carried out manually. In all cases, the absolute yield of 6-8 is greater than our goal of 50 nmol/bead.

Cleavage and Elution of a Diversity Set of Dihydropyrancarboxamides

To demonstrate that the optimized robotic cleavage and elution protocol can deliver actual library members from diversity-oriented syntheses into chemical genetic assays, we used an encoded, split-pool library of 4320 dihydropyrancarboxamides (10), whose development and synthesis are described in detail in [27]. Briefly, this library consists of three diversity-generating steps, the first two of which were encoded with chloroaromatic tags as described in Example 1. As the final diversity-generating step was not chemically encoded, we acquired this library as 54 separate portions of dry resin (9) totaling three theoretical copies of 4320 stereochemically and structurally distinct compounds (10). We first exposed 324 individual beads, six from each of the 54 separate portions of 9, to our manual “best practices” cleavage and elution conditions (FIG. 7) in a single microtiter plate. In this case, compounds were eluted directly into DMF to prepare a diversity plate of stock solutions (plate 0) amenable to small molecule printing.

Glass microscope slides were activated for covalent attachment of alcohols, and compounds (10) from the 320 stock solutions were printed as described previously [3]. To test the availability of 10 to a protein-binding assay, we probed the small molecule microarray with purified Cy5-labeled (His)6-FKBP12 [2]. As a positive control for protein-ligand interaction, AP1497 [24,25] was included on the slide by adding it in DMF solution to an empty well of the stock plate. Following incubation, the slide was washed and scanned for the presence of a Cy5 fluorescence signal [2], which appeared both at the AP 1497 control spots and at spots corresponding to a member of 10. The bead corresponding to the novel FKBP 12-binding entity was subjected to the optimized bead decoding protocol described in Example 1. Using this procedure, we were able unambiguously to determine the structure of this “hit” (11) in a protein-binding assay, as was subsequently confirmed by tandem liquid chromatography/mass spectroscopy (LC/MS).

Formatting and Assaying of Representative Dihydropyrancarboxamides

To apply the robotic process to a fraction of resin 9, we arrayed 128 beads from each of three separate portions of 9 into a single 384-well microtiter plate. These beads were subjected to robotic cleavage and CH3CN elution as described earlier to prepare a “mother plate” (plate 1) containing 384 members of 10. Subsequently, the “mother plate” was mapped into six “daughter plates” by volumetric transfer using the syringe-array robot. “Daughter plates” were prepared for cell-based assays [1,8] (50% of stock solution), HPLC analysis (25%), LC/MS analysis (10%), small molecule printing [2,3] (2×5%), and stock solution decoding (5%). In each case, the CH3CN solution was evaporated following volumetric transfer so that each copy could be resuspended in the solvent most appropriate to its use. In particular, DMSO was used to resuspend the “daughter plate” for cell-based assays and DMF was used to resuspend the ‘daughter plate’ for small molecule printing. The plate containing the beads was also stored, but due to the success of stock solution decoding [28], and the difficulties associated with maintaining positional integrity within plates of beads, formatting a “daughter plate” explicitly destined for structure determination has become the standard in our library realization process.

Both plates of stock solutions (10) were used in phenotypic assays. In particular, we exposed living human A549 lung carcinoma cells to 708 (324+384) stock solutions under two different assay conditions. These experiments were performed with a hand-held pin-transfer tool, though our complete technology platform includes a pin-transfer robot capable of mapping into multiple microtiter plates. In general, cultured cells exposed to 5-bromodeoxyuridine (BrdU) will incorporate this base analog into their DNA when actively dividing, and this incorporation can be detected by cytoblot assay using antibodies directed against BrdU [8]. First, to determine if any stock solution of 10 inhibits BrdU incorporation, we transferred ˜100 nl of each stock solution into individual assay wells containing A549 cells actively growing in the presence of 1% fetal bovine serum. Second, we exposed A549 cells to ˜100 nl of each stock solution, and simultaneously challenged the cells with 100 μM genistein, a broad-spectrum protein tyrosine kinase inhibitor [26]. Under the latter conditions, BrdU incorporation, again judged by cytoblot assay [8], is impaired. Thus, “hits” in the former assay are detected as a loss of signal in a high-signal array, while “hits” in the latter assay are detected as a gain of signal in a low-signal array. The latter assay is referred to as a genistein suppressor screen, as we are seeking a member of 10 that can suppress the ability of genistein to inhibit BrdU incorporation.

For each of these assays, aliquots from each of the two plates (10) were exposed to cells in duplicate to ensure the fidelity of the results. Compounds were scored as “hits” only if they scored strongly in both replicates of a given experiment. From plate 0, 11 compounds scored as inhibitors of BrdU incorporation, while 10 compounds scored as suppressors of the action of genistein. From plate 1, 12 compounds scored as inhibitors of BrdU incorporation, while nine compounds scored as suppressors of the action of genistein. It is interesting that roughly the same number of first-pass “hits” were identified on each plate, despite the difference in diversity between the two collections. This finding may reflect the fact that assay results were tabulated by visual scoring of photograpic film, but is not limited to such detection methods. Conversely, in the case of an FKBP12-binding assay using microarrayed compounds, plate 1 produced no “hits”. To ensure that we can obtain exact structural information on the “hits” found in these experiments, we performed either bead decoding [15] or stock solution decoding as described in Example 4 on all 42 compounds scoring as positive in either assay. Decoding results were compared with LC/MS results for each sample to verify that a compound of the correct mass was present. In all but nine cases, LC traces revealed a single clean peak, and for each of the 42 “hits”, a parent ion or fragment matching the proposed structure was observed by MS. Thus, we were able to decode and confirm the structure of each “hit” detected in either the BrdU or the genistein suppressor cytoblot assay.

From a statistical perspective, the library of dihydropyrancarboxamides (9) was fully encoded, either chemically using chloroaromatic tags (first two diversity-generating steps), or positionally by inclusion into one of 54 pools of resin (third diversity-generating step). Our collection of decoded “hits” was analyzed to assign statistical significance to a process of “codon” selection, by a given assay, of particular encoded events (or combinations of encoded events) during the chemical history of the library. One immediate consequence is that a consensus set of structures corresponding to a particular assay activity need not be limited to individual structures that scored as “hits” in the assay. For example, if two codons corresponding to building blocks from two different diversity-generating steps were each strongly selected by a given assay, one might predict that a compound incorporating both moieties would yield higher potency in that assay. In the absence of additional information, we would predict such a consensus structure even if the exactcompound in question was not present in the initial screen. Alternatively, if the assay in question selected against this particular combination of codons, we would uncover this “forbidden” combination, even if each codon alone was frequently observed among structures scoring as “hits”. Traditionally, structure-activity relationships are determined by processes ranging from an intuitive viewing of “hit” structures to a comparison of “hits” on the basis of existing quantitative molecular descriptors (each based on some arbitrary metric). Our analysis introduces a novel approach, whereby we require no structural information in advance of defining significant biological activity. Rather, we allow the biological system under study to dictate the requirements for its activity. Such analysis illustrates the power of annotation screens to inform chemistry, through the technology platform, in ways that can influence planning steps in future diversity-oriented syntheses.

This Example described the second phase of development of our technology platform. The platform consists of, in part, an optimized procedure for compound cleavage and elution from large PS beads, a novel bead arraying method, and robotic implementation of library formatting, the process by which small molecules from diversity-oriented syntheses are made accessible to chemical genetic assays. We validated this approach by successfully synthesizing, encoding, and formatting a split-pool library of dihydropyrancarboxamides (9). It is important to note that optimization of the library formatting process occurred independently of the development of chemistry required to synthesize the library. Rather, optimization of the formatting process used generic model compounds to establish parameters, while formatting the split-pool library used the output of the optimization as a general, or “best practices”, method for library realization.

By exposing each member of a diversity-oriented synthesis to multiple phenotypic and proteomic assays, we can annotate each compound in the collection in a way that is complementary to other methods of small molecule characterization, such as MS and NMR. Statistical analysis of the biological performance of an encoded collection of small molecules allows us to inform further synthetic efforts (e.g. scaled synthesis of subset libraries based on primary screening data) in ways not necessarily available by traditional structure-activity analyses. Annotation screening is a term we use to describe the generation of multiple datasets by comprehensive screening of such libraries over a range of biological outcomes.

Materials and Methods

Model resin preparation Compounds 6 (2-naphthaleneethanol), 7 (α-methyl-2-naphtha lenemethanol), and 8 (2-naphthol) were obtained commercially (Sigma-Aldrich) and dried azeotropically prior to the loading reaction. Resin 1 was prepared as described in Example 3, and contains ˜200 nmol Si/bead calculated based on elemental analysis, assuming that 550 μm is the average bead size in a population of beads pre-sized at 500-600 μm. Loading reactions were performed in flitted polypropylene PD-10 columns (Amersham Pharmacia Biotech) and agitated by rocking on a Labquakel (Barnstead Thermolyne) shaker. Resin samples were washed on a Vac-Man® vacuum manifold (Promega) fitted with nylon stopcocks (Bio-Rad). HPLC-grade reaction solvents (J. T. Baker) were purified by passage through two solvent columns prior to use. Et₃N and 2,6-lutidene were distilled over calcium hydride. In loading reactions, bromostyrene-copolymerized beads were added to a PD-10 (Amersham Pharmacia Biotech) column, which was capped with a septum and plastic stopcock and flushed with Ar. After swelling with CH₂Cl₂ (10 ml), a 2.5% (v/v) solution of TMSCl in CH₂Cl₂ was added. The beads were suspended for 15 min and filtered with Ar pressure. The beads were washed with CH₂Cl₂ (3×2 min), then suspended in a solution of TfOH (6 eq.) in CH₂Cl₂ for 15 min, during which time Ar was bubbled gently through the reaction via a syringe. Next, the beads were rinsed with CH₂Cl₂ (3×2 min) under Ar and suspend in CH2Cl2. Freshly distilled 2,6-lutidine (8 eq.) and model alcohol 6, 7, or 8 (3 eq.) were successively added. The tube was capped and sealed to stand for 18 h at ambient temperature, after which the beads were foltered and rinsed with CH₂Cl₂ (4×3 min) and dried under house vacuum.

Cleavage and Quenching

Commercially available HF/py (Sigma-Aldrich) is approximately a 7:3 mixture of HF and pyridine, which was buffered with additional pyridine in THF solution. In manual experiments, beads were transferred individually by forceps to wells of 384-well microtiter plates (Genetix). Cleavage and quenching reagents, as well as elution solvents, were added by a P20 single-channel pipettor (Gilson). Data from ¹⁹F NMR experiments were obtained at 470.169 MHz on a Varian (Varian, Inc., http://www. varianinc.com/) AS500 (nt=128). To avoid etching of the NMR tube by HF/py solutions, samples were placed in a PTFE-FEP NMR tube liner (Wilmad-LabGlass).

HPLC Quantitation

HPLC analysis was carried out using a ThermoSeparation Products (Thermo-Finnigan) instrument with a PC1000 system controller and associated software. All samples were run on a Hypersil C18 mini-pharmaceutical column (The Nest Group) using a flow rate of 3 ml/min, an 80 s gradient of 0-99.9% CH₃CN in water/0. 1% trifluoroacetic acid/0. 1% methanol, and diode array detection. Single peaks at 224 nmol absorbance were characteristic of compounds 6 (rt=1.54 min), 7 (rt=1.54 min), and 8 (rt=1.49 min). To establish boundary conditions for detection of cleaved compounds by HPLC, standard curves were determined using pure samples of 6-8. Mock cleavage reactions (no HF present, but otherwise treated as described in the text) were carried out on resins 3-5 to determine the experimental noise for our HPLC detection method.

Robotic Implementation

Before bead arraying, 384-well plates (Genetix) were pre-wetted using a Multidrop 384 (Thermo-Labsystems) to dispense solvent. HF/py solutions were delivered using an Ivek multiplex controller module with linear actuator pump module (Ivek Corporation, http://www.ivek.comn/) coupled to an ADM-661 automatic dispensing system with TruPath 300 controller module (Creative Automation, http://www.creativedispensing.com/), and fully contained within a Captair ductless fume hood with recirculating air filtration system (Captair LabX, http:/Hwww.erlab-dfs.com/). Automated plate handling was carried out by Twister Universal microplate handlers (Zymark Corporation, http://www.zymark.com/). Evaporation of quenched reaction mixtures was done using a GeneVac HT4 Atlas evaporator with VC3000D vapor condenser (GeneVac Technologies, http://www.genevac.co.uk/). Elution of compounds from beads into 100 μl/well “mother plates” (Marsh), as well as formatting of 50 μl/well “daughter plates” (Genetix), was done with a Hydra Microdispenser 384 (Robbins Scientific Corporation, http:H/www.robsci.com/).

Small Molecule Microarrays

Small molecules were printed as described in [3], either with a microarray robot built as described by Dr. Pat O. Brown (http://cmgm.stanford.edu/pbrown/mguide/), or with an Omni-Grid1 multi-axis robot (GeneMachines, http://www.genemachines.com/). (His)6-FKBP12 was puri¢ed to homogeneity as described in [2]. Cy5-labeled protein was prepared using FluoroLink1 monofunctional reactive dye (Amersham Pharmacia Biotech) according to the manufacturer's protocol. Fluorescence detection of binding events was monitored using an ArrayWoRx biochip reader (Applied Precision, http://www.api.com/).

Cell-Based Assays

Transfer of stock solutions of 10 into assay plates (Nunc) was done using a VP386 384-pin MultiBlot1 replicator (VpP Scientific, http://www.vp-scientific.com/). Cell culture methods and the BrdU assay protocol were carried out exactly as described in [8]. Detection of assay results was carried out using X-oMAT AR film (Kodak), and multiplicative overlays of digitally scanned replicate films were prepared using Photoshop 5.0 (Adobe Systems).

### References for Example 2

[1] T. U. Mayer, T. M. Kapoor, S. J. Haggarty, R. W. King, S. L. Schreiber, T. J. Mitchison, Small molecule inhibitor of mitotic spindle bipolarity identi¢ed in a phenotype-based screen, Science 286 (1999) 971-974.

[2] G. MacBeath, A. N. Koehler, S. L. Schreiber, Printing small molecules as microarrays and detecting protein-ligand interactions en masse, J. Am. Chem. Soc. 121 (1999) 7967-7968.

[3] P. J. Hergenrother, K. M. Depew, S. L. Schreiber, Small molecule microarrays: covalent attachment and screening of alcohol-containing small molecules on glass slides, J. Am. Chem. Soc. 122 (2000) 7849-7850.

[4] A e . Furka, F. Sebestyen, M. Asgedom, G. Dibo, General method for rapid synthesis of multicomponent peptide mixtures, Int. J. Pept. Protein Res. 37 (1991) 487-493.

[5] K. S. Lam, S. E. Salmon, E. M. Hersh, V. J. Hruby, W. M. Kazmierski, R. J. Knapp, A new type of synthetic peptide library for identifying ligand-binding activity, Nature 354 (1991) 82-84.

[6] S. L. Schreiber, Target-oriented and diversity-oriented organic synthesis in drug discovery, Science 287 (2000) 1964-1969.

[7] R. B. Merrifield, Solid phase peptide synthesis: I. The synthesis of a tetrapeptide, J. Am. Chem. Soc. 85 (1963) 2149-2154.

[8] B. R. Stockwell, S. J. Haggarty, S. L. Schreiber, High-throughput screening of small molecules in miniaturized mammalian cell-based assays involving post-translational modifications, Chem. Biol. 6 (1999) 71-83.

[9] S. M. Stermson, J. B. Louca, J. C. Wong, S. L. Schreiber, Split-pool synthesis of 1,3-dioxanes leading to arrayed stock solutions of single compounds sufficient for multiple phenotypic and protein-binding assays, J. Am. Chem. Soc. 123 (2001) 1740-1747.

[10] D. S. Tan, M. A. Foley, B. R. Stockwell, M. D. Shair, S. L. Schreiber, Synthesis and preliminary evaluation of a library of polycyclic small molecules for use in chemical genetic assays, J. Am. Chem. Soc. 121 (1999) 9073-9087.

[11] D. S. Tan, M. A. Foley, M. D. Shair, S. L. Schreiber, Stereoselective synthesis of over two million compounds having structural features both reminiscent of natural products and compatible with miniaturized cell-based assays, J. Am. Chem. Soc. 120 (1998) 8565-8566.

[12] E. Bayer, Protein synthesis, Angew. Chem. Int. Ed. Engl. 30 (1991) 113-129.

[13] B. B. Brown, D. S. Wagner, H. M. Geysen, A single-bead decode strategy using electrospray ionization mass spectrometry and a new photolabile linker: 3-amino-3-(2-nitrophenyl)propionic acid, Mol. Div. 1 (1995) 4-12.

[14] J. A. Tallarico, K. M. Depew, H. E. Pelish, N. J. Westwood, C. W. Lindsley, M. D. Shair, S. L. Schreiber, M. A. Foley, An alkylsilyl-tethered, high-capacity solid support amenable to diversity-oriented synthesis for one-bead, one-stock solution chemical genetics, J. Comb. Chem. 3 (2001) 312-318.

[15] H. E. Blackwell, L. Pe\ddot{}rez, R. A. Stavenger, J. A. Tallarico, E. Cope-Eatough, S. L. Schreiber, M. A. Foley, A one-bead, one-stock solution approach to chemical genetics, part 1, Chem. Biol. 8 (2001) 1167-1182.

[16] E. J. Corey, H. Cho, C. Rucker, D. H. Hua, Studies with trialkylsilyltri£ates: new syntheses and applications, Tetrahedron Lett. 22 (1981) 3455-3458.

[17] T.-H. Chan, W.-Q. Huang, Polymer-anchored organosilyl protecting group in organic-synthesis, J. Chem. Soc. Chem. Commun. 13 (1985) 909.

[18] F. Guillier, D. Orain, M. Bradley, Linkers and cleavage strategies in solid-phase organic synthesis and combinatorial chemistry, Chem. Rev. 100 (2000) 2091-2157.

[19] T. D. Nelson, R. D. Crouch, Selective deprotection of silyl ethers, Synthesis 9 (1996) 1031-1069.

[20] H. E. Blackwell, P. A. Clemons, S. L. Schreiber, Exploiting site-site interactions on solid support to generate dimeric molecules, Org. Lett. 3 (2001) 1185-1188.

[21] Y. Hu, J. A. Porco, Alcoholysis and carbonyl hydrosilylation reactions using a polymer-supported trialkylsilane, Tetrahedron Lett. 39 (1998) 2711-2714.

[22] I. Paterson, M. Donghi, K. Gerlach, A combinatorial approach to polyketide-type libraries by iterative asymmetric aldol reactions performed on solid support, Angew. Chem. Int. Ed. Engl. 39 (2000) 3315-3319.

[23] D. C. Sherrington, Preparation, structure and morphology of polymer supports, Chem. Commun. 21 (1998) 2275-2286.

[24] D. A. Holt, J. I. Luengo, D. S. Yamashita, H.-J. Oh, A. L. Konialian, H.-K. Yen, L. W. Rozamus, M. Brandt, M. J. Bossard, M. A. Levy, D. S. Eggleston, J. Liang, L. W. Schultz, T. J. Stout, J. Clardy, Design, synthesis, and kinetic evaluation of high-a¤nity FKBP ligands and the X-ray crystal structures of their complexes with FKBP12, J. Am. Chem. Soc. 115 (1993) 9925-9938.

[25] J. F. Amara, T. Clackson, V. M. Rivera, T. Guo, T. Keenan, S. Natesan, R. Pollock, W. Yang, N. L. Courage, D. A. Holt, M. Gilman, A versatile synthetic dimerizer for the regulation of protein-protein interactions, Proc. Natl. Acad. Sci. USA 94 (1997) 10618-10623.

[26] T. Akiyama, J. Ishida, S. Nakagawa, H. Ogawara, S. Watanabe, N. Itoh, M. Shibuya, Y. Fukami, Genistein, a specific inhibitor of tyrosine-specific protein kinases, J. Biol. Chem. 262 (1987) 5592-5595.

[27] R. A. Stavenger, S. L. Schreiber, Asymmetric catalysis in diversity-oriented organic synthesis: enantioselective synthesis of 4320 encoded and spatially segregated dihydropyrancarboxamides, Angew. Chem. Int. Ed. 40 (2001) 3417-3421.

[28] H. E. Blackwell, L. Perez, S. L. Schreiber, Decoding products of diversity pathways from stock solutions derived from single polymeric macrobeads, Angew. Chem. Int. Ed. 40 (2001) 3421-3425.

Additional Information for Example 2

Loading. The copolymerized beads (500-600 μm, 1.39 meq., 800 mg, 1.11 mmol) were added to a PD-10 tube, which was capped with a septum and a plastic stopcock and flushed with Ar. After swelling with CH₂Cl₂ (10 ml), a 2.5% (v/v) solution of TMSCl in CH₂Cl₂ was added. The beads were suspended for 15 min and filtered with Ar pressure. The beads were washed with CH₂Cl2 (3×2 min) and then suspended in a solution of TfOH (10 ml, 6eq.) in CH₂Cl₂ for 15 min, during which time Ar was bubbled gently via syringe. Next, the beads were rinsed with CH₂Cl₂ (3×2 min) under Ar. The beads were suspended in CH₂Cl₂ (10 ml), freshly distilled 2,6-lutidine (1.03 ml, 8.88 mmol) and model alcohol (3.33 mmol) were successively added. The tube was capped and sealed by Teflon seal to stand for 17 h at ambient temperature. The beads were filtered and rinsed with CH₂Cl₂ (4×3 min), then dried under house vacuum.

Quenching. To determine ¹⁹F-NMR spectra of quenching simulation reactions, reagent mixing was carried out in individual wells of a 384-well microtiter plate, each containing a single bead (3), to mimic the conditions during library formatting. Spectroscopic data were tabulated based on the results of ten independent quench simulation reactions. Data were collected at 470.169MHz on a Varian AS500 spectrometer (Varian, Inc., http://www.varianinc.com/). Standards. To establish boundary conditions for detection of cleaved compounds by HPLC, standard curves were determined using pure samples of 6-8. Mock cleavage reactions (no HF present, but otherwise treated as described in the text) were carried out on resins 3-5 to determine the experimental noise for the HPLC detection method.

Manual cleavage. Manual cleavage reactions were carried out as described in the text and the Materials and Methods section. The effect of additional buffering pyridine on cleavage yields was determined by treating resins 4-5 for 30 min with 5% commercial HF/pyr. in a THF solution also containing the indicated amount of additional pyridine. Average data from nine independent reactions were converted to a percentage of the yield under “best practices” conditions (+5% pyr.).

Proteomic Assays. Slides were activated for covalent attachment of alcohols as described previously [3]. Standard microscope slides (VWR, 48300-036) were cleaned in piranha solution (70:30 v/v solution of concentrated H₂SO₄ and 30% H₂O₂) for 16 hours at room temperature. The slides were washed extensively in ddH₂O and kept in water until use. To convert to a silyl chloride surface, the slides were removed from water and dried by centrifugation. The slides were then immersed in a solution of dry THF containing 1% SOCl₂ and 0.1% DMF. The slides were incubated in this activating solution for 4 hours at room temperature. The slides were then removed, washed briefly in THF, and then placed onto the encased microarrayer platform under argon. Small molecules were printed as described previously [2,3]. Printing was carried out using a microarraying robot, constructed in this laboratory by Dr. James Hardwick and Dr. Jeff Tong according to directions provided by Dr. Pat Brown (http://cmgm.stanford.edu/pbrown/mguide/). The microarrayer typically withdraws 250 nL from a 384-well (or 96-well) plate and repetitively delivers 1 nL to defined locations on a series of activated slides. The pins were washed for 8 seconds in acetone and dried under vacuum for 8 seconds in between each sample. The arrayer was instructed to print the samples described here approximately 500 μm apart. Following printing, the slides were allowed to stand at ambient temperature for 12 hours. The slides were then washed for 2 hours in DMF, 1 hour in THF, and 1 hour in ethanol. Slides were dried by centrifugation and were at room temperature under vacuum until use. N-terminal His-tagged FKBP12 was expressed using the T5 expression plasmid pQE-30-FKBP12 (3757 bp) in M15[pREP4] (Qiagen) purified to homogeneity as described previously [3]. A starter culture was prepared by inoculating 5 mL LB medium supplemented with 100 μg/iL sodium ampicillin and 50 μg/mL kanamycin from a single colony and grown for 16 hours at 37° C. The cells were subcultured into 500 mL of the same medium at an initial OD₆₀₀ of 0.1. The culture was grown at 37° C. up to an OD₆₀₀ of 0.8. The culture was cooled to room temperature and isopropyl 1-thio-β-D-galactopyranoside (IPTG) was added to a final concentration of 1 mM. After a 16 hour induction at 30° C., the cells were harvested and frozen at −80° C. for 24 hours. The cell pellet was resuspended in 20 mL of PBS buffer supplemented with 10% (v/v) glycerol and a protease inhibitor cocktail mini-tablet (Boerhinger Mannheim). Cells were lysed by addition of 1 mg lysozyme per gram of wet cell pellet. The suspension was incubated on ice for 1 hour and followed by a 4 minute incubation at 37° C. with gentle mixing. The lysate was then kept on ice for 10 minutes. The lysate was clarified by centrifugation (28,000 g, 30 minutes, 4° C.) and loaded onto a column packed with 5 mL of Ni-NTA (Qiagen) that had been equilibrated in PBS. The column was washed with 50 mL of PBS buffer containing 10 mM imidazole. Protein bound to the column was eluted with PBS buffer containing 250 mM imidazole. The sample was dialyzed against PBS at 4° C. Cy5-labeled (His)6-FKBP12 was prepared using FluoroLink™ monofunctional reactive dye (Amersham Pharmacia Biotech) according to the manufacturer's protocol. Slides were blocked for 1 hour by incubation with PBST (PBS buffer containing 0.1% Tween-20) containing 3% BSA. After a brief rinse with PBST, fluorescently labeled protein was added a concentration of 1 μg/mL in PBST supplemented with 1% BSA. Slides were incubated with labeled protein for 30 minutes at room temperature. Slides were then washed in PBST for 3 minutes three times and dried by centrifugation. Slides were then scanned using an ArrayWoRx slide scanner (Applied Precision) at a resolution of 5 μm per pixel. The following filter sets were employed: Cy5 excitation/emission (1 second exposure) and Cy3 excitation/emission (1 second exposure).

Phenotypic Assays. The Multidrop 384 liquid dispenser (Labsystems) was used for all liquid additions, and a 24-channel wand (V&P Scientific) attached to a house vacuum source was used for all liquid aspirations. Two thousand A549 cells were seeded per well of a 384-well plate (Nalge Nunc, white, tissue culture treated) in Dulbecco's modified Eagle medium (DMEM) supplemented with 10% fetal bovine serum (FBS). Immediately upon seeding, 50 nL compound from the RAS combinatorial library was pin-transferred, one compound per well, from a 5-mM stock solution in DMSO to a final concentration of 5 μM. After 24 hours at 37° C. with 5% CO₂, 10 μL of a 10×stock of bromodeoxyuridine (BrdU) in DMEM+10% FBS was added, for a final concentration of 10 μM BrdU. The cells were incubated for 4 hours at 37° C. with 5% CO₂, cooled on ice for 15 minutes, and fixed in 50 μL 70% ethanol/30% phosphate buffered saline (PBS). All subsequent steps were performed at 4° C. Cells were washed with 90 μL cold PBS, incubated in 25 μL 2 M HCl/0.5% Tween-20 in ddH2O for 20 minutes at room temperature, and incubated instantly in 90 μL 10% 2 M NaOH/90% Hanks Buffered Salt Solution (HBSS; Gibco BRL). Cells were washed twice with 90 μL HBSS and blocked with 75 μL PBSTB (PBS, 0.1% Tween-20, 3% bovine serum albumin). Subsequently, 20 μL antibody solution, consisting of 0.5 μg/mL mouse anti-BrdU (Pharmingen), diluted 1:1000 in PBSTB, and anti-mouse IgG conjugated to horseradish peroxidase (HRP; Amersham), diluted 1:2000 in PBSTB, was added to each well. After overnight incubation at 4° C., wells were washed twice in 90 μL PBS and detected with 20 μL HRP substrate solution (ECL detection; Amersham). Film (X-OMAT AR; Kodak) was placed on top of the plates in a darkroom and developed after one to five minutes with a Kodak M35A X-OMAT processor.

### Example 3

Synthesis of Compounds Using Big Bead Technology.

This example describes an example of synthesis of a combinatorial library using a bead/linker system of the invention. See Tallarico, J., et al., J. Comb. Chem., 2001, 3, 312-318.

Currently we are using diversity-oriented split-pool synthesis to prepare structurally complex and diverse small molecules as vehicles to induce specific and novel biological phenotypes. Once a small molecule has demonstrated biological activity and the protein target identified, researchers can infer a role for that target within a biological system.ⁱ This is analogous and complimentary to methods used in classical genetics where random mutations are first generated and then screened in search of a specific cellular or physiological phenotype. This unbiased approach of using small molecules to dissect cellular circuitry is known as forward chemical genetics (FCG).ⁱⁱ

In a reverse chemical genetics (RCG) assay, which is more similar to the drug discovery process, small molecules are screened for their ability to bind a pre-selected protein target.ⁱⁱⁱ In our laboratory, small molecule printing (SMP) has provided the means for miniaturization of this process.^{iv} After identification of small molecules with suitable binding properties, experiments are performed that take advantage of their ability to modulate function rapidly and conditionally. Of course, the optimal approach to using small molecule diversity-oriented synthesis as an engine for general biological discovery is a totally integrated approach using both forward and reverse chemical genetics; this systematic approach has been given the more general name of chemical genetics.

We believe the one compound-one encoded bead strategy^{v} offers signifcant advantages as a means to generate the requisite small molecules and, consequently, large databases of novel and important protein ligands. To reduce this strategy to practice and maximize its success, it is generally preferable to be able deliver, minimally, 50 nmol of small molecule from a single synthesis bead.^{vi} This quantity of reagent allows for over one hundred FCG assays and several thousand RCG assays from a single synthesis bead with enough reagent remaining for confirmation of observed biological activity.^{vii, viii}

No solution is currently known to the problem of delivering such large quantities of small molecules on a per bead basis (typically, most large bead/linker combinations yield 5 nmol/bead).^{ix} We now report our work towards developing a novel, large (e.g., 300 mm diameter, preferably 400 mm diameter or 500 mm diameter) polystyrene bead/alkylsilyl tethered linker combination as a general solution to the problem of delivering 50 nmol of each product derived from a split-pool synthesis.^{x} This is also an integrated solution that addresses the needs of both forward and reverse chemical genetics; the released small molecules can be both pin-transferred into FCG assays and undergo SMP (recapture of the liberated —OH group) for RCG assays. Significant developments leading to this novel bead/linker combination are an improved method for functionalizing ‘naked’ large polystyrene beads, new silicon linkers for solid-phase diversity-oriented synthesis, and a modification/optimization of the solid-phase in situ Suzuki coupling conditions reported by Ellman and coworkers to synthesize diisopropylalkyl silicon functionalized resin 1 as shown in the Scheme below.^{xi}

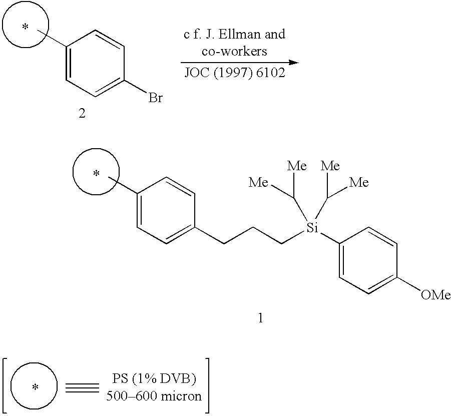

In addition to the specific examples that have been provided herein, it will be appreciated that the improved methods for for functionalizing ‘naked’ large polystyrene beads, the new silicon linkers for solid-phase diversity-oriented synthesis, and the modification/optimization of the solid-phase in situ Suzuki coupling conditions reported by Ellman and coworkers to synthesize diisopropylalkyl silicon functionalized resin 1 can also be applied more generally to other related systems.

Initial Analysis. Our initial analysis of the linker problem was primarily concerned with the end result of a diversity-oriented split-pool library synthesis; we desired a quantitative linker cleavage process that is amenable to large-scale (many beads treated simultaneously in a spatially arrayed format), does minimal damage to the final product(s), and leaves few or no impurities that are difficult to remove. Guided by similar principles used in target-oriented organic synthesis, a silicon-based linker is a preferable approach for the above as well as the following reasons^{xii}:

The attachment of starting materials is readily accomplished in high yield. Activation of silicon as a silyl chloride or silyl triflate for silyl ether synthesis is straightforward and general. For diversity-oriented synthesis, ease and efficiency of substrate loading to solid support is important, particularly with the intended goal of delivering 50 nmol of small molecule/bead.

Silicon is compatible with the widest array of modern synthetic chemistries. Because no commercially available bead/linker systems satisfied our needs, we were not encumbered with the use of known approaches that include heteroatom bond formation (typically C—O and C—N bonds) as the method of grafting the linker onto the solid support backbone.^{xiii} After removing the heteroatom(s), the linker can now be considered a bulky trialkylsilyl protecting group amenable to most modem synthetic chemistries.^{xiv} In this particular example, a strong parallel can be drawn between the chemical stability of solid support linker 1 and the known methyldiisopropylsilyl ether protecting group.^{xv}

With a diisopropylalkyl-substituted silyl ether (or other alkyl-substituted silyl ether) chosen as a flexible method of linking small molecules to the solid support, commercially available unfunctionalized 500-560 micron polystyrene macrobeads were selected to implement the approach, principally because this was the only solid support known in which each bead in a population of beads has the physical capacity to deliver 50 nmol of each small molecule.^{xvi} Of course similar approaches may be emplyed using smaller or larger beads.

Results & Discussion.

Synthesis of Bead/Linker System. Although a body of literature concerning silicon-derived solid phase linkers exists,^{xvii} to the best of our knowledge, there was no precedent for the use of large polystyrene beads (e.g., 500 micron diameter) grafted with a ‘heteroatom-free’, aliphatic linker. Inspired by the work of Woolard et al,^{xi} we chose to adapt the Suzuki coupling reaction for C—C bond formation as a route to the desired linker system. In order to carry this work forward, it was initially necessary to synthesize the Suzuki coupling partners de novo, the bromine-functionalized 1-2% dvinylbenzene (DVB) cross-linked polystyrene resin (500-560 micron) 2 and the silicon-containing alkyl borane 7 shown in Scheme 1.

(a) Bromination of polystyrene. Based on the method of Frechet,^{xviii} we subjected unfunctionalized 1-2% DVB cross-linked polystyrene, both 400-450 micron and 500-560 micron beads, to thallium acetate-catalyzed electrophilic aromatic bromination conditions in CCl₄.^{xix} Unfortunately, experimental results using this protocol were inconsistent, yielding highly colored resins with lower than expected levels of bromine incorporation.^{xx} These products were also brittle, which is disadvantageous for library synthesis.^{xxi} Increasing the amount of thallium catalyst (from 9 mol % to 18 mol % relative to bromine) and switching to CH₂Cl₂ as the reaction solvent solved these problems. Although the exact reason is unclear for this remarkable change in reactivity, one possible explanation might be increased penetration of the thallium reagent into large polystyrene beads when dissolved in CH₂Cl₂. Under these optimized conditions, the exact level of bromine incorporation was readily modulated by the amount of thallium catalyst and bromine used, always resulting in 95% yield of bromine addition with per bead levels much greater than our 50 nmol threshold limit (Example 3, Table 1). Also significant is the uniform appearance of the functionalized beads, off-white with little physical damage to their spherical geometry.^{xxii} More recently, a commercial supplier has made available large (410-500 and 500-600 micron) 1% DVB cross-linked p-bromostyrene/styrene copolymerized resins at the 1.0 and 2.0 mequiv. of Br/g loading levels which have also been used in all of the subsequent work described in this Example and the following (vide infra).^{xxiii,xxiv}

(b) Synthesis of Silicon-functionalized Alkyl Borane. With access to bromine-functionalized resin as one half of the Suzuki coupling partners, the synthesis of allyl silane 6 was readily accomplished in three steps from commercially available starting materials (Scheme 1). Diisopropylchlorosilane (3) was added to a THF solution of (4-methoxyphenyl)lithium at −78° C. and allowed to come to 23 C. overnight. Aqueous work up and filtration through silica gel produced silane 4 in 94% yield. Treatment of 4 with trichloroisocyanuric acid in CH₂Cl₂ followed by filtration under inert atmosphere provided a quantitative yield of silyl chloride 5. Allylmagnesiumbromide was then added to 5, delivering allyl silane 6 in 92% overall yield after vacuum distillation.^{xxv} The Suzuki coupling substrate, alkyl borane 7, was realized by treating a THF solution of trialkylallyl silane 6 with a nearly equimolar amount of solid 9-BBN.^{xxvi} This reagent is used without further purification or isolation.

(c) Solid-Phase Suzuki Coupling Optimization. Several reaction conditions were screened to produce the most efficient and reproducible Suzuki coupling on the 500-560 micron polystyrene beads (Example 3, Table 2). Two catalysts, Pd(PPh₃)₄ and Pd(dppf)₂Cl₂, were tested in the presence of several commonly used bases. Entry 3 describes the optimal reaction conditions. Interestingly, these conditions were identical to those originally reported by Suzuki (using NaOH as base and Pd(PPh₃)₄ as catalyst); none of the standard perturbations offered any improvement.^{xxvii,xxviii} A representative procedure is as follows (Table 3, entry 6): To a 0.17 M THF solution of alkylborane 7 (1 equiv) was added 500-600 micron 1% DVB cross-linked brominated polystyrene (0.6 equiv of resin functionalized at 2.0 mequiv of Br/g) which was allowed to fully swell in the THF/borane solution (45 min). The flask was then fitted with a condenser and Pd(PPh₃)₄ (2.5 mol %) and NaOH (2 equiv. of 2 M soln.) were added. The reaction mixture was then gently refluxed for 24 h, at which time additional palladium (2.5 mol %) was added. The reaction was then continued at reflux for a total reaction time of 40 h. Subsequent experiments proved the necessity of the additional palladium catalyst.^{xxix} The extent of silicon incorporation was confirmed by elemental analysis, typically delivering nearly quantitative yield under the optimized conditions.^{xx,xxx} Post-Suzuki coupling, the 500-600 micron beads (entry 7) possess ˜200 nmol of Si linker/bead, well above the threshold lower limit of 50 nmol/bead.

Linker Activation, Substrate Loading, and Compound Release.

(a) Silicon Activation. Because diversity-oriented synthesis strives to deliver not only structurally complex but also structurally diverse molecules in a single library, the ability to load several structurally different substrates possessing functional groups of nearly identical reactivity onto the resin under a common set of reaction conditions to a similar loading level is important to success.ⁱⁱⁱ We chose to activate the linker as trialkylsilyl triflate 8 (see Table 3) as a way to minimize the variability in loading levels between different substrates.^{xxxi,xxxii} The specific protocol was adopted directly from the work of Smith^{xxxiii} and Porco^{xxxiv}; treatment of trialkylarylsilane functionalized resin 1 with excess trifluoromethanesulfonic acid for 30 min. produced 8. Washing twice with CH₂Cl₂ removed excess acid. Complete formation of 8 was readily confirmed by MAS ²⁹Si NMR (single peak at <44.0 ppm> relative to tetramethylsilane internal standard at 0 ppm in C₆D₆). Because of the reactive nature of this species, it is recommended that the silyl triflate be used immediately after being formed.

(b) Substrate Loading. To load the alcohols shown in Table 3, the following general protocol was developed: 8 equiv. of 2,6-lutidine (relative to silyl triflate) were added to washed resin 8 followed 15 min. later by 2 equiv of the substrate alcohol dissolved in benzene.^{xxxv} This mixture is then gently agitated for 10 h, followed by thorough washing.^{xxxvi} Vacuum drying of the resin overnight delivered substrate functionalized resin 9. Significantly, even sterically demanding alcohols were loaded in good yield as witnessed by entries 3 and 4.

(c) Small Molecule Cleavage. A significant feature of this silicon linker is the ease at which it undergoes Si—O bond cleavage under the influence of a 5% solution of HF/pyridine in THF.^{xxxvii} Although this reagent is corrosive and toxic, it is a relatively mild reagent for silyl ether fluorodolysis and is dispensable by an automated liquid handler making it particularly useful for large numbers of 384-well microplates in which synthesis beads will be spatially arrayed in a one bead-one well format.^{xxxviii} We have found that 2.5 h was sufficient time for cleavage. Once complete, excess HF was quenched with methoxytrimethylsilane resulting only in volatile by-products that were readily removed under vacuum and do no harm to the released small molecules.^{xxxix} The purity of compounds cleaved by this method was very high (Table 3).^{xl}

Conclusion.

In summary, we have disclosed a route for the synthesis of an alkyl tethered diisopropylarylsilane linker on large polystyrene beads. These beads are suitable for diversity-oriented split-pool synthesis and are capable of delivering 50 nmol of small molecule per bead. Because this bead/linker system represents an enabling technologies for the practice of chemical genetics, it was important to develop a system that can be synthesized in large scale (e.g., >100 g), stored indefinitely, and used in a ‘right-off-the-shelf’ fashion. To date, our results have been very encouraging as we move forward in our efforts to bring the full power of modern organic synthesis to bear on the process of dissecting cellular circuitry. It will be noted that the methods described herein are not limited to synthesis of diisopropylarylsilane linkers but may readily be adapted to synthesis and attachment of a wide variety of alkyl tethered heteroatom free silane-based linkers.

Experimental Section.

General Methods. Starting materials and reagents were purchased from commercial suppliers and used without further purification except the following: methylene chloride (CH₂Cl₂), tetrahydrofuran (THF), and diethyl ether (Et₂O) were passed through two activated alumina columns to remove impurities prior to use (as described in Organometallics 1996, 15, 1518-1520). 2,6-Lutidine was distilled from CaH₂ under Ar atmosphere. Unfunctionalized polystyrene (400-450 and 500-560 micron 1% DVB cross-linked) was purchased from Rapp Polymere GmbH and used without further purification. Brominated polystyrene (500-600 micron 1% DVB cross-linked PS-Br, 2.0 mequiv./g and 1.0 mequiv./g) was purchased from Polymer Labs (1462-9999)¹ and used without further purification. (¹) This product is available from Polymer Labs. For pricing and availability information, they can be contacted at Polymer Laboratories Inc, Amherst Fields Research Park, 160 Old Farm Road, Amherst, Mass. 01002, USA. Tel: 413/253 9554 or 800/767 3963, Fax: 413/253 2476

Polystyrene bromination procedure (polystyrene→2)². The 500-560 μm polystyrene beads were weighed out into a 2 liter flask containing a stir bar and subsequently sealed under inert atmosphere and purged using a balloon. The 80 g of beads were then swollen in CH₂Cl₂ (1.2 L, ˜1 g of resin/15 mL of solvent) for 1 h. To this solution was added 9.4 g of thallic acetate (24.6 mmol).³ This was allowed to stir gently for 1 h.⁴ The solution turned orange with a small amount of white precipitate on the bottom of the flask. To this mixture was added 7.0 mL bromine (21.6 g/135 mmol) via syringe over a 15 minute period.⁵ After each portion of Br₂ was added, the solution would turn orange for a few seconds and then lose color. Only near the end of the addition did the color remain for longer than a few minutes. The mixture was then stirred at room temperature for 1 h at which time most of the color (but not all) had dissipated.⁶ The reaction was quenched by slow addition of 10 mL of MeOH; it was then allowed to stir for 10 min. The whole slurry was then filtered directly into a waste flask to remove solvent and dissolved catalyst. The beads on filter are then washed liberally with CH₂Cl₂. The beads were re-suspended in a second liter of CH₂Cl₂ and gently agitated for 20 minutes. This was repeated a second time. The wash procedure was completed as follows: after the two CH₂Cl₂ washes, the beads were washed with THF (1 L×20 min), THF/IPA (3:1; 1 L×20 min), THF/H₂O (3:1; 1 L×20 min.×2), DMF (1 L×20 min), THF/IPA (3:1; 1 L×20 min), THF (1 L×20 min), CH₂Cl₂ (1 L×20 min). After finishing the wash protocol, the beads were air-dried for 3 h and then placed under vacuum to remove trace solvent and water. This experiment resulted in resin that possessed 1.43 mequiv. of Br/g of functionalized resin as determined by elemental analysis. (²) For an initial reference on this procedure, see: Farrall, M. J.; Fréchet, J. M. J. J. Org. Chem. 1976, 41, 3877-3882. Early work in the Schreiber lab on this procedure began with Kris Depew. (³) Thallic acetate is very toxic therefore double gloving as a precaution is worthwhile. Also, only thallic acetate from a freshly opened bottle from Aldrich Chemical Co. should be used in the reaction—other sources as well as older batches from Aldrich have been used with limited success. (⁴) Precaution must be taken when stirring swollen beads, they are very fragile and vigorous stirring will do damage to the resin. (⁵) To ‘custom’ load brominated resin, assume that the efficiency of bromine incorporation is 95% and use the desired mmol of Br₂/g polystyrene along with the appropriate ratio of thallic acetate relative to bromine. (⁶) This procedure differs from the earlier method by not requiring heat or the use of carbon tetrachloride as solvent however, it does require the use of more thallic acetate.

Diisopropyl(4-methoxyphenyl)silane (4). A solution of p-bromoanisole (28.6 mL, 228 mmol, 1 equiv.) in THF (550 mL) was chilled to −78° C. (CO₂(s), acetone). n-BuLi (91.2 mL, 228 mmol, 2.5M in hexanes, 1 equiv.) was added via cannula over a 5 min period. After 5 min a white precipitate begins to form. The mixture was stirred for 30 min at −78° C., after which chlorodiisopropylsilane (34.6 g, 228 mmol, 1 equiv.) was slowly added via syringe. After 1 h the ice bath was removed and the solution was allowed to come to 23° C. with stirring overnight. The reaction was quenched with NH₄Cl_{(sat'd)} (50 mL) and extracted with ether (3×500 mL). The combined organic extracts were washed with brine, dried (MgSO₄), filtered and concentrated in vacuo to yield a light yellow oil. Filtration through SiO₂ (gradient: 3-5% EtOAc/hexanes) yielded 47.7 g (94%) of a colorless oil. This material could also be purified by distillation bp=76-85° C. @ 275 mTorr (40 g, 63%). TLC R_{f}=0.61 (9:1 Hexanes/EtOAc). IR (film) 2393, 1853, 1710, 1691, 1658, 1584, 1482, 1346 cm⁻¹. ¹H NMR (500 MHz, CDCl₃) δ 7.48 (d, 2H, J=8.10, C3-H, C7-H), 6.95 (d, 2H, J=8.10, C4-H, C6-H), 3.97 (s, 1H, Si—H), 3.85 (s, 3H, C1-H), 1.39 (q, 2H, J=3.0, C8-H, C11-H), 1.10 (d, 6H, J=6.5, C9-H, C10-H), 1.03 (d, 6H, J=7.5 C12-H, C13-H). ¹³C NMR (125 MHz, CDCl₃) δ 137.13, 113.73, 113.62, 55.18, 18.95, 18.72, 11.08. Anal. Calcd for C₁₃H₂₂OSi: C, 70.21; H, 9.97; Si, 12.63. Found: C, 70.43; H, 9.83; Si, 12.39.

Chloro(4-methoxyphenyl)diisopropylsilane (5). Diisopropyl(4-methoxyphenyl)silane (47.7 g, 214 mmol, 1.0 equiv.), was taken up in CH₂Cl₂ (700 mL). The solution was cooled to 0° C. and trichloroisocyanuric acid (16.6 g, 71.3 mmol, 0.33 equiv.) was carefully added in three equal portions, making sure that each portion has at least 7 min to react before the next is added (caution: adding trichloroisocyanuric acid too rapidly results in a rapid evolution of gas). The mixture was stirred at 0° C. for 40 min, followed by warming to 23° C. with stirring. The solids were filtered under an inert atmosphere and the filtrate concentrated in vacuo to yield 54.8 g (98%) of a cloudy oil. The chlorosilane, which is unstable, was used immediately and without purification in the next step.

Allyl(4-methoxyphenyl)diisopropylsilane (6). To the crude chloro(4-methoxyphenyl) diisopropylsilane (54.8 g, 214 mmol, 1.0 equiv.) was added THF (335 mL) via cannula under positive argon pressure. The solution was chilled to 0 C. and treated with allylmagnesiumchloride (128 mL, 256 mmol, 2.0 M in THF, 1.2 equiv.). After 3 h at 0° C., the solution was allowed to warm to 23 C. with stirring overnight. The mixture was treated with NH₄Cl_{sat'd} (50 mL) and the aqueous layer extracted with ether (3×500 mL). The combined organic extracts were washed with brine, dried (MgSO₄), filtered, concentrated in vacuo. The crude material was purified by silica gel chromatography (3-5% EtOAc/hexanes) to yield 52.86 g (94%) of a slightly cloudy, viscous oil. This reagent distills at 130 C. at 500 mtorr as a colorless oil. TLC R_{f}=0.40 (9:1 Hexanes/EtOAc). IR (film) 2942, 2865, 1630, 1595, 1504, 1463, 1277 cm⁻¹. ¹H NMR (500 MHz, CDCl₃) δ 7.32 (d, 2H, J=6.84, C3-H,C7-H), 6.81 (d, 2H, J=6.84, C4-H, C6-H), 5.82 (q, 1H, J=8.5, 8.5, C15-H), 4.88 (d, 1H, J=17.05, C16-Hb), 4.76 (d, 1H, J=9.77, C16-Ha), 1.82 (d, 2H, J=7.32, C14-H), 1.17 (q, 2H, J=7.3, C8-H, C11-H), 0.94 (d, 6H, J=7.3, C9-H, C10-H), 0.90 (d, 6H, J=7.3, C12-H, C13-H). ¹³C NMR (125 MHz, CDCl₃) δ 160.51, 136.48, 135.70, 125.78, 113.78, 113.62, 55.09, 19.34, 18.22, 18.17, 17.68, 11.30. Anal. Calcd for C₁₆H₂₆OSi: C, 73.22; H, 9.98; Si, 10.70.

Representative Suzuki Coupling Procedure (2→1). This procedure is included because commercially available palladium(0) did not perform as well as freshly made material. To a standard Schlenk apparatus was added palladium dichloride (275 mg, 1.55 mmol, 1.0 equiv.) and triphenylphosphine (2.04 g, 7.77 mmol, 5.0 equiv.) followed by DMSO (20.0 mL). The mixture was heated at 155 C. until total dissolution of solid material occurred. The mixture was then cooled for two minutes. Hydrazine hydrate (303 μL, 6.22 mmol, 4.0 equiv.) was added by syringe over a one minute period and the solution was immediately cooled in a cold water bath to initiate crystallization. When the first few crystals formed the flask was removed from the ice bath and covered in foil. Once formed the crystals are washed sequentially with ethanol (4×3 mL) and diethyl ether (2×1 mL). The yellow solid was protected from light and dried in vacuo overnight yielding 1.76 g of bright yellow crystals (98% yield)⁷. (⁷) Coulson, D. R. Inorg. Synth, 1972, 13, 121-124.

Alkyl borane 7 (6→7). Solid 9-BBN dimer (6.29 g, 53.0 mmol, 0.95 equiv.) was weighed out in a glove box and sealed under an argon atmosphere. Freshly distilled THF (365 mL) and allyl(4-methoxyphenyl)diisopropylsilane (6, 14.64 g, 55.8 mmol, 1.0 equiv.) were added via syringe and the mixture was allowed to stir for 3 h at 23 C. The overall concentration of the allyl(4-methoxyphenyl)diisopropylsilane in THF is 0.16 M which is the appropriate concentration for the subsequent Suzuki coupling. The yield of this reaction is assumed to be nearly quantitative.

Suzuki Coupling to produce silicon functionalized 1. To alkyl-borane containing THF solution (53.0 mmol in 365 mL of THF, 1.74 equiv.) was added the brominated polystyrene 2 (15.25 g, 2mequiv./g, 30.5 mmol of Br, 1.0 equiv.). Care was taken to maintain an argon blanket over the solution. Reagent 2 was allowed to swell for 45 min followed by addition of tetrakis(triphenylphosphine)palladium(0) (880 mg, 0.76 mmol, 0.025 equiv.) and NaOH_{aq} (61 mmol, 30.5 mL of a 2M NaOH solution, 2.0 equiv.). The reaction was then heated to mild reflux with gentle stirring for 24 h. An additional amount of Pd(0) (880 mg, 0.76 mmol, 0.025 equiv.) was added after the first 24 h and the reaction continued to reflux for another 16 h. Generally, the biphasic reaction mixture turns slightly green from its initial yellow color. Upon completion, the mixture was filtered and the beads washed repeatedly (see procedure below). Large beads (500-600 micron, in this instance) require time to take up the washing solvent. It is unnecessary to agitate the beads during the washing, but it is important to allow resin enough time to take up the solvent. Wash procedure: THF (2×200 mL×45 min), 3:1 THF/1 M aq. NaCN (1×200 mL×1 h or until all dark color is gone), 3:1 THF/H₂O (2×200 mL×45 min), 3:1 THF/IPA (2×200 mL×45 min), THF (2×200 mL×45 min), DCM (2×200 mL×45 min). The beads were air-dried overnight, then placed on a lyophilizer for 24 h, producing an almost colorless resin (white). ¹H NMR (500 MHz, CD₂Cl₂, nanoprobe) δ 7.34 (C3-H, C7-H), 6.82 (C4-H, C6-H), 5.27 (CH₂Cl₂), 3.69 (C1-H), 1.76 (C14-H), 1.48 (H₂O), 1.22(C15-H, C16-H), 1.16 (C8-H, C11-H), 0.97 (C9-H, C10-H), 0.91 (C12-H, C13-H).⁸ Anal. Found: C, 83.54; H, 8.28; Si, 4.35; Br<.02; Cl, 0.247. 2.0 mmol p-bromopolystyrene beads, quantitatively loaded with all carbon silicon linker, contain 41 mg of Si/g resin or 4.1% Si. Assuming quantitative loading, the mass of 1 g resin increases to 1.37 g, so linker loading is calculated to be ˜1.45 mequiv./mol. Thus resin loading is estimated from two elemental analyses parameters, %Si and %Br. The %Brg <0.02 by weight indicates qualitative disappearance of bromine (note that halogens can be confused by elemental analysis, therefore it is necessary to perform separate Br and Cl analysis), while percent silicon indicates the loading level. Percent silicon typically ranges from 3.79 to 4.05. The procedure used to calculate percent silicon can overestimate the actual amount of silicon by 0.2-0.3% as these numbers are calculated by weighing ash resultant from sample digestion with acid and residue combustion, which leaves some elements unresolved from silicon. 4.35% Si is equivalent to 43.5 mg Si per gram resin or 1.54 mequiv. Si/g. Actual loading used in subsequent calculations is 1.45 mequiv./g, the theoretical maximum. There are 9,350 beads/gram of 500-600 micron copolymerized p-bromopolystyrene beads with 2.0 mmol Br/g loading level. We assume quantitative conversion, justified by disappearance of bromine and appearance of appropriate amount of silicon. The number of polystyrene beads in one gram of resin is then scaled with 37% mass increase, or about 6,800 beads/g. (⁸) Linewidths, tether length, etc. effect on NMR spectra, see: P. A. Keifer, J. Org. Chem., 19996, 61, 1558-1559.

General experimental for conversion of tetralkylsilane 1 to silyl triflate functionalized resin 8. Silicon functionalized resin 1 (1.43 mequiv. Si/g) that had been dried under hi-vac for 12 h was weighed (200 mg, 0.286 mmol, 1 equiv.) into a 10 mL polypropylene PD-10 column fitted with a teflon™ stopcock and swollen in CH₂Cl₂ (2.0 mL, 10 mL of solvent/g of resin) under N₂ atmosphere for 30 min. The solvent was then drained under positive N₂ pressure and 3.8 mL of a 4% tifluoromethanesulfonic acid/CH₂Cl₂ solution (6 equiv. of TfOH relative to Si) was added by syringe. The resin turned bright red/orange upon acid treatment and was then gently agitated for 30 min. while still under N₂ atmosphere. Once activation was completed, two CH₂Cl₂ washes removed excess acid.

Loading of trans-2-phenyl-1-cyclohexanol onto resin 8. Treatment of 8 with 2,6-lutidine (0.27 mL, 8 equiv. relative to Si) for 15 min. followed by addition of 0.5 mL of an azeotropically dried 1.0 M solution of trans-2-phenyl-1-cyclohexanol (2 equiv) resulted in a colorless resin. The beads are then gently agitated for an additional 10 h under N₂ atmosphere. The beads were drained, exposed to atmosphere, and subjected to the following wash protocol: CH₂Cl₂ (2×3 mL×45 min.), THF (2×3 mL×30 min.), THF/IPA (3:1, 2×3 mL×30 min.), THF/H₂O (3:1, 2×3 mL×30 min.); THF/IPA (3:1, 2×3 mL×30 min.), DMF (2×3 mL×30 min.), THF (2×3 mL×30 min.). The resin was air-dried for 3 h and then placed under hi-vac for 24 h to remove trace solvent and H₂O. The mass of the loaded and dried resin was 207.0 mg, indicating an apparent loading efficiency of 74% based on weight gain. Single bead cleavage experiments resulted in an average per bead loading of 137 nmol/bead (69% efficiency, data included in table). See the supporting information for details. This procedure is applicable for loading all alcohols listed in Table 3.

MAS-¹H NMR of trans-2-phenyl-1-cyclohexanol (500 MHz, CD₂Cl₂, nanoprobe) δ 7.30-7.20 (5 H), 3.72-3.64 (broad s, 1 H), 2.45-2.40 (broad, 1 H), 2.13-2.10 (broad, 1H), 1.89-1.85 (broad, 1H), 1.79-1.75 (broad, 1H), 1.60-1.32 (complex, 6 H).

Cleavage of 8 from resin. Vacuum-dried resin 8 was weighed (100.0 mg) out into a solvent-resistant scintillation vial and allowed to swell in 1.0 mL of THF for 30 min. The THF solution was removed and replaced with a fresh 0.95 mL of THF and 0.05 mL of HF/pyridine solution (7:3 ratio of HF/pyr, available from Aldrich Chemical Co.). The vial was sealed and agitated for 3 h at which time 0.1 mL of methoxytrimethylsilane was added to quench unreacted HF. (Note: quench is mildly exothermic therefore use caution). The beads are further agitated for 30 min to ensure complete quenching. The solution was removed and the beads washed twice with additional 1.0 mL portions of fresh THF. All solvents were combined and concentrated in vacuo, delivering 16.3 mg of trans-2-phenyl-1-cyclohexanol as a white solid. Based on the assumption that 100% of the material loaded onto the resin is cleaved and recovered, this amount of material represents 71% of the theoretical maximum or approximately 143 nmol/bead.

Supporting Information Available. Experimental details regarding equipment, disposables and representative single bead cleavage experiments and analyses are available (7 pages) free of charge via the Internet at http://pubs.acs.org and are additionally presented below.

TABLE 1

Bromination of Polystyrene

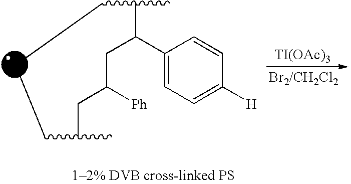

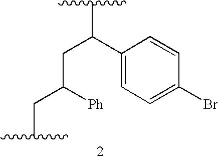

| | Resin Size | Br Loading | | |
|---|---|---|---|---|
| Entry | (microns) | (theoretical^{a}) | nmol/bead | % yield |
| (1) | 500-560 | 0.97 mequiv/g | 92 | |
| | | (1.00) | | |
| (2) | 500-560 | 1.31 mequiv/g | 127 | 96 |
| | | (1.36) | | |
| (3)^{b} | 500-560 | 1.43 mequiv/g | 147 | 96 |
| | | (1.49) | | |
| (4)^{c} | 500-560 | 1.74 mequiv/g | 176 | 95 |
| | | (1.84) | | |
| (5) | 400-450 | 1.86 mequiv/g | 95 | 96 |
| | | (1.93) | | |

^{a}Determined by elemental analysis (ref. xx).
^{b}Performed on > 80 g scale.
^{c}This loading is identical on a per bead basis to commercially available 500-600 micron beads (after factoring in size distribution) at 2 mequiv of Br/g (ref xxiv).

TABLE 2
Solid phase Suzuki Coupling Optimization.^{a}

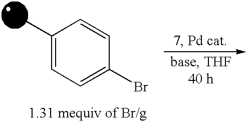

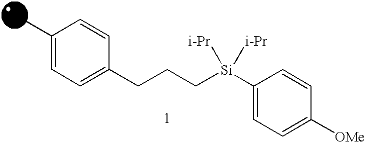

| | | Pd(PPh₃)₄ | Pd(dppf)₂ |
|---|---|---|---|
| Entry | Base | yield, (Si linker/bead) | yield, (Si linker/bead) |
| (1) | Na₂CO₃ | 88%, (112 nmol) | 77%, (98 nmol) |
| (2) | K₂CO₃ | 91%, (116 nmol) | 46%, (58 nmol) |
| (3) | NaOH | 98%, (124 nmol) | 80%, (102 nmol) |
| (4) | NaOMe | 83%, (105 nmol) | 76%, (97 nmol) |
| (5) | K₃PO₄ | 98%, (124 nmol) | 51%, (65 nmol) |
| (6)^{b} | NaOH | 98%, (204 nmol) | — |

^{a}Entries 1-5 were run on 500-560 micron PS beads with 127 nmol of Br/bead initial loading.
^{b}Entry 6 represents the commercially available 500-600 micron beads initially loaded at 208 mequiv of Br/g as determined by elemental analysis.

TABLE 3
Small Molecule Loadings.

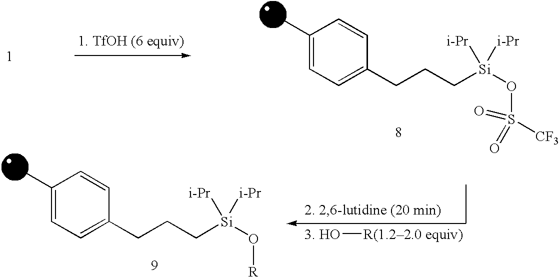

| Entry | Substrate (equiv used) | | Recovered (per bead)^{a} |
|---|---|---|---|
| (1)^{b} | 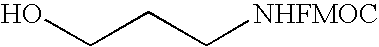 | (1.5) | (90% (114 nmol) |
| (2)^{c} | 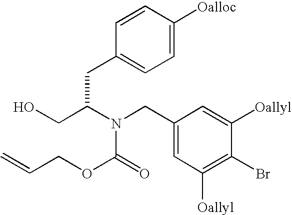 | (1.2) | (73% (146 nmol) |
| (3) | 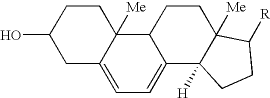 | (2.0) | (76% (152 nmol) |
| | 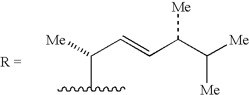 | | |
| (4)^{d} | trans-2-phenyl-1-cyclohexanol | (2.0) | 69% |
| | | | (137 nmol) |
| (5)^{d} | 4-bromo-3, 5-dimethylphenol | (2.0) | 79% |
| | | | (158 nmol) |

^{a}Material recovered is an indirect determination of the efficiency of substrate loading. All material recovered was > 95% pure as determined by HPLC analysis using a photodiode array detector.
^{b}Entry 1 was run on 500-560 micron beads and loading was determined by standard Fmoc cleavage analysis.
^{c}Loading was run on ˜ 6 gof resin in a single experiment.
^{d}These loadings were confirmed by HPLC analysis and recovered material.

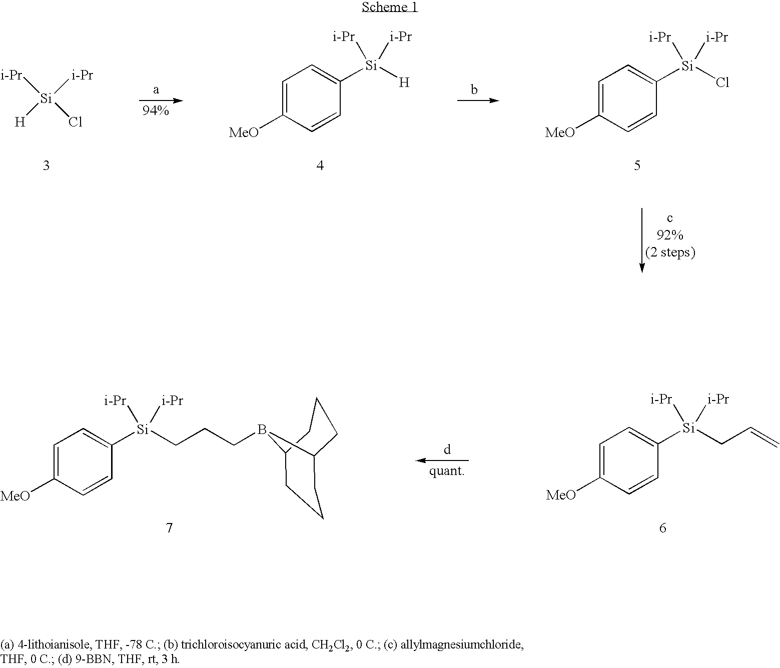

Additional Information for Example 3

I. General Methods:

Starting materials and reagents were purchased from commercial suppliers and used without further purification except the following: methylene chloride (CH₂Cl₂), tetrahydrofuran (THF), and diethyl ether (Et₂O) were passed through two activated alumina columns to remove impurities prior to use (as described in Organometallics 1996, 15, 1518-1520). 2,6-Lutidine was distilled from CaH₂ under Ar atmosphere. Unfunctionalized polystyrene (400-450 and 500-560 micron 1% DVB cross-linked) was purchased from Rapp Polymere GmbH and used without further purification. Brominated polystyrene (500-600 micron 1% DVB cross-linked PS-Br, 2.0 mequiv./g and 1.0 mequiv./g) was purchased from Polymer Labs (1462-9999)⁹ and used without further purification. (⁹) This product is available from Polymer Labs. For pricing and availability information, they can be contacted at Polymer Laboratories Inc, Amberst Field Research Park, 160 Old Farm Road, Amherst, Mass. 01002, USA. Tel: 413/253 9554 or 800/767 3963, Fax: 413/253 2476

Oxygen- or moisture-sensitive solution-phase reactions were carried out under N₂ or Ar atmosphere in oven-dried (140 C., 4 h) glassware. Small-scale solid phase reactions (1-50 mg resin) were performed in 2.0 mL fritted polypropylene Bio-Spin® chromatography columns (Bio-Rad Laboratories, Hercules, Calif.; 732-6008). Medium-scale solid phase reactions (50-250 mg) were performed in 10.0 mL polypropylene PD-10 columns (Pharmacia Biotech, Piscataway, N.J.; 17-0435-01). Agitation of solid phase reactions was accomplished using a 360 rotator from Bamstead/Thermolyne (model no. 415110). Large-scale solid phase reactions (>500 mg) were carried out in oven-dried (140 C., 4 h), fritted peptide synthesis vessels available from ChemGlass (Vineland, N.J.) equipped with inlets for vacuum and inert atmosphere. Small scale linker cleavage reactions (1 bead→50 mg of resin) were carried out in 1.5 mL eppendorf tubes ( ). Larger scale cleavage reactions are performed in Wheaton scintillation vials available from Fisher Scientific (cat no. 03-341-25A).

Air- and/or moisture-sensitive liquids were transferred by syringe or cannula and were introduced into the reaction vessel through rubber septa or through a stopcock under N₂ or Ar positive pressure. Air- and/or moisture-sensitive solids were transferred in a glove box or atmosbag. Unless otherwise stated, reactions were stirred with a teflon™ covered stir bar and carried out at RT. Concentration refers to the removal of solvent using a Büichi rotary evaporator followed by use of a vacuum pump at approximately 1 torr. Column chromatography was performed using Merck 60 Å (230-400 mesh ASTM) silica gel. Thin layer chromatography (TLC) analyses were performed using Merck 60 F₂₅₄ 0.25 μm silica gel plates. Vacuum removal of solvents for linker cleavage reactions was accomplished using a Genevac HT-4 Atlas Evaporator.

Proton nuclear magnetic resonance spectra (¹H NMR) were measured on a Varian Unity Inova 500 spectrometer (500 MHz). Carbon nuclear magnetic resonance (¹³C NMR) spectra were recorded on a Varian Unity Inova 500 spectrometer (125 MHz). Magic Angle Spinning NMR (MAS NMR) were recorded on a Varian Unity Inova 500 spectrometer using a NanoProbe. (¹) (a) Fitch, W. L.; Detre, G.; Holmes, C. P.; Shoolery, J. N.; Keifer, P. A. J. Org. Chem. 1994, 59, 7955-7956. (b) Keifer, P. A.; Baltusis, L.; Rice, D. M.; Tymiak, A. A.; Shoolery, J. N. J. Magn. Reson., Series A 1996 119, 65-75. All ¹H and ¹³C NMR chemical shifts are reported in ppm downfield from tetramethylsilane (0.00 ppm) or residual solvents (CDCl₃, 7.26 ppm/77.7 ppm). Infrared spectra (IR) were measured on a Bruker Vector 22 ATR-based Infrared Spectrometer, νₘₐₓ in cm⁻¹.

Elemental analyses (Anal) were performed by Robertson Microlit Laboratories Inc., Madison, N.J. 07940 and were reported in percent atomic abundance. Tandem high pressure liquid chromatography/mass spectral (LCMS) analyses were performed on a Micromass Platform II mass spectrometer in atmospheric pressure chemical ionization (APCI) mode after separation performed on a Waters Alliance 2690 separations module. The actual separation was performed on a Waters Symmetry® C₁₈ 3.5 μm, 2.1×50 mm column with a flow rate of 0.4 mL/min and a 12 min gradient of 15-100% CH₃CN in H₂O, constant 0.1% formic acid buffer using a Waters 996 photodiode array detector.

II. Representative Substrate Loading Procedure.

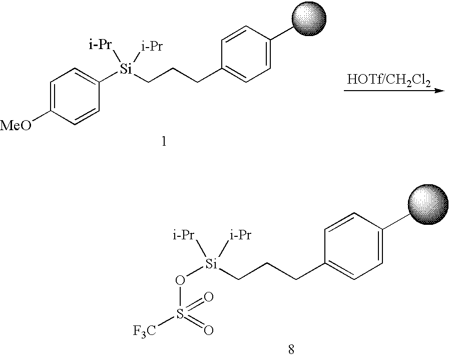

Conversion of tetralkylsilane to silyl triflate functionalized resin 8. Silicon functionalized resin 1 (1.43 mequiv. Si/g) that had been dried under hi-vac for 12 h was weighed (200 mg, 0.286 mmol, 1 equiv.) into a 10 mL polypropylene PD-10 column fitted with a teflon™ stopcock and swollen in CH₂Cl₂ (2.0 mL, 10 mL of solvent/g of resin) under N₂ atmosphere for 30 min. The solvent was then drained under positive N₂ pressure and 3.8 mL of a 4% tifluoromethanesulfonic acid/CH₂Cl₂ solution (6 equiv. of TfOH relative to Si) was added by syringe. The resin turned bright red/orange upon acid treatment and was then gently agitated for 30 min. while still under N₂ atmosphere. Once activation was completed, two CH₂Cl₂ washes removed excess acid.

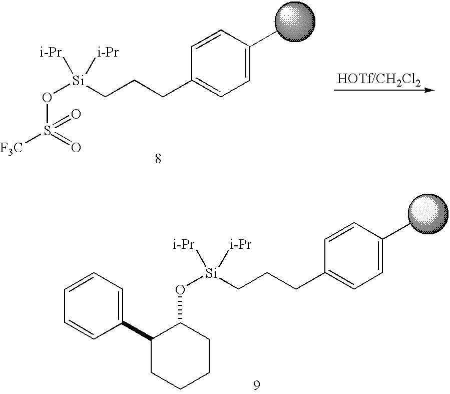

Loading of trans-2-phenyl-1-cyclohexanol onto resin 8. Treatment of 8 with 2,6-lutidine (0.27 mL, 8 equiv. relative to Si) for 15 min. followed by addition of 0.5 mL of an azeotropically dried 1.0 M solution of trans-2-phenyl-1-cyclohexanol (2 equiv) resulted in a colorless resin 9. The beads are then gently agitated for an additional 10 h under N₂ atmosphere. The beads were drained, exposed to atmosphere, and subjected to the following wash protocol: CH₂Cl₂ (2×3 mL×45 min.), THF (2×3 mL×30 min.), THF/IPA (3:1, 2×3 mL×30 min.), THF/H₂O (3:1, 2×3 mL×30 min.); THF/IPA (3:1, 2×3 mL×30 min.), DMF (2×3 mL×30 min.), THF (2×3 mL×30 min.). The resin was air-dried for 3 h and then placed under hi-vac for 24 h to remove trace solvent and H₂O. The mass of the loaded and dried resin was 207.0 mg, indicating an apparent loading efficiency of 74% based on weight gain.

MAS-¹H NMR, 5 beads, (500 MHz, CD₂Cl₂, nanoprobe) δ 7.34 (C3-H, C7-H), 6.82 (C4-H, C6-H), 5.27 (CH₂Cl₂), 3.69 (C1-H), 1.76 (C14-H), 1.48 (H₂O), 1.22(C15-H, C16-H), 1.16 (C8-H, C11-H), 0.97 (C9-H, C10-H), 0.91 (C12-H, C13-H)

III. General Cleavage Process. Bulk cleavage of resin for determination of loading by compound recovery.

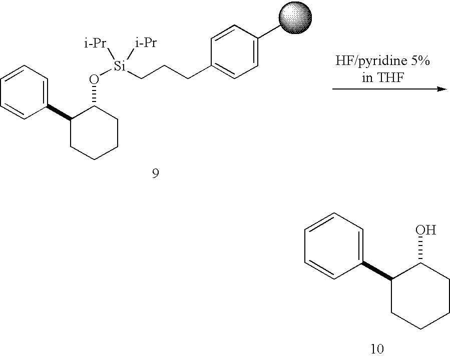

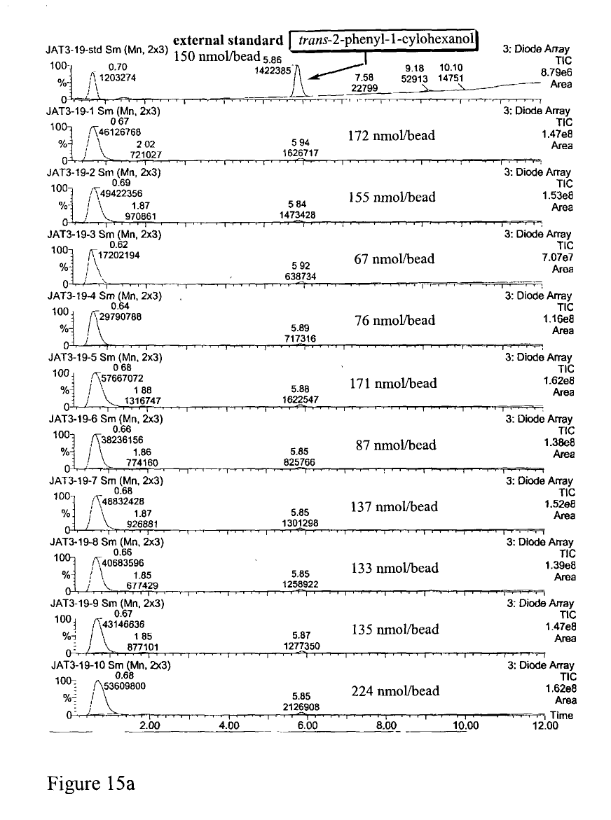

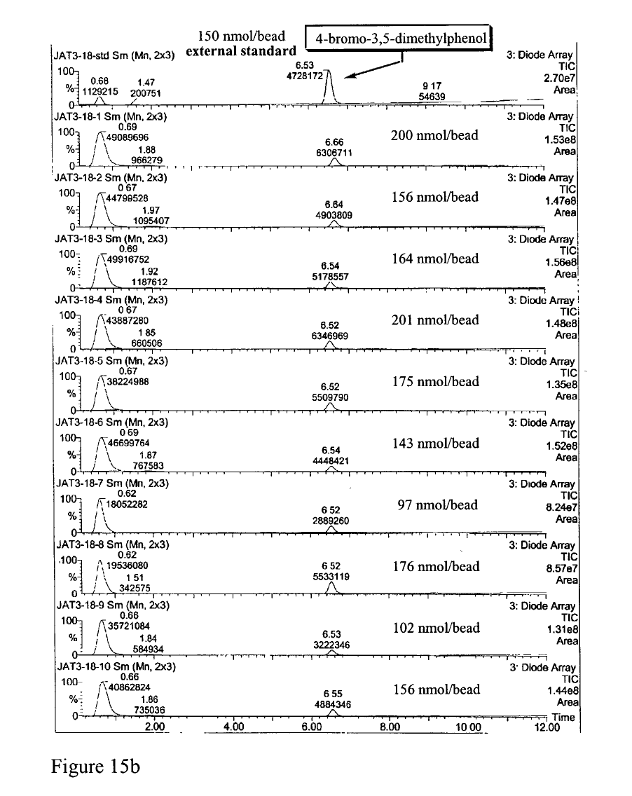

Vacuum-dried resin 9 was weighed (100.0 mg) out into a solvent-resistant scintillation vial and allowed to swell in 1.0 mL of THF for 30 min. The THF solution was removed and replaced with a fresh 0.95 mL of THF and 0.05 mL of HF/pyridine solution (7:3 ratio of HF/pyr, available from Aldrich Chemical Co.). The vial was sealed and agitated for 3 h at which time 0.1 mL of methoxytrimethylsilane was added to quench unreacted HF. (Note: quench is mildly exothermic therefore use caution). The beads are further agitated for 30 min to ensure complete quenching. The solution was removed and the beads washed twice with additional 1.0 mL portions of fresh THF. All solvents were combined and concentrated in vacuo, delivering 16.3 mg of trans-2-phenyl-1-cyclohexanol as a white solid. Based on the assumption that 100% of the material loaded onto the resin is cleaved and recovered, this amount of material represents 71% of the theoretical maximum or approximately 143 nmol/bead. p General determination of average per bead loading by HPLC analysis for entries 4 and 5, Table 3. Ten separate beads from the above loading experiment (loading of trans-2-phenyl-1-cyclohexanol, vide supra) were placed into 10 unique 500 μL Eppendorf tubes and swelled in 47.5 μL of THF for 30 min. To each of these tubes was added 2.5 μL of a HF/pyridine solution. Each of the tubes were sealed and agitated for 2.5 h followed by addition of 10 μL of TMSOMe. The tubes were resealed and agitated for an additional 30 min. to ensure complete quenching of HF. The remaining solvent was removed from the Eppendorf tubes using an HT-4 Atlas Evaporator from Genevac (˜30 min) and the remaining residue was taken up in 100 μL of THF to separate the cleaved molecules from the bead. The beads were washed with a second 50 μL of THF for 30 min. and was pooled with the original wash solution. All THF was removed under reduced pressure, the residue taken up in 100 μL of CH₃CN and used in a 5 μL injection onto the LCMS system (details described in the General Methods section) for LC analysis relative to an external standard. This same experiment was performed on resin loaded with 4-bromo-3,5-dimethylphenol (Table 3, entry 5). Data for both experiments (LC/traces) is included in FIG. 15.

### References for Example 3

(ⁱ) Schreiber, S. L. Bioorg. Med. Chem. 1998, 6, 1127-1152. (b) Mitchison, T. J. Chem. Biol. 1994, 1, 3-6. (c) http://iccb.med.harvard.edu. (d) http://www-schreiber.chem.harvard.edu.

(ⁱⁱ) Mayer, T. U.; Kapoor, T. M.; Haggarty, S. J.; King, R. W.; Schreiber, S. L.; Mitchison T. J., Science 1999, 286, 971-974.

(ⁱⁱⁱ) (a) Peterson, R. T.; Link, B. A.; Dowling, J. E.; Schreiber, S. L. Proc. Natl. Acad. Sci. U.S.A. 2000, 97(24), 12965-9. (b) Tan, D. S.; Foley, M. A.; Stockwell, B. R. Shair, M. D.; Schreiber, S. L. J. Am. Chem. Soc. 1999, 121, 9073-9087.

(^{iv}) Schreiber, S. L. Science 2000, 287, 1964-1969.

(^{v}) (a) MacBeath, G.; Koehler, A. N.; Schreiber, S. L. J. Am. Chem. Soc. 1999, 121, 7967-7968. (b) Hergenrother, P. J.; Depew, K. M.; Schreiber, S. L. J. Am. Chem. Soc. 2000, 122, 7849-7850.

(^{vi}) Currently we use a modification of the binary encoding method first described by Still to elucidate the chemical history of each synthesis bead. Ohlmeyer, M. H.; Swanson, R. N.; Dillard, L. W.; Reader, J. C.; Asouline, G.; Kobayashi, R.; Still, W. C. Proc. Natl. Acad. Sci. 1993, 90, 10922-10926. Modifications of this technique will be disclosed in the near future.

(^{vii}) The 50 nmol of small molecule will give rise to a 5 mM stock solution in 10 μL of DMSO. Our current infrastructure readily supports the automated handling of these volumes of reagents and solvents for library arraying requirements and biological assays.

(^{viii}) Using robotic pin-transfer methods, our largest pin transfer array consumes approximately 100 nL of stock DMSO solution upon each use. For the process of compound printing (see ref. iv), approximately one nanoliter of stock solution is removed from each well in a typical source plate.

(^{ix}) This amount of material also allows for the use of traditional methods of analysis (MAS-NMR, HPLC, LC-MS, etc.) to assist in determining the identity of each small molecule in the event that the binary encoding/decoding protocol fails. See ref v.

(^{x}) For an example of a high capacity bead using heteroatom grafted dendrimers, see: Fromont, C.; Bradley, M. J. Chem. Soc., Chem. Commun. 2000, 283-284.

(^{xi}) (a) Furka, A.; Sebestyen, F.; Asgdom, M.; Dibo, G. Int. J. Pept. Protein Res. 1991, 37, 487-493. (b) Lam, K. S.; Salmon, S. E.; Hersh, E. M.; Hruby, V. J.; Kazmierski, W. M.; Knapp, R. J. Nature 1991, 354, 82-84.

(^{xii}) Woolard, F. X.; Paetsch, J.; Ellman, J. A. J. Org. Chem. 1997, 62, 6102-6103.

(^{xiii}) For review of the relative stabilities and cleavage conditions of silylethers, see: Nelson, T. D.; Crouch, R. D. Synthesis 1996, 1031-1069.

(^{xiv}) We felt it was important to exclude heteroatoms from the tether (graft unit) to remove the possibility of unexpected reactivity/interference that could erode confidence in each split-pool synthetic step. For a recent review of linker chemistry, see: Guillier, F.; Orain, D.; Bradley, M. Chem. Rev. 2000,100, 2091-2157. See also: James, I. W. Tetrahedron 1999, 55, 4855-4946.

(^{xv}) Greene, T.; Wuts, P. G. M. Protecting Groups in Organic Synthesis; 3^{rd} ed.; Wiley: New York, 1999.

(^{xvi}) Kunz, H.; Waldman, H. Comprehensive Organic Synthesis; Trost, B. M.; Fleming, I., Eds.; Pergammon: Oxford, 1991; Vol. 6, p. 631.

(^{xvii}) This product is available from RAPP Polymere GmbH. Unfunctionalized 1-2% DVB cross-linked polystyrene 500-560 micron beads number 11,500 beads/g; unfunctionalized 400-450 micron beads number 23,000 beads/g.

(^{xvii}) (a) Stover, H. D. H.; Lu, P. Z.; Frechet, J. M. J. Polymer Bulletin 1991, 25, 575-582. (b) Boehm, T. L.; Showalter, H. D. H. J. Org. Chem. 1996, 61, 6498-6499. (c) Stranix, B. R.; Liu, H. Q.; Darling, G. D. J. Org. Chem. 1997, 62, 6183-6186. (d) Hu, Y.; Porco, J. A.; Labadie, J. W.; Gooding, O. W. J. Org. Chem. 1998, 63, 4518-4521. (e) Plunkett, M. J.; Ellman, J. A. J. Org. Chem. 1997, 62, 2885-2893. (f) Brichn, C. A.; Kirshbaum, T.; Bäuerle, P. J. Org. Chem. 2000, 65, 352-359.

(^{xix}) (a) Stover, H. D. H.; Lu, P. Z.; Frechet, J. M. J. Polymer Bulletin 1991, 25, 575-582. (b) Boehm, T. L.; Showalter, H. D. H. J. Org. Chem. 1996, 61, 6498-6499. (c) Stranix, B. R.; Liu, H. Q.; Darling, G. D. J. Org. Chem. 1997, 62, 6183-6186. (d) Hu, Y.; Porco, J. A.; Labadie, J. W.; Gooding, O. W. J. Org. Chem. 1998, 63, 4518-4521. (e) Plunkett, M. J.; Ellman, J. A. J. Org. Chem. 1997, 62, 2885-2893. (f) Briehn, C. A.; Kirshbaum, T.; Bäuerle, P. J. Org. Chem. 2000, 65, 352-359.

(^{xix}) Farrall, M. J.; Frechet, J. M. J. J. Org. Chem. 1976, 41, 3877-3882.

(^{xx}) McKillop, A.; Bromley, D.; Taylor, E. C. Tetrahedron Lett. 1969, 10, 1623-1626.

(^{xxi}) Bromine and silicon loading levels were determined by elemental analyses of the resin beads. All analyses were performed by Robertson Microlit Labs, Madison, N.J., 07940.

(^{xxii}) Good mechanical stability of the large polystyrene beads is critical. During the course of a library synthesis, the repeated handling, chemical reactions, swelling/solvation, and drying steps associated with multiple synthetic transformations tends to degrade the overall physical integrity of the resin.

(^{xxiii}) This information comes from visual inspection of the resin using a dissecting microscope. (xxiv) Polymer Laboratories Inc., Amherst, Mass. 01002, USA, has provided these products.

(^{xxv}) The calculated per bead loading of the 1.0 mequiv. of Br/g (10,240 beads/g) and 2.0 mequiv. of Br/g (9,400 beads/g) 500-600 micron PS beads are 98 nmol and 212 nmol respectively.

(^{xxvi}) This sequence of reactions has been successfully done on a 300 g scale.

(^{xxvii}) Brown, H. C. Organic Synthesis via Boranes; Wiley: New York, 1975.

(^{xxviii}) Miyaura, N.; Suzuki, A. Chem. Rev. 1995, 95, 2457-2483.

(^{xxix}) For previous examples of solid phase Suzuki coupling procedures, see: Larhed, M.; Lindeberg, G.; Hallberg A. Tetrahedron Lett. 1996, 37, 8219-8222. (b) Piettre, S. R.; Baltzer, S. Tetrahedron Lett. 1997, 38, 1197-1200. (c) Vanier, C.; Wagner, A.; Mioskowski, C. Tetrahedron Lett. 1999, 40, 4335-4338.

(xxx) Time course and catalyst loading levels were examined; data not shown.

(xxxi) This chemistry has been performed on >100 g of resin in a single experiment.

(^{xxxii}) Corey, E. J.; Cho, H.; Rücker, C.; Hua, D. H. Tetrahedron Lett. 1981, 22, 3455-3458.

(^{xxxiii}) This silicon linker has also been activated and loaded as the diisopropylalkylsilyl chloride through treatment with a CH₂Cl₂ solution of dry HCl.

(^{xxxiv}) Smith, E. M. Tetrahedron Lett. 1999, 40, 3285-3288.

(^{xxxv}) Porco, J. A.; Hu, Y. Tetrahedron Lett. 1999, 40, 3289-3292.

(xxxvi) Typically the substrate alcohol is dissolved in benzene and trace water is removed azeotropically using a rotary evaporator. Methylene chloride can also be used for dissolution of the substrate when solubility in benzene is problematic.

(^{xxxvii}) The volume of a solvent used in each washing step is approximately 10 mL/g of resin. See ref. xxxviii for details about developing wash protocols.

(^{xxxviii}) This reagent is approximately a 7:3 mixture of HF and pyridine. This reagent can be further buffered with additional pyridine in the cleavage cocktail.

(^{xxxix}) Blackwell, H. E. et al, unpublished results.

(xl) Hu, Y.; Porco, J. A. Tetrahedron Lett. 1998, 39, 2711-2714.

(^{xli}) For examples of specific chemistries performed on this bead/linker combination, see: (a) Lee, D.; Sello, J.; Schreiber, S. L. Organic Letters 2000, 2, 709-712. (b) Spring, D. R.; Krishnan, S.; Schreiber S. L. J. Am. Chem. Soc. 2000, 122, 5656-5657. (c) Blackwell, H. E.; Clemons, P. A.; Schreiber, S. L. Presented at the 220th National Meeting of the American Chemical Society, Washington, D.C., August 2000.

### Example 4

Decoding Products from Stock Solutions Derived from Single Polymeric Macrobeads

Methods, Description, and Results

As discussed above, efficient phenotypic and proteomic screening of small molecules derived from diversity-oriented organic syntheses [1] is facilitated by a library realization platform [2] that 1) produces a sufficient quantity of compound per bead to perform many hundreds of assays [3] and 2) supports reliable compound structure identification. To facilitate the latter, solid-phase library-encoding strategies [4] have been developed that allow the identity of the compounds to be inferred postsynthesis directly from individual beads.[5] We applied a chemical encoding strategy [6] to a high-capacity (1.4 mequivg⁻¹˜100 nmol/bead), 500±600 mm polystyrene (PS) solid support (FIG. 2), [3] an important element of the “one-bead, one-stock-solution” technology platform described above and in [7]. We have now discovered that the stock solutions of compounds cleaved from individual beads contain sufficient tags to allow the structures of their corresponding small molecules to be inferred reliably. Two methods used for the decoding compound libraries such as the library of 4320 dihydropyrancarboxamides reported in the Stavenger, R., et al., J. Angew. Chem. Int. Ed., 2001, 40(18) are described [8] The encoding method features structurally related chloroaromatic diazoketone “tags”, [6b] which are introduced through an acylcarbene insertion into the phenyl rings of PS catalyzed by [Rh2(O2CCPh3)4] (1) to yield cycloheptatrienes (FIG. 2) [9]. To decode a library compound, the tags are cleaved oxidatively from the solid support with ceric ammonium nitrate (CAN) to yield free alcohols, [10] which are then silylated (with N,O-bis-(trimethylsilyl)acetimide, BSA) and injected directly onto a gas chromatograph equipped with electron-capture detection (GC/ECD) for analysis (each tag trimethylsilyl ether has a unique GC retention time). For lowloaded solid support (˜100 pmol/bead) it has been postulated that the carbene inserts predominantly into the support due to the greater proportion of the support relative to compound [11, 12]. However, because our PS macrobead supports contain considerably more bound compound than standard solid-phase resin (that is, ˜50±100 nmol/bead) [3] and we used higher relative tag concentrations to encode the supports effectively,[11] we decided to investigate the amount of tag attached directly to the support-bound synthetic compounds. On one hand, we were concerned that acylcarbene insertion could cause significant compound contamination. However, as described below, we have found that this does not appear to be the case. In addition, we have found that the level of compound tagging is sufficient to allow effective decoding without complicating analyses of the properties of the compounds (<0.1%).

During our optimization of the encoding/decoding procedure on PS macrobeads, we observed that the tag peaks in the GC traces for beads decoded before compound cleavage were dramatically stronger than those for beads decoded after compound cleavage.[7] Treatment of the solid support with hydrogen fluoride/pyridine (HF+py) to cleave the attached compounds from the silyl ether linker (FIG. 2) did not concurrently cleave the attached tags; [13] therefore, we surmised that the difference in relative peak area could be explained by significant incorporation of the diazoketone tags into the support-bound compound. These compound-associated tags were being cleaved simultaneously with those inserted into the PS macrobead and were thus inflating the relative tag peak intensities.

To verify tag insertion into the support-bound compound during the encoding process, PS macrobeads were loaded with a model compound, N-(5-hydroxypentyl)-4-methylbenzamide to form 10 (FIG. 3) [14]. A portion of 10 (20 mg) was treated with HF+py to effect cleavage of the model compound and to generate an unadulterated compound sample, before any encoding took place. Another 20 mg sample was encoded with four chloroaromatic diazoketone tags following our optimized procedure [7a] and subsequently treated with HF+py to release the attached compound. The two compound samples were characterized by ¹H NMR spectroscopy and liquid chromatography/mass spectrometry (LC-MS), and both techniques failed to show any detectable tag incorporation into the compound as described further below. However, these results did not preclude direct compound decoding by GC/ECD because the level of sensitivity of the EC detector (subpicomolar) is far greater than that of 1H NMR spectroscopy and LC-MS detection techniques.

The solid compound obtained from a single, encoded bead was subjected to the identical decoding conditions developed for bead decoding, and GC/ECD analysis of the silylated product revealed that the tags had inserted into the compound.[15] Furthermore, the signals obtained when the compound itself was decoded were dramatically stronger (˜10² times) than those detected from bead decoding performed after compound cleavage. Indeed, 20 pmol of each tag were observed when the entire compound stock solution obtained from one macrobead was subjected to the optimized decoding protocol. While not wishing to be bound by any theory, the reason for this apparent increase in tag amount relative to standard bead decoding can be attributed, in part, to the higher efficiency of the homogeneous oxidative cleavage reaction in solution, relative to the heterogeneous reaction on polymeric support [16]. This higher efficiency observed in stock-solution decoding enables decoding times and temperatures to be reduced to 2 h and 25° C., respectively, to obtain similar tag amount measurements. The amount of tag inserted into the compound per bead is approximately 2-3 orders of magnitude less than the amount used for encoding. We estimate that the stock solution derived from a single bead (20 μL of a ˜5 mm solution per bead) encoded at this level remains analytically pure (>99.9%) and anticipate that the trace impurities introduced by tag insertion should not compromise a one-bead, one-compound approach.

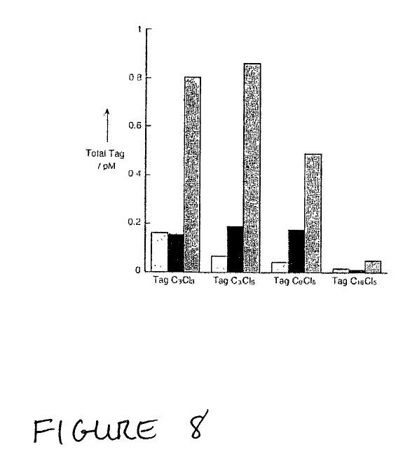

The ability to decode directly from a compound's stock solution should be advantageous in many circumstances, including those where GC signals from bead decoding are not sufficiently strong or clear to assign a chemical structure to a “hit” or if the parent bead cannot be located (which may occur in solid phase synthesis, for example, due to static electricity). As direct stock-solution decoding gives GC signals approximately 100 times greater than those obtained from bead alone, we postulated it should be possible to decode a compound, or “hit”, by decoding only a small portion of its respective stock solution. To this end, fractions (that is, 1%, 5%, 10%, 50%, and 100%) of a stock solution of the model compound of 12 in FIG. 3 cleaved from a single encoded bead were subjected to the decoding procedure. This analysis demonstrated that submitting just 1% of the compound's stock solution was sufficient for clear and reproducible decoding, equivalent or superior to bead-decoding data (See FIG. 8).

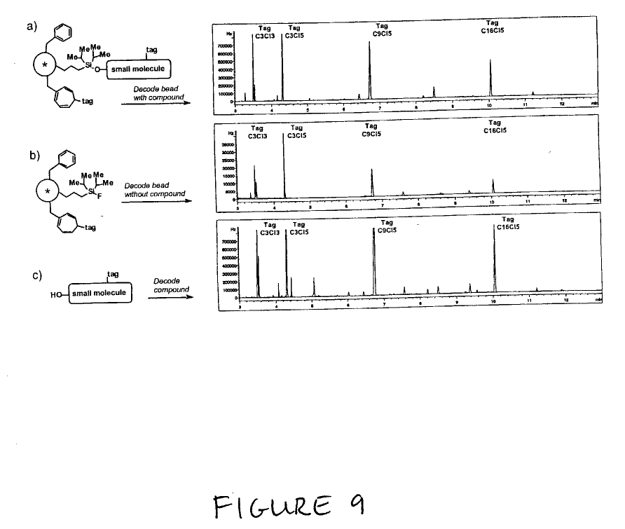

As shown in FIG. 9, we now have three ways to determine the chemical history of a bead relying solely on encoding: the bead can be decoded either 1) before compound cleavage, 2) after compound cleavage, or 3) from a fraction of its respective compound's stock solution. To the best of our knowledge, the latter is the first example of decoding chemical tags covalently attached to compounds as a strategy for structure elucidation after splitpool synthesis [11].

In order to further demonstrate the reliability of stock-solution decoding to determine the chemical history of an actual library member, this method was applied to selected samples from the dihydropyrancarboxamide library described Stavenger, et al., referenced above. The synthesis described therein resulted in 54 (27×2 enantiomers) separate portions of PS support containing 4320 stereochemically and structurally distinct compounds. Two beads were taken from each of the 54 final library pools, arrayed into individual tubes, and treated with HF+py to release the compounds from the beads. Samples from each of the stock solutions for the 108 compounds (diluted to 5 μm in acetonitrile) were subjected to MS (atmospheric pressure chemical ionization, APCI) analysis, and the corresponding beads and/or aliquots of stock solution were subjected to GC/ECD decoding to compare the two results and check for agreement between mass spectral and chemical encoding data.

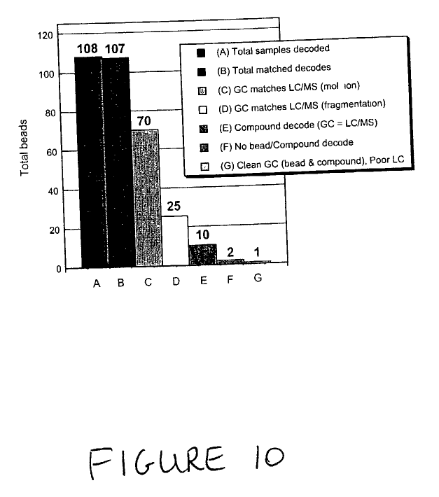

We were gratified to observe that, for 107 of the 108 samples, the structure assignments made by GC bead and/or stock-solution decoding were in complete agreement with the MS data. The majority of the samples could be decoded from their respective beads; however, the bead-decoding GC traces for 10 of the samples were not easily interpreted, so a fraction (˜5%) of the corresponding stock solutions was subjected to direct decoding as described above. All 10 of these samples yielded clean GC traces that decoded for a compound identical to that assigned by MS, which thus demonstrates the feasibility of direct stock-solution decoding with real library compounds. Finally, there were two cases in which the parent bead was lost, so it was necessary to resort to stock solution decoding. Once more, stock-solution decoding agreed with the MS data for these missing beads. Of note, there was only one example of the 108 samples analyzed where the compound identity assigned by GC decoding could not be confirmed by MS: In this case, both bead and stock solution GC decoding were identical but neither a parent molecular ion nor a compound fragment was observed in the MS spectrum which was in agreement with this structure assignment [17]. FIG. 10 shows a graph of the observed complementarity between the GC and MS decoding of 108 samples from the dihydropyrancarboxamide library. Table 1 shows binary decoding data from GC and LC/MS analysis of 108 beads from the dihydropyrancarboxamide library. (BB=building block). Underlined bead numbers correspond to compound decoded samples. GC and MS data for bead #105 could not be correlated. Representative GC, LC, and MS traces and chemical structure assignments, which demonstrate bead and stock-solution decoding, respectively, are shown in FIG. 2 of Blackwell, H., et al., Agnew. Chem. Int. Ed. 2001, 40(18), 3421-3425 for samples 12 and 48 [18].

The successful partial decoding of the dihydropyrancarboxamide library validates not only our encoding/decoding protocol, but also the use of direct stock-solution decoding as a reliable decoding strategy. In addition to the partial decoding of 2.5% of the theoretical total library members for this study, further stock solution decoding has been done on an ad hoc basis during the biological screening of this library (data not shown), and to date all of the 25 hits have been successfully identified. Currently, the “hits” that were difficult to decode previously from a split-pool library on PS macrobeads that used a completely different set of reactions and substrates are being decoded directly from the stock solutions with the compound decoding approach reported herein. Preliminary results of four different diversity-oriented synthetic pathways suggest stock-solution decoding will be feasible with many different chemistries. Obtaining the chemical history of small molecules synthesized through diversity-oriented syntheses by direct stock-solution decoding should expedite compound structure elucidation, minimize storage requirements (parent beads can be discarded), and be amenable to automation with a laboratory liquid-handling robot. This approach, therefore significantly facilitates the “global” decoding of entire split-pool libraries [19].

### References for Example 4 Methods, Description, and Results Section

[1] a) S. L. Schreiber, Science 2000, 287, 1964-1969; b) S. L. Schreiber, Bioorg. Med. Chem. 1998, 6, 1127-1152.

[2] For an additional report of our technology platform, see: S. M. Stemson, J. B. Louca, J. C. Wong, S. L. Schreiber, J. Am. Chem. Soc. 2001, 123, 1740-1747.

[3] J. A. Tallarico, K. M. Depew, H. E. Pelish, N. J. Westwood, C. W., Lindsley, M. D. Shair, S. L. Schreiber, M. A. Foley, J. Comb. Chem. 2001, 3, 312-318.

[4] For a review of encoding methods, see: A. W. Czarnik, Curr. Opin. Chem. Biol. 1997, 1, 60-66.

[5] For an overview of encoded combinatorial library synthesis, see: D. S. Tan, J. J. Burbaum, Curr. Opin. Drug Discovery Dev. 2000, 3, 439-453.

[6] a) M. H. J. Ohlmeyer, R. N. Swanson, L. W. Dillard, J. C. Reader, G., Asouline, R. Kobayashi, M. Wigler, W. C. Still, Proc. Natl. Acad. Sci. USA 1993, 90, 10922-10 926; b) H. P. Nestler, P. A. Bartlett, W. C., Still, J. Org. Chem. 1994, 59, 4723-4724.

[7] a) H. E. Blackwell, L. Perez, R. A. Stavenger, J. A. Tallarico, E. Cope-Eatough, M. A. Foley, S. L. Schreiber, Chem.& Biol., 2001, 8, 1167-1182; b) P. A. Clemons, A. N. Koehler, B. K. Wagner, T. G. Sprigings, D. R. Spring, R. W. King, S. L. Schreiber, M. A. Foley, Chem. & Biol., 2001, 8, 1183-1195.

[8] R. A. Stavenger, S. L. Schreiber, Angew. Chem. 2001, 113, 3525±3529; Angew. Chem. Int. Ed. 2001, 40, 3417-3421.

[9] M. A. McKervey, D. N. Russell, M. F. Twohig, J. Chem. Soc. Chem. Commun. 1985, 491-492.

[10] R. Johansson, B. Samuelsson, J.Chem. Soc. Perkin Trans. 1 1984, 2371-2374.

[11] Still and co-workers reported tag insertion into a support-bound tetrapeptide during the encoding process, in a ratio of 1:8 with tag inserted into the support (PS bead diameter ˜50-100 mm, loading ˜100 pmolg⁻¹). The authors used substantially less diazoketone tag (1 pmol tag/bead) to encode their synthesis resin than we found to be sufficient for 500-600 mm PS macrobeads (20 nmol tag/bead), presumably due to a more efficient heterogeneous insertion reaction on smaller beads. See refs. [6b] and [7].

[12] For a review of RhII-catalyzed carbene reactions, see: J. Adams, D. M. Spero, Tetrahedron 1991, 47, 1765-1808.

[13] Direct silylation and GC analysis of compounds cleaved from beads with HF+py without CAN treatment indicated only negligible amounts of tag silyl ethers, which ruled out the compound cleavage step as a major tag-cleavage event.

[14] Prepared by an EDC-mediated coupling between 5-amino-1-pentanol and p-tolylacetic acid in CH2Cl2 in the presence of triethylamine and 4-dimethylaminopyridine (DMAP). See: J. C. Sheehan, P. A. Cruickshank, G. L. Boshart, J. Org. Chem. 1961, 26, 2525-2528.

[15] Attempts to determine the structure of the tag-inserted compound have been unsuccessful so far. However, we postulate that an aromatic-ring and/or N—H insertion reaction occurred. See ref. [12].

[16] Given the different efficiencies of the homogeneous and heterogeneous tag-cleavage reactions, we could not determine quantitatively if more tag was inserting into the compound rather than the bead, or vice versa.

[17] This discrepancy can be attributed to either unsuccessful chemical reaction in one or more of the library steps and/or poor ionization by MS, and serves as a reminder that encoding records the sequence of chemical reactions to which a bead is subjected, not the structure of the compound.

Additional Information for Example 4

Synthetic Methods

General Methods. Reagents were obtained from Aldrich Chemical Co. and used without further purification. Standard reaction solvents (CH₂Cl₂, toluene, THF, Et₂O, and DMF) were obtained from J. T. Baker (HPLC grade) and purified by passage through two solvent columns prior to use. The CH2Cl2 and toluene purification systems are composed of one activated alumina (A-2) column and one supported copper redox catalyst (Q-5 reactant) column. The THF and Et₂O purification systems are composed of two activated alumina (A-2) columns, and the DMF purification system is composed of two activated molecular sieve columns. See: Pangbom, A. B.; Giardello, M. A.; Grubbs, R. H.; Rosen, R. K.; Timmers, F. J. Organometallics 1996, 15, 1518-1520. Brominated polystyrene beads (Br—PS; bead diameter=500-600 μm; 2 mequiv/g) were obtained from Polymer Labs, Inc. (Amherst, Mass., 01002, USA.) and derivatized with the silyl ether linker according to the procedure described in Example 3. Diazoketone chloroaromatic tags were purchased from Pharmacopeia, Inc. (Box 5350, Princeton, N.J. 08543-5350, USA). Rh₂(O2CC(Ph)₃)₄ was purchased from TCI (Japan). N,O-bis (trimethylsilyl)acetimide (BSA) was purchased in 1 mL sealed glass ampoules from Pierce Chemical Co. and used immediately after opening. N-(5-hydroxy-pentyl)-4-methyl-benzamide was prepared via a PyBOP mediated coupling between 5-amino-1-pentanol and p-tolylacetic acid in DMF (Coste, J.; Lenguyen, D.; Castro, B. Tetrahedron Lett. 1990, 31, 205-208).

Solid Phase Reactions. Small-scale solid-phase reactions (5-10 mg resin) were performed in 500 μL polypropylene Eppendorf tubes with mixing provided by a Vortex Genie-2 vortexer fitted with a 60 microtube insert. Larger scale solid-phase reactions (20-500 mg resin) were performed in 2 mL fritted polypropylene Bio-Spin® chromatography columns (Bio-Rad) or 10 mL fritted polypropylene PD-10 columns (Pharmacia Biotech) with 360° rotation on a Bamstead-Thermolyne Labquake™ Shaker. After small-scale reactions, resin samples were transferred to 2 mL BioSpin® columns. Resin samples in polypropylene columns were washed with organic solvents on a Vac-Man®Laboratory Vacuum Manifold (Promega) fitted with nylon 3-way stopcocks (Bio-Rad). The following standard wash procedure was used: 3×THF, 3×DMF, 3×THF, 3×CH₂Cl₂.

Compound cleavage reactions. Resin samples were transferred via spatula to 500 μL Eppendorf tubes and suspended in Ar-degassed HPLC grade THF followed by pyridine and hydrogen fluoride-pyridine (Aldrich, HF(70%)/pyridine(30%)) in a ratio of 90:5:5. Samples were then sealed with Parafilm and gently agitated on a vortexer for 30 min. Methoxytrimethylsilane (TMSOMe) was added, and the samples were sealed with Parafilm and placed on a vortexer for an additional 30 min. The supernatant fluid was removed, transferred to another Eppendorf tube, and concentrated in vacuo.

Purification and Analysis. HPLC was performed on a Nest Group (Southborough, Mass.) Hypersil C18 100 Å 3 μM, 4.6 mm×6 cm column using a flow rate of 3 mL/min and a 4 min gradient of 0-99.9% CH₃CN in H₂O/0.1% TFA, constant 0.1% MeOH with diode array UV detection. NMR spectra were recorded on Varian Inova 500 MHz and 400 MHz instruments. Chemical shifts are expressed in ppm relative to TMS (0.00 ppm) or residual solvents. LC/MS data was obtained on a Micromass Platform LCZ mass spectrometer in atmospheric pressure chemical ionization (APCI) mode attached to a Waters 2690 HPLC system. LC/MS chromatography was performed on a Waters Symmetry C18 3.5 μM, 2.1 mm×50 mm column using a flow rate of 0.4 mL/min and a 10 min gradient of 15-100% CH₃CN in H₂O, constant 0.1% formic acid with 200-450 nmol detection on Waters 996 photodiode array detector. GC/ECD data was obtained on a Hewlett Packard 6890 Gas Chromatograph fitted with a 7683 series injector and autosampler, split-splitless inlet, μ-ECD detector, and a J&W DB1 15 m×0.25 mm×0.25 μm column. (Gradient start temperature: 110° C.; hold 1 min, ramp 45° C./min to 250° C., hold 2 min, ramp 15° C./min to 325° C., hold 2 min. Flow rate: constant flow, 1 mL/min. Inlet is purged at 1 min with flow rate 60 mL/min, reduced to 20 mL/min at 2 min).

Encoding and Decoding Protocols

Representative Bead Encoding Procedure. Place 20 dry beads (approximately 3 mg resin) in a 700 μL Eppendorf polypropylene tube. Prepare a fresh 8.4 mM (in each tag) solution in dry CH₂Cl₂ in an oven-dried, Teflon capped glass vial. (NOTE: The tag concentration can be cut by one-half to one-fifth, and the tags will still be readable by GC (the late tags will be weak). This might be necessary for large library syntheses where a large quantity of tag is required, or if more than 4 tags are used in each tagging step. Use the same volume of tag solution as described below.) Add 50 μL of the tag solution to the Eppendorf tube. Set the tube to shake for 45 min at room temperature on a tabletop orbital shaker. Prepare a 4.4 mg/mL solution of the catalyst, rhodium triphenylacetate (Rh₂(O₂CC(Ph)₃)₄) in dry CH₂Cl₂ under Ar in an oven-dried, Teflon capped glass vial. (NOTE: The catalyst concentration can be reduced by one-half to one-fifth and the tags will still be readable by GC (the late tags will be weak). Use the same volume of catalyst solution as described below.) Add 50 μL of the catalyst solution to the resin and keep the Eppendorf in agitation for 16 h (overnight) at room temperature. Wash the resin in a 1 mL BioRad tube 2×15 min CH₂Cl₂, 16 h (overnight) THF, 2×15 min THF, and 2×15 min CH₂C₂. Dry the resin under house vacuum for ca. 15 min before proceeding to compound cleavage. Compound Cleavage: Place the beads into a 700 μL Eppendorf tube. Add 100 μL of freshly prepared 5% (HF/py)/THF solution (v/v). Set the tube to shake for 90 min at room temperature on a tabletop Eppendorf shaker. Quench HF by adding 200 μL TMSOMe to the tube. Set the tube to shake for 30 min at room temperature on a tabletop Eppendorf shaker. Collect the filtrate (if desired), and wash the resin: 3×5 min CH₂Cl₂, 3×5 min THF, and 3×5 min CH₂Cl₂. under house vacuum for at least 1 h before decoding.

Representative Bead or Compound Decoding Procedure. For bead decoding, place one bead into a 100 μL autosampler glass sample insert (Waters) with the aid of tweezers. For compound decoding, add an aliquot of the compound stock solution (from an individual bead) to the autosampler glass insert and remove all of the solvent by applying a steady stream of N₂ to the opening of the insert. A 0.24 M solution of CAN in 5:1 THF/H₂O is prepared (132 mg CAN/0.83 mL dry, degassed THF+0.17 mL doubly-distilled H₂O) in an oven-dried vial. This solution should be prepared immediately before use. Add 5 μL of the CAN solution to the glass autosampler insert. Add 8 μL of dry decane to the glass insert and then centrifuge the insert in a Micro-Centrifuge to separate the two layers. Place the insert in a 2 mL glass autosampler vial (VWR) and cap tightly. Seal with Parafilm, and heat the vial at 37° C. for 21 h (in a standard laboratory incubator). Allow the sample to cool to room temperature, and remove the glass insert from the autosampler vial. Sonicate the insert for 1-10 min. Centrifuge the insert again in the Micro-Centrifuge. Use a 200 μL Pipetman equipped with a gel-loading tip to remove the top decane layer and transfer it to a new 100 μL autosampler glass insert. (After heating overnight, the CAN layer will be colorless, so caution must be used to not contaminate the decane layer with CAN in transfer.) Prepare a 1:1 BSA/decane solution in an oven-dried vial. This solution should be prepared immediately before use. Add 1.0 μL of this BSA solution to the decane layer in the glass insert. Spin down the insert in the Microfuge for 30-40 sec to ensure efficient mixing of the BSA solution with the sample. Place the insert in a 2 mL GC autosampler vial (Hewlett Packard), cap tightly, and store at 0° C. until GC analysis.

TABLE 1
Binary decoding data from GC and LC/MS analysis of 108 beads from the
dihydropyrancarboxamide library. (BB = building block). Underlined bead numbers correspond
to compound decoded samples. GC and MS data for bead #105 could not be correlated.

| Bead | Tag | Tag | Tag | Tag | Tag | Tag | Tag | Tag | Tag | | | | Expected | |
|---|---|---|---|---|---|---|---|---|---|---|---|---|---|---|
| # | 2B | 4B | 1A | 2A | 3A | 4A | 5A | 6A | 7A | BB1 | BB2 | BB3 | mass | Observed mass |
| 1 | 0 | 1 | 1 | 0 | 1 | 1 | 0 | 0 | 0 | H | F | A | 572 | M + H |
| 2 | 1 | 0 | 0 | 1 | 0 | 1 | 1 | 0 | 0 | G | J | A | 539 | M + H |
| 3 | 0 | 1 | 1 | 0 | 1 | 1 | 0 | 0 | 0 | H | F | B | 552 | M + H |
| 4 | 1 | 1 | 0 | 0 | 0 | 0 | 0 | 1 | 0 | B | D | B | 435 | M + H |
| 5 | 1 | 0 | 0 | 1 | 1 | 1 | 0 | 0 | 0 | G | F | C | 447 | M + H |
| 6 | 0 | 0 | 0 | 1 | 1 | 0 | 1 | 0 | 0 | D | G | C | 447 | M + H |
| 7 | 1 | 1 | 0 | 0 | 1 | 0 | 0 | 0 | 0 | E | A | D | 441 | M + H |
| 8 | 1 | 0 | 0 | 1 | 1 | 0 | 0 | 0 | 0 | G | A | D | 401 | M + H |
| 9 | 0 | 0 | 0 | 1 | 0 | 0 | 0 | 1 | 0 | D | D | E | 514 | M + H |
| 10 | 0 | 1 | 0 | 0 | 0 | 0 | 0 | 0 | 1 | C | E | E | 500 | M + H |
| 11 | 1 | 0 | 0 | 1 | 1 | 0 | 1 | 0 | 0 | G | G | F | 473 | M + H |
| 12 | 1 | 1 | 0 | 0 | 1 | 1 | 0 | 0 | 0 | E | F | F | 479 | M + H |
| 13 | 1 | 1 | 0 | 0 | 1 | 0 | 1 | 0 | 0 | E | G | G | 592 | M + H |
| 14 | 1 | 1 | 0 | 0 | 0 | 0 | 0 | 0 | 1 | E | E | G | 596 | M + H |
| 15 | 0 | 0 | 0 | 1 | 0 | 1 | 1 | 0 | 0 | D | J | H | 540 | M − EtOH |
| 16 | 1 | 0 | 0 | 0 | 1 | 0 | 0 | 1 | 0 | A | H | H | 497 | M + H |
| 17 | 0 | 1 | 0 | 0 | 1 | 1 | 0 | 0 | 0 | B | F | I | 582 | M + H |
| 18 | 0 | 0 | 0 | 1 | 1 | 0 | 1 | 0 | 0 | D | G | I | 614 | M − BBI_D |
| 19 | 0 | 1 | 0 | 0 | 0 | 1 | 1 | 0 | 0 | B | J | J | 640 | M + H |
| 20 | 0 | 0 | 1 | 0 | 0 | 1 | 1 | 0 | 0 | C | J | J | 638 | M + H |
| 21 | 1 | 0 | 0 | 0 | 0 | 0 | 0 | 1 | 0 | A | D | K | 563 | M + H |
| 22 | 0 | 0 | 1 | 0 | 1 | 0 | 1 | 0 | 0 | C | G | K | 559 | M − EtOH |
| 23 | 1 | 0 | 0 | 0 | 1 | 1 | 0 | 0 | 0 | A | F | L | 497 | M + H |
| 24 | 0 | 1 | 0 | 0 | 0 | 1 | 1 | 0 | 0 | B | J | L | 617 | M + H |
| 25 | 0 | 1 | 1 | 0 | 0 | 0 | 1 | 0 | 0 | H | C | M | 694 | M + H |
| 26 | 0 | 0 | 0 | 1 | 0 | 1 | 1 | 0 | 0 | D | J | M | 545 | M + H |
| 27 | 1 | 0 | 0 | 1 | 0 | 1 | 1 | 0 | 0 | G | J | N | 540 | M + H |
| 28 | 0 | 1 | 1 | 0 | 0 | 0 | 1 | 0 | 0 | H | C | N | 655 | M + H |
| 29 | 1 | 0 | 0 | 0 | 0 | 1 | 0 | 0 | 0 | A | B | O | 383 | M + H |
| 30 | 0 | 0 | 0 | 1 | 0 | 0 | 1 | 0 | 0 | D | C | O | 485 | M − BBI_D |
| 31 | 0 | 1 | 0 | 0 | 0 | 1 | 1 | 0 | 0 | B | J | P | 607 | M + MeOH—H₂O |
| 32 | 1 | 1 | 0 | 0 | 1 | 0 | 0 | 0 | 1 | E | I | P | 637 | M + MeOH—H₂O |
| 33 | 1 | 0 | 0 | 1 | 1 | 0 | 0 | 1 | 0 | G | H | Q | 644 | M + H |
| 34 | 1 | 0 | 1 | 0 | 1 | 0 | 0 | 1 | 0 | F | H | Q | 684 | M + H |
| 35 | 1 | 0 | 0 | 1 | 1 | 0 | 0 | 0 | 0 | G | A | R | 413 | M + H |
| 36 | 0 | 0 | 0 | 1 | 1 | 1 | 0 | 0 | 0 | D | F | R | 419 | M − EtOH |
| 37 | 0 | 1 | 1 | 0 | 0 | 1 | 1 | 0 | 0 | H | J | S | 1049 | M + H |
| 38 | 0 | 1 | 1 | 0 | 1 | 1 | 0 | 0 | 0 | H | F | S | 945 | M + H |
| 39 | 0 | 0 | 1 | 0 | 1 | 1 | 0 | 0 | 0 | C | F | T | 513 | M + H |
| 40 | 0 | 0 | 1 | 0 | 0 | 1 | 1 | 0 | 0 | C | J | T | 617 | M + H |
| 41 | 0 | 0 | 0 | 1 | 1 | 0 | 0 | 0 | 1 | D | I | U | 469 | M − EtOH |
| 42 | 1 | 0 | 1 | 0 | 1 | 0 | 1 | 0 | 0 | F | G | U | 515 | M − C₄O₂H₅ |
| 43 | 0 | 0 | 0 | 1 | 0 | 0 | 1 | 0 | 0 | D | C | V | 558 | M − EtOH |
| 44 | 0 | 1 | 0 | 0 | 0 | 0 | 1 | 0 | 0 | B | C | V | 560 | M + H |
| 45 | 1 | 0 | 1 | 0 | 1 | 0 | 0 | 0 | 1 | F | I | W | 505 | M − C₄O₂H₅ |
| 46 | 1 | 0 | 0 | 1 | 0 | 1 | 1 | 0 | 0 | G | J | W | 507 | M + H |
| 47 | 0 | 0 | 1 | 0 | 1 | 0 | 0 | 0 | 1 | C | I | X | 576 | M − EtOH |
| 48 | 1 | 0 | 1 | 0 | 0 | 0 | 0 | 0 | 1 | F | E | X | 626 | M + H |
| 49 | 1 | 0 | 0 | 0 | 1 | 0 | 0 | 0 | 1 | A | I | Y | 413 | M + H |
| 50 | 0 | 0 | 1 | 0 | 0 | 1 | 1 | 0 | 0 | C | J | Y | 469 | M + H |
| 51 | 1 | 0 | 0 | 1 | 0 | 0 | 1 | 0 | 1 | G | D | A | 487 | M + H |
| 52 | 0 | 0 | 0 | 1 | 1 | 0 | 1 | 1 | 0 | D | I | A | 463 | M − EtOH |
| 53 | 1 | 0 | 0 | 1 | 0 | 1 | 0 | 1 | 0 | G | A | B | 375 | M − H₂O |
| 54 | 0 | 1 | 1 | 0 | 0 | 1 | 0 | 1 | 0 | H | A | B | 512 | M + H |
| 55 | 0 | 0 | 1 | 0 | 0 | 1 | 0 | 1 | 0 | H | A | C | 544 | M − MeOH |
| 56 | 0 | 0 | 0 | 1 | 1 | 1 | 0 | 0 | 1 | D | H | C | 488 | M + H |
| 57 | 0 | 0 | 1 | 0 | 1 | 1 | 0 | 1 | 0 | F | G | D | 515 | M − EtOH |
| 58 | 1 | 0 | 0 | 0 | 0 | 0 | 1 | 0 | 1 | A | D | D | 445 | M + H |
| 59 | 0 | 0 | 0 | 1 | 0 | 1 | 0 | 0 | 1 | D | B | E | 456 | M + H |
| 60 | 1 | 0 | 0 | 1 | 0 | 1 | 0 | 0 | 1 | G | B | E | 490 | M + H |
| 61 | 1 | 0 | 0 | 1 | 0 | 1 | 0 | 0 | 1 | G | B | F | 433 | M + H |
| 62 | 0 | 1 | 1 | 0 | 0 | 0 | 0 | 1 | 1 | H | E | F | 614 | M + H |
| 63 | 1 | 0 | 0 | 0 | 1 | 0 | 1 | 0 | 1 | A | J | G | 574 | M + H |
| 64 | 0 | 0 | 0 | 1 | 1 | 1 | 0 | 0 | 1 | D | H | G | 559 | M + H |
| 65 | 0 | 0 | 0 | 1 | 1 | 0 | 1 | 1 | 0 | D | I | H | 498 | M − EtOH |
| 66 | 0 | 0 | 1 | 0 | 1 | 0 | 1 | 0 | 1 | C | J | H | 540 | M + H |
| 67 | 0 | 1 | 0 | 0 | 0 | 1 | 0 | 0 | 1 | B | B | I | 576 | M + H |
| 68 | 0 | 1 | 1 | 0 | 0 | 0 | 0 | 1 | 1 | D | E | I | 618 | M + H |
| 69 | 1 | 0 | 0 | 1 | 1 | 1 | 0 | 1 | 0 | G | G | J | 602 | M + H |
| 70 | 1 | 0 | 0 | 1 | 0 | 1 | 0 | 1 | 0 | G | A | J | 528 | M + H |
| 71 | 1 | 1 | 0 | 0 | 0 | 0 | 1 | 0 | 1 | E | D | K | 651 | M + H |
| 72 | 1 | 0 | 1 | 0 | 0 | 0 | 0 | 1 | 1 | F | E | K | 637 | M + H |
| 73 | 1 | 0 | 1 | 0 | 1 | 1 | 1 | 0 | 0 | F | F | L | 585 | M + H |
| 74 | 1 | 0 | 0 | 1 | 0 | 0 | 0 | 1 | 1 | G | E | L | 583 | M + H |
| 75 | 1 | 0 | 1 | 0 | 1 | 1 | 0 | 0 | 1 | F | H | M | 590 | M + H |
| 76 | 1 | 0 | 1 | 0 | 0 | 0 | 0 | 1 | 1 | F | E | M | 553 | M − C₄O₂H₅ |
| 77 | 0 | 0 | 1 | 0 | 1 | 0 | 1 | 0 | 1 | C | J | N | 506 | M + H |
| 78 | 1 | 0 | 0 | 1 | 0 | 0 | 1 | 1 | 0 | G | C | N | 518 | M + H |
| 79 | 1 | 0 | 0 | 0 | 0 | 0 | 1 | 1 | 0 | E | C | O | 559 | M + H |
| 80 | 1 | 1 | 0 | 0 | 1 | 1 | 1 | 0 | 0 | E | F | O | 477 | M + H |
| 81 | 1 | 0 | 0 | 1 | 1 | 1 | 0 | 1 | 0 | G | G | P | 569 | M + MeOH—H₂O |
| 82 | 0 | 1 | 1 | 0 | 0 | 0 | 1 | 1 | 0 | H | C | P | 754 | M + H |
| 83 | 0 | 0 | 0 | 1 | 1 | 1 | 0 | 1 | 0 | D | G | Q | 569 | M − EtOH |
| 84 | 1 | 0 | 0 | 1 | 1 | 1 | 0 | 1 | 0 | G | G | Q | 603 | M − H₂O |
| 85 | 1 | 0 | 0 | 0 | 1 | 0 | 1 | 1 | 0 | A | I | R | 467 | M + H |
| 86 | 0 | 0 | 1 | 0 | 0 | 0 | 0 | 1 | 1 | C | E | R | 457 | M − EtOH |
| 87 | 0 | 1 | 0 | 0 | 1 | 1 | 0 | 1 | 0 | B | G | S | 810 | M + H |
| 88 | 1 | 0 | 0 | 0 | 1 | 1 | 0 | 1 | 0 | A | G | S | 794 | M + H |
| 89 | 1 | 0 | 1 | 0 | 1 | 1 | 0 | 1 | 0 | F | G | T | 621 | M − C₄O₂H₅ |
| 90 | 1 | 0 | 0 | 1 | 0 | 0 | 1 | 1 | 0 | G | C | T | 629 | M + H |
| 91 | 1 | 0 | 1 | 0 | 0 | 0 | 1 | 0 | 1 | F | D | U | 533 | M − C₄O₂H₅ |
| 92 | 0 | 0 | 1 | 0 | 1 | 0 | 1 | 0 | 1 | C | J | U | 511 | M + H |
| 93 | 0 | 0 | 1 | 0 | 0 | 1 | 0 | 1 | 0 | C | A | V | 436 | M − EtOH |
| 94 | 0 | 0 | 0 | 1 | 0 | 0 | 1 | 1 | 0 | D | C | V | 558 | M + H |
| 95 | 0 | 1 | 1 | 0 | 0 | 1 | 0 | 1 | 0 | H | A | W | 500 | M + H |
| 96 | 0 | 0 | 1 | 0 | 1 | 0 | 1 | 0 | 1 | C | J | W | 473 | M + H |
| 97 | 1 | 1 | 0 | 0 | 0 | 0 | 0 | 1 | 1 | E | E | X | 626 | M + H |
| 98 | 0 | 0 | 1 | 0 | 1 | 1 | 1 | 0 | 0 | C | F | X | 514 | M + H |
| 99 | 0 | 0 | 1 | 0 | 1 | 1 | 1 | 0 | 0 | C | F | Y | 365 | M + H |
| 100 | 0 | 0 | 0 | 1 | 1 | 0 | 1 | 1 | 0 | D | I | Y | 427 | M − EtOH |
| 101 | 1 | 0 | 0 | 0 | 1 | 0 | 0 | 0 | 0 | A | A | | 298 | M + Na |
| 102 | 1 | 0 | 1 | 0 | 1 | 0 | 0 | 0 | 1 | F | I | | 488 | M − EtOH |
| 103 | 0 | 0 | 0 | 1 | 1 | 1 | 0 | 0 | 1 | D | H | | 427 | M − EtOH |
| 104 | 0 | 1 | 0 | 0 | 0 | 1 | 0 | 0 | 1 | B | B | | 348 | M + H |
| 105 | 1 | 0 | 1 | 0 | 1 | 0 | 1 | 0 | 0 | F | G | | 420 | M − C₄O₂H₅ |
| 106 | 1 | 0 | 1 | 0 | 1 | 0 | 0 | 0 | 0 | F | A | | 346 | M + Na |
| 107 | 1 | 1 | 0 | 0 | 0 | 0 | 1 | 0 | 1 | E | D | | 438 | M − EtOH |
| 108 | 0 | 1 | 0 | 0 | 1 | 1 | 0 | 1 | 0 | B | G | | 348 | M + H |

### Example 5

Functionalization of Polystyrene-Grafted Lanterns with an Alkylsilane Linker

The scheme below illustrates functionalization of a polystyrene-grafted lantern (catalog # SP-PS-D-NOF SynPhase-Polystyrenc-D-size-Non-Functionalized) Mimotopes, Pty Ltd, Duerdin St., Clayton, Victoria 3168, Australia). The scheme illustrates the lantern and provides an expanded view of the polystyrene surface thereof. The first step involves functionalizing the polystyrene surface with bromine using a thallium acetate catalyst in a similar fashion to that described above. The reaction resulted in addition of between 40 and 60 micromole Br per lantern as determined by elemental analysis. Addition of the silicon-containing linker using Suzuki coupling as shown below resulted in between 40 and 60 micromoles linker/lantern as determined by elemental analysis and small molecule loading experiments such as FMOC quantitation.

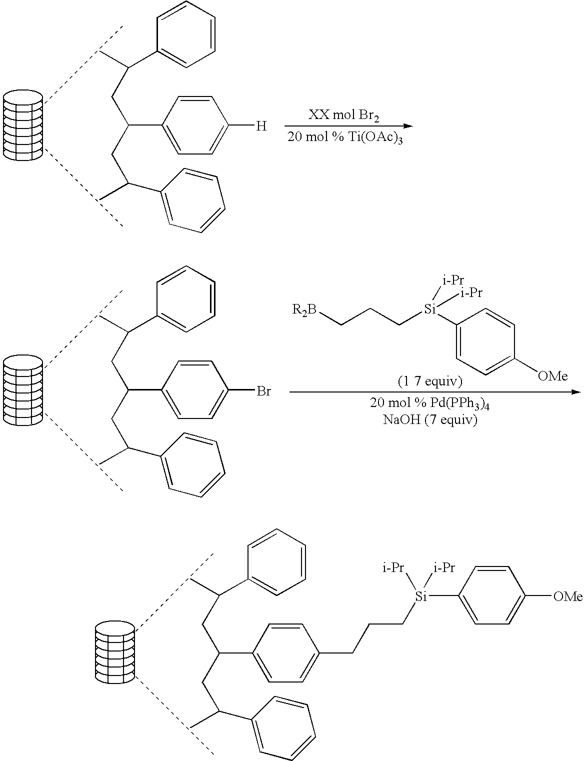

### Example 6

Biological Testing

Cell and Protein Based Screens

It will be appreciated that the small molecule compounds synthesized utilizing the solid supports and methods of the present invention may be screened in any of a variety of biological assays, for example, cell-based assays may be employed. Such cell-based assays generally involve contacting a cell with a compound and detecting any of a number of events, such as binding of the compound to the cell, initiation of a biochemical pathway or physiological change in the cell, changes in cell morphology, initiation or blockage of the cell cycle etc.

As but one example, once synthesized, the compounds may be arrayed in 384-well plates, by a robotic 384 pin arrayer, as shown in FIG. 10, and assayed for their ability to bind to a particular cell type present in the well. Detection can be carried out, for example, by detecting a tag that is attached to the small molecule. Alternatively, the small molecule may be detected by using a second molecule that has a tag, the second molecule specifically binding the small molecule, e.g., a tagged antibody specific to the small molecule.

Alternatively or additionally, inventive compounds may be studied in binding assays. In such assays, the compounds are bound to a solid support and then contacted with a protein of interest. The presence or absence of binding between the compound and the protein is then detected. In certain cases, the protein itself is tagged with a molecule that can be detected, e.g., with a fluorescent molecule. Alternatively, the protein is detected by utilizing any immunoassay, such as the ELISA.

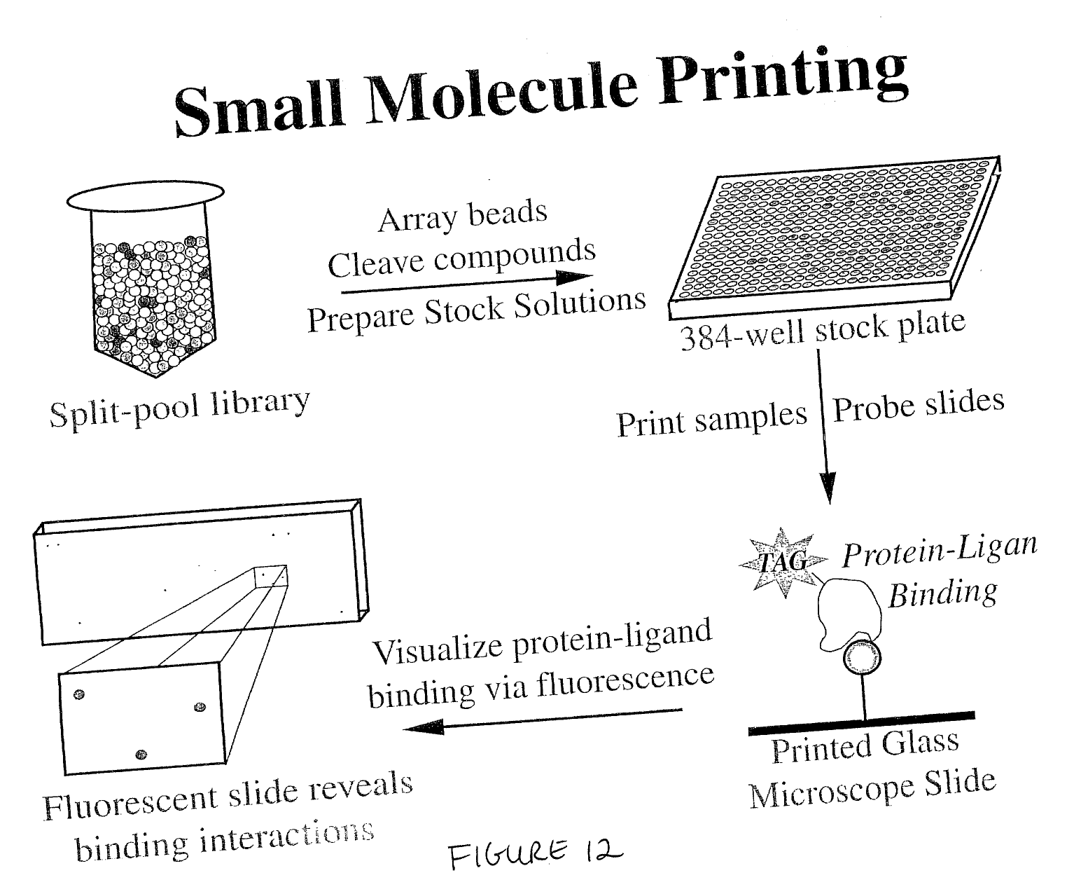

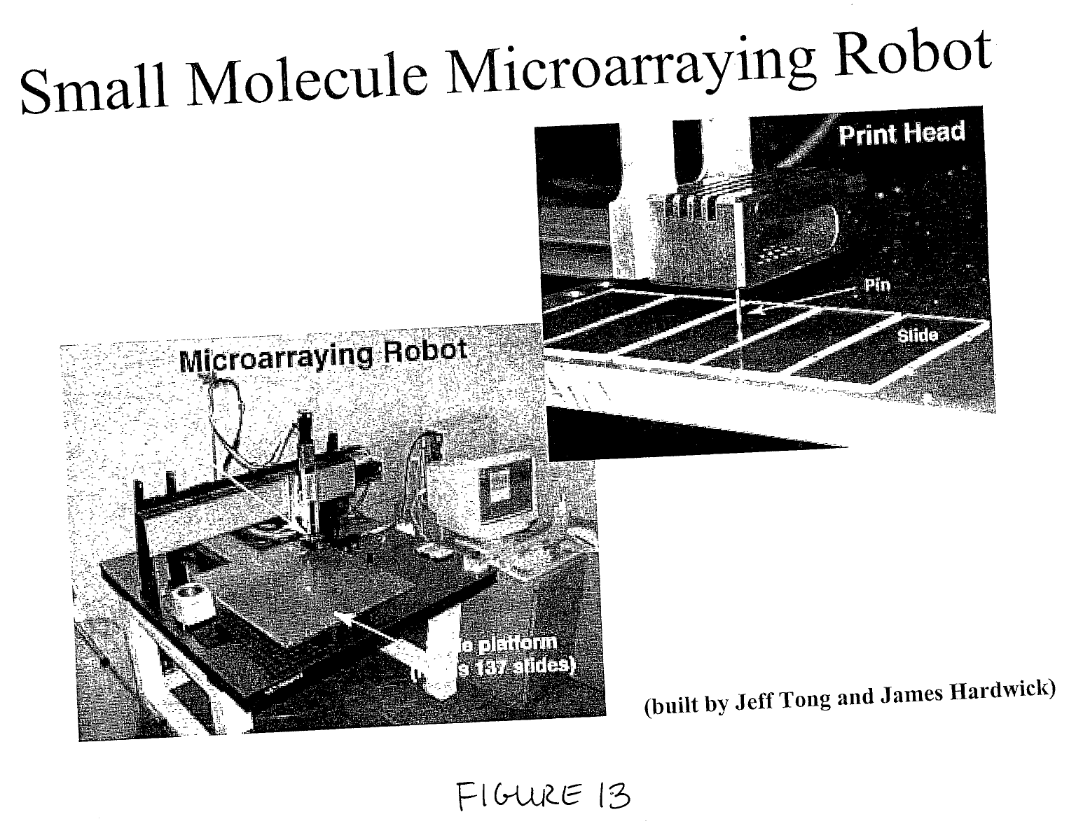

For example, a process known as small molecule printing (see, for example, U.S. Ser. No. 09/567,910, filed May 10, 2000, the entire contents of which are hereby incorporated by reference, may be utilized to screen proteins that interact with the library compounds. First, a split pool library is arrayed onto beads. The compounds are then cleaved from the beads and prepared in a standard stock solution, such as DMSO. The compounds are then arrayed onto a 384-well stock plate. Next, the compounds are printed onto glass slides, e.g., a glass microscope slides, and the slides are probed with a tagged ligand, e.g., a tagged protein of interest. Binding between a compound and the ligand is then detected by any available means appropriate to the tag being utilized, e.g., via fluorescence. (See FIGS. 12 and 13)

Exemplary biological assays that may be performed using compounds synthesized using the solid supports and methods of the invention include assays for molecules that affect protein trafficking, assays for compounds that enhance wound healing, assays for anti-microbial compounds, etc. These assays and screens are described in detail in copending application Ser. No. 09/863,141, entitled “Novel Alkaloids”. However, it is to be understood that compounds synthesized in accordance with the invention may be used in screens for any of a wide variety of biological and/or chemical activities.

We claim:

1. A solid support for solid-phase compound synthesis comprising:
a bead having the capacity to support synthesis of at least 50 nmol of compound, wherein the bead is functionalized with a silicon-containing linker.

2. The solid support of claim 1, wherein the silicon-containing linker has the structure (I): 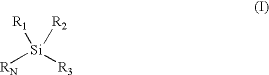
wherein R_{N}, is an aliphatic or heteroaliphatic moiety, wherein R_{N} is attached to the solid support;
R₁ and R₂ are each independently hydrogen or an aliphatic, heteroaliphatic, aryl or heteroaryl moiety; and
R₃ is an aliphatic, heteroaliphatic, aryl or heteroaryl moiety; halogen, —OSO₂Rₓ; wherein Rₓ is an aliphatic, heteroaliphatic, aryl or heteroaryl moiety; or O, S, —NR^{A} or —CR^{A}R^{B}; wherein any two adjacent —CR^{A}R^{B}; moieties may be linked by a single or double bond as valency permits; wherein each occurrence of R_{A} and R_{B} is independently absent, hydrogen, an aliphatic, heteroaliphatic, aryl or heteroaryl moiety, halogen, —CN, —S(O)ₘR^{J}, —NO₂, —COR^{J}, —CO₂R^{J}, —NR^{J}COR^{J}, —NR^{J}(CO)NR^{J}R^{J}, —NR^{J}CO₂R^{J}, —CONR^{J}R^{J}, —CO(NOR^{J})R^{J}, or —ZR^{J}, wherein Z is —O—, —S—, or NR^{K}; wherein each occurrence of R^{J} and R^{K} is independently hydrogen, —COR^{J}, —CO₂R^{J}, —CONR^{J}R^{J}, —CO(NOR^{J})R^{J}, or an aliphatic, heteroaliphatic, aryl or heteroaryl moiety, and m is 1 or 2;
wherein each of the foregoing aliphatic or heteroaliphatic moieties may be substituted or unsubstituted, cyclic or acyclic, linear or branched, saturated or unsaturated; and each of the foregoing aryl and heteroaryl moieties may be substituted or unsubstituted.

3. The solid support of claim 2, wherein R₁ and R₂ are each independently alkyl, heterolalkyl, aryl or heteroaryl; wherein each of the foregoing alkyl or heteroalkyl moieties may be substituted or unsubstituted, cyclic or acyclic, linear or branched, saturated or unsaturated.

4. The solid support of claim 2, wherein R₃ is aryl, heteroaryl, alkenyl, heteroalkenyl, halogen, or —OSO₂Rₓ; wherein Rₓ is an aliphatic, heteroaliphatic, aryl or heteroaryl moiety; wherein each of the foregoing alkyl, alkenyl, heteroalkenyl, moieties may be substituted or unsubstituted, cyclic or acyclic, linear or branched, saturated or unsaturated; and each of the foregoing aryl and heteroaryl moieties may be substituted or unsubstituted.

5. The solid support of claim 2, wherein R_{N} is covalently attached to the solid support.

6. The solid support of claim 2, wherein R_{N} is an aliphatic or heteroaliphatic moiety between 1 and 10 atoms in length.

7. The solid support of claim 2, wherein R_{N} is an aliphatic or heteroaliphatic moiety between 1 and 5 atoms in length.

8. The solid support of claim 2, wherein R_{N} is a linear hydrocarbon chain.

9. The solid support of claim 2, wherein R₁ and R₂ are each independently substituted or unsubstituted lower alkyl, lower heteroalkyl, aryl or heteroaryl.

10. The solid support of claim 2, wherein R_{N}, R₁, and R₂ do not contain heteroatoms.

11. The solid support of claim 2, wherein R_{N}, R₁, and R₂ do not contain double bonds.

12. The solid support of claim 2, wherein R₁ and R₂ are each independently methyl, ethyl, n-propyl, isopropyl, n-butyl, sec-butyl, isobutyl, tert-butyl or phenyl.

13. The solid support of claim 12, wherein R₁ and R₂ are each isopropyl.

14. The solid support of claim 1, wherein the silicon-containing linker has the structure (II): 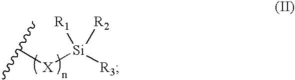
wherein R₁ and R₂ are each independently alkyl, heteroalkyl, aryl or heteroaryl;
R₃ is aryl, heteroaryl, alkenyl, heteroalkenyl, halogen, or —OSO₂Rₓ; wherein Rₓ is an aliphatic, heteroaliphatic, aryl or heteroaryl moiety;
X is O, S, —NR^{A} or —CR^{A}R^{B}; wherein any two adjacent —CR^{A}R^{B} moieties may be linked by a single or double bond as valency permits; wherein each occurrence of R_{A} and R_{B} is independently absent, hydrogen, an aliphatic, heteroaliphatic, aryl or heteroaryl moiety, halogen, —CN, —S(O)ₘR^{J}, —NO₂, —COR^{J}, —CO₂R^{J}, —NR^{J}COR^{J}, —NR^{J}(CO)NR^{J}R^{J}, —NR^{J}CO₂R^{J}, —CONR^{J}R^{J}, —CO(NOR^{J})R^{J}, or —ZR^{J}, wherein Z is —O—, —S—, or NR^{K}; wherein each occurrence of R^{J} and R^{K} is independently hydrogen, —COR^{J}, —CO₂R^{J}, —CONR^{J}R^{J}, —CO(NOR^{J})R^{J}, or an aliphatic, heteroaliphatic, aryl or heteroaryl moiety, and m is 1 or 2; and
n is an integer from 1-10;
wherein each of the foregoing alkyl, alkenyl, heteroalkenyl, heteroalkyl, aliphatic and heteroaliphatic moieties may be substituted or unsubstituted, cyclic or acyclic, linear or branched; and each of the foregoing aryl and heteroaryl moieties may be substituted or unsubstituted.

15. The solid support of claim 14, wherein X is —CR^{A}R^{B}; wherein any two adjacent —CR^{A}R^{B} moieties may be linked by a single or double bond as valency permits; wherein each occurrence of R_{A} and R_{B} is independently absent, hydrogen, an aliphatic, heteroaliphatic, aryl or heteroaryl moiety, halogen, —CN, —S(O)ₘR^{J}, —NO₂, —COR^{J}, —CO₂R^{J}, —NR^{J}COR^{J}, —NR^{J}(CO)NR^{J}R^{J}, —NR^{J}CO₂R^{J}, —CONR^{J}R^{J}, —CO(NOR^{J})R^{J}, or —ZR^{J}, wherein Z is —O—, —S—, or NR^{K}; wherein each occurrence of R^{J} and R^{K} is independently hydrogen, —COR^{J}, —CO₂R^{J}, —CONR^{J}R^{J}, —CO(NOR^{J})R^{J}, or an aliphatic, heteroaliphatic, aryl or heteroaryl moiety, and m is 1 or 2; and
n is an integer from 1-5;
wherein each of the foregoing aliphatic and heteroaliphatic moieties may be substituted or unsubstituted, cyclic or acyclic, linear or branched; and each of the foregoing aryl and heteroaryl moieties may be substituted or unsubstituted.

16. The solid support of claim 15, wherein X is —CR^{A}R^{B}; wherein each occurrence of R_{A} and R_{B} is independently hydrogen, lower alkyl, lower heteroalkyl, aryl, heteroaryl, halogen, —CN, —S(O)ₘR^{J}, —NO₂, —COR^{J}, —CO₂R^{J}, —NR^{J}COR^{J}, —NR^{J}(CO)NR^{J}R^{J}, —NR^{J}CO₂R^{J}, —CONR^{J}R^{J}, —CO(NOR^{J})R^{J}, or —ZR^{J}, wherein Z is —O—, —S—, or NR^{K}; wherein each occurrence of R^{J} and R^{K} is independently hydrogen, —COR^{J}, —CO₂R^{J}, —CONR^{J}R^{J}, —CO(NOR^{J})R^{J}, or an aliphatic, heteroaliphatic, aryl or heteroaryl moiety, and m is 1 or 2; and
n is an integer from 1-5;
wherein each of the foregoing alkyl, heteroalkyl, aliphatic and heteroaliphatic moieties may be substituted or unsubstituted, cyclic or acyclic, linear or branched; and each of the foregoing aryl and heteroaryl moieties may be substituted or unsubstituted.

17. The solid support of claim 16, wherein each occurrence of X is —CH₂; and
n is an integer from 1-5.

18. The solid support of claim 17, wherein each occurrence of X is —CH₂; and n is 3.

19. The solid support of claim 14, wherein R₁ and R₂ are each independently substituted or unsubstituted lower alkyl, lower heteroalkyl, aryl or heteroaryl.

20. The solid support of claim 19, wherein R₁ and R₂ are each independently methyl, ethyl, n-propyl, isopropyl, n-butyl, sec-butyl, isobutyl, tert-butyl or phenyl.

21. The solid support of claim 20, wherein R₁ and R₂ are each isopropyl.

22. The solid support of claim 14, wherein R₃ is aryl, heteroaryl, alkenyl, heteroalkenyl, halogen, or —OSO₂CF₃; wherein each of the foregoing alkenyl and heteroalkenyl moieties may be substituted or unsubstituted, cyclic or acyclic, linear or branched; and each of the foregoing aryl and heteroaryl moieties may be substituted or unsubstituted.

23. The solid support of claim 22, wherein R₃ is substituted aryl.

24. The solid support of claim 23, wherein R₃ is substituted phenyl.

25. The solid support of claim 24, wherein R₃ is phenyl substituted with one or more occurrences of halogen, lower alkyl or lower alkoxy.

26. The solid support of claim 25, wherein R₃ is a moiety having the structure: 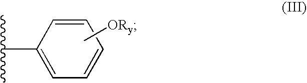
wherein R_{y} is lower alkyl.

27. The solid support of claim 14, wherein the silicon-containing linker has the structure:

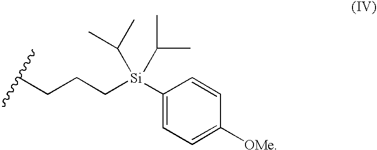

28. The solid support of claim 1, wherein the bead is formed from polystyrene.

29. The solid support of claim 1, wherein the bead has a diameter of at least approximately 400 μm

30. The solid support of claim 1, wherein the diameter of the bead is at least approximately 500 μm.

31. The solid support of claim 1, wherein the diameter of the bead is between approximately 500 and approximately 600 μm.

32. A solid support for compound synthesis comprising:
a polymeric surface grafted onto a rigid base polymer, wherein the polymeric surface is functionalized with a silicon-containing linker for attachment of a substrate on the solid support.

33. The solid support of claim 32, wherein the linker has the structure (I): 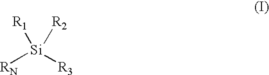
wherein R_{N}, is an aliphatic or heteroaliphatic moiety, wherein R_{N} is attached to the solid support;
R₁ and R₂ are each independently hydrogen or an aliphatic, heteroaliphatic, aryl or heteroaryl moiety; and
R₃ is an aliphatic, heteroaliphatic, aryl or heteroaryl moiety; halogen, —OSO₂Rₓ; wherein Rₓ is an aliphatic, heteroaliphatic, aryl or heteroaryl moiety; or O, S, —NR^{A} or —CR^{A}R^{B}; wherein any two adjacent —CR^{A}R^{B} moieties may be linked by a single or double bond as valency permits; wherein each occurrence of R_{A} and R_{B} is independently absent, hydrogen, an aliphatic, heteroaliphatic, aryl or heteroaryl moiety, halogen, —CN, —S(O)ₘR^{J}, —NO₂, —COR^{J}, —CO₂R^{J}, —NR^{J}COR^{J}, —NR^{J}(CO)NR^{J}R^{J}, —NR^{J}CO₂R^{J}, —CONR^{J}R^{J}, —CO(NOR^{J})R^{J}, or —ZR^{J}, wherein Z is —O—, —S—, or NR^{K}; wherein each occurrence of R^{J} and R^{K} is independently hydrogen, —COR^{J}, —CO₂R^{J}, —CONR^{J}R^{J}, —CO(NOR^{J})R^{J}, or an aliphatic, heteroaliphatic, aryl or heteroaryl moiety, and m is 1 or 2;
wherein each of the foregoing aliphatic or heteroaliphatic moieties may be substituted or unsubstituted, cyclic or acyclic, linear or branched, saturated or unsaturated; and each of the foregoing aryl and heteroaryl moieties may be substituted or unsubstituted.

34. The solid support of claim 33, wherein R₁ and R₂ are each independently alkyl, heterolalkyl, aryl or heteroaryl; wherein each of the foregoing alkyl or heteroalkyl moieties may be substituted or unsubstituted, cyclic or acyclic, linear or branched, saturated or unsaturated.

35. The solid support of claim 33, wherein R₃ is aryl, heteroaryl, alkenyl, heteroalkenyl, halogen, or —OSO₂Rₓ; wherein Rₓ is an aliphatic, heteroaliphatic, aryl or heteroaryl moiety; wherein each of the foregoing alkyl, alkenyl, heteroalkenyl, moieties may be substituted or unsubstituted, cyclic or acyclic, linear or branched, saturated or unsaturated; and each of the foregoing aryl and heteroaryl moieties may be substituted or unsubstituted.

36. The solid support of claim 33, wherein R_{N} is covalently attached to the solid support.

37. The solid support of claim 33, wherein R_{N} is an aliphatic or heteroaliphatic moiety between 1 and 10 atoms in length.

38. The solid support of claim 33, wherein R_{N} is an aliphatic or heteroaliphatic moiety between 1 and 5 atoms in length.

39. The solid support of claim 33, wherein R_{N} is a linear hydrocarbon chain.

40. The solid support of claim 33, wherein R₁ and R₂ are each independently substituted or unsubstituted lower alkyl, lower heteroalkyl, aryl or heteroaryl.

41. The solid support of claim 33, wherein R_{N}, R₁, and R₂ do not contain heteroatoms.

42. The solid support of claim 33, wherein R_{N}, R₁, and R₂ do not contain double bonds.

43. The solid support of claim 33, wherein R₁ and R₂ are each independently methyl, ethyl, n-propyl, isopropyl, n-butyl, sec-butyl, isobutyl, tert-butyl or phenyl.

44. The solid support of claim 43, wherein R₁ and R₂ are each isopropyl.

45. The solid support of claim 32, wherein the silicon-containing linker has the structure (II): 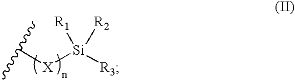
wherein R₁ and R₂ are each independently alkyl, heteroalkyl, aryl or heteroaryl;
R₃ is aryl, heteroaryl, alkenyl, heteroalkenyl, halogen, or —OSO₂Rₓ; wherein Rₓ is an aliphatic, heteroaliphatic, aryl or heteroaryl moiety;
X is O, S, —NR^{A} or —CR^{A}R^{B}; wherein any two adjacent —CR^{A}R^{B} moieties may be linked by a single or double bond as valency permits; wherein each occurrence of R_{A} and R_{B} is independently absent, hydrogen, an aliphatic, heteroaliphatic, aryl or heteroaryl moiety, halogen, —CN, —S(O)ₘR^{J}, —NO₂, —COR^{J}, —CO₂R^{J}, —NR^{J}COR^{J}, —NR^{J}(CO)NR^{J}R^{J}, —NR^{J}CO₂R^{J}, —CONR^{J}R^{J}, —CO(NOR^{J})R^{J}, or —ZR^{J}, wherein Z is —O—, —S—, or NR^{K}; wherein each occurrence of R^{J} and R^{K} is independently hydrogen, —COR^{J}, —CO₂R^{J}, —CONR^{J}R^{J}, —CO(NOR^{J})R^{J}, or an aliphatic, heteroaliphatic, aryl or heteroaryl moiety, and m is 1 or 2; and
n is an integer from 1-10;
wherein each of the foregoing alkyl, alkenyl, heteroalkenyl, heteroalkyl, aliphatic and heteroaliphatic moieties may be substituted or unsubstituted, cyclic or acyclic, linear or branched; and each of the foregoing aryl and heteroaryl moieties may be substituted or unsubstituted.

46. The solid support of claim 45, wherein X is —CR^{A}R^{B}; wherein any two adjacent —CR^{A}R^{B} moieties may be linked by a single or double bond as valency permits; wherein each occurrence of R_{A} and R_{B} is independently absent, hydrogen, an aliphatic, heteroaliphatic, aryl or heteroaryl moiety, halogen, —CN, —S(O)ₘR^{J}, —NO₂, —COR^{J}, —CO₂R^{J}, —NR^{J}COR^{J}, —NR^{J}(CO)NR^{J}R^{J}, —NR^{J}CO₂R^{J}, —CONR^{J}R^{J}, —CO(NOR^{J})R^{J}, or —ZR^{J}, wherein Z is —O—, —S—, or NR^{K}; wherein each occurrence of R^{J} and R^{K} is independently hydrogen, —COR^{J}, —CO₂R^{J}, —CONR^{J}R^{J}, —CO(NOR^{J})R^{J}, or an aliphatic, heteroaliphatic, aryl or heteroaryl moiety, and m is 1 or 2; and
n is an integer from 1-5;
wherein each of the foregoing aliphatic and heteroaliphatic moieties may be substituted or unsubstituted, cyclic or acyclic, linear or branched; and each of the foregoing aryl and heteroaryl moieties may be substituted or unsubstituted.

47. The solid support of claim 46, wherein X is —CR^{A}R^{B}; wherein each occurrence of R_{A} and R_{B} is independently hydrogen, lower alkyl, lower heteroalkyl, aryl, heteroaryl, halogen, —CN, —S(O)ₘR^{J}, —NO₂, —COR^{J}, —CO₂R^{J}, —NR^{J}COR^{J}, —NR^{J}(CO)NR^{J}R^{J}, —NR^{J}CO₂R^{J}, —CONR^{J}R^{J}, —CO(NOR^{J})R^{J}, or —ZR^{J}, wherein Z is —O—, —S—, or NR^{K}; wherein each occurrence of R^{J}and R^{K} is independently hydrogen, —COR^{J}, —CO₂R^{J}, —CONR^{J}R^{J}, —CO(NOR^{J})R^{J}, or an aliphatic, heteroaliphatic, aryl or heteroaryl moiety, and m is 1 or 2; and
n is an integer from 1-5;
wherein each of the foregoing alkyl, heteroalkyl, aliphatic and heteroaliphatic moieties may be substituted or unsubstituted, cyclic or acyclic, linear or branched; and each of the foregoing aryl and heteroaryl moieties may be substituted or unsubstituted.

48. The solid support of claim 47, wherein each occurrence of X is —CH₂; and
n is an integer from 1-5.

49. The solid support of claim 48, wherein each occurrence of X is —CH₂; and n is 3.

50. The solid support of claim 45, wherein R₁ and R₂ are each independently substituted or unsubstituted lower alkyl, lower heteroalkyl, aryl or heteroaryl.

51. The solid support of claim 50, wherein R₁ and R₂ are each independently methyl, ethyl, n-propyl, isopropyl, n-butyl, sec-butyl, isobutyl, tert-butyl or phenyl.

52. The solid support of claim 51, wherein R₁ and R₂ are each isopropyl.

53. The solid support of claim 45, wherein R₃ is aryl, heteroaryl, alkenyl, heteroalkenyl, halogen, or —OSO₂CF₃; wherein each of the foregoing alkenyl and heteroalkenyl moieties may be substituted or unsubstituted, cyclic or acyclic, linear or branched; and each of the foregoing aryl and heteroaryl moieties may be substituted or unsubstituted.

54. The solid support of claim 53, wherein R₃ is substituted aryl.

55. The solid support of claim 54, wherein R₃ is substituted phenyl.

56. The solid support of claim 55, wherein R₃ is phenyl substituted with one or more occurrences of halogen, lower alkyl or lower alkoxy.

57. The solid support of claim 56, wherein R₃ is a moiety having the structure: 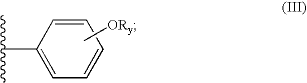
wherein R_{y} is lower alkyl.

58. The solid support of claim 45, wherein the silicon-containing linker has the structure:

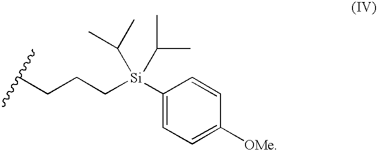

59. The solid support of claim 45, wherein the solid support may be activated prior to attachment of the substrate to the solid support.

60. The solid support of claim 45, wherein the silicon-containing linker has the structure: 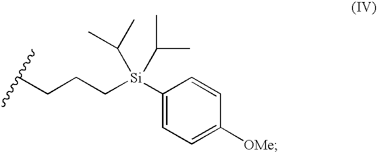
and activation of the solid support comprises treating the functionalized solid support with triflic acid, thereby generating a solid support functionalized with a silicon-containing linker having the structure:

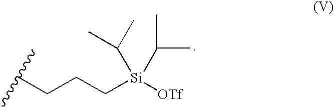

61. The solid support of claim 32, wherein the polymeric surface comprises polystyrene.

62. The solid support of claim 32, wherein the base polymer is a copolymer of polyethylene and polypropylene.

63. The solid support of claim 32, wherein the rigid base polymer assumes a three-dimensional form.

64. The solid support of claim 63, wherein the three-dimensional form is a lantern, crown, gear, pin, puck, disc, bead, microtitre plate or sheet.

65. The solid support of claim 64, wherein the three-dimensional form is a lantern.

66. A method of performing solid-phase synthesis of a compound comprising steps of:
(i) providing a solid support having a capacity to support synthesis of at least 50 nmol of compound;
(ii) attaching a linker to the solid support;
(iii) reacting a substrate with the linker, thereby loading the substrate onto the solid support; and
(iv) treating the support-bound substrate with a suitable reagent under suitable conditions to effect a desired chemical transformation; and
(v) optionally repeating step (iv) until desired functionalization of the substrate is achieved, thereby forming a support-bound target compound.

67. The method of claim 66, wherein the linker is a silicon-containing linker.

68. The method of claim 67, wherein the silicon-containing linker has the structure (I): 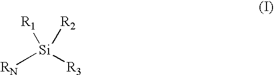
wherein R_{N}, is an aliphatic or heteroaliphatic moiety, wherein R_{N} is attached to the solid support;
R₁ and R₂ are each independently hydrogen or an aliphatic, heteroaliphatic, aryl or heteroaryl moiety; and
R₃ is an aliphatic, heteroaliphatic, aryl or heteroaryl moiety; halogen, —OSO₂Rₓ; wherein Rₓ is an aliphatic, heteroaliphatic, aryl or heteroaryl moiety; or O, S, —NR^{A} or —CR^{A}R^{B}; wherein any two adjacent —CR^{A}R^{B} moieties may be linked by a single or double bond as valency permits; wherein each occurrence of R_{A} and R_{B} is independently absent, hydrogen, an aliphatic, heteroaliphatic, aryl or heteroaryl moiety, halogen, —CN, —S(O)ₘR^{J}, —NO₂, —COR^{J}, —CO₂R^{J}, —NR^{J}COR^{J}, —NR^{J}(CO)NR^{J}R^{J}, —NR^{J}CO₂R^{J}, —CONR^{J}R^{J}, —CO(NOR^{J})R^{J}, or —ZR^{J}, wherein Z is —O—, —S—, or NR^{K}; wherein each occurrence of R^{J} and R^{K} is independently hydrogen, —COR^{J}, —CO₂R^{J}, —CONR^{J}R^{J}, —CO(NOR^{J})R^{J}, or an aliphatic, heteroaliphatic, aryl or heteroaryl moiety, and m is 1 or 2;
wherein each of the foregoing aliphatic or heteroaliphatic moieties may be substituted or unsubstituted, cyclic or acyclic, linear or branched, saturated or unsaturated; and each of the foregoing aryl and heteroaryl moieties may be substituted or unsubstituted.

69. The method of claim 68, wherein the silicon-containing linker has the structure:

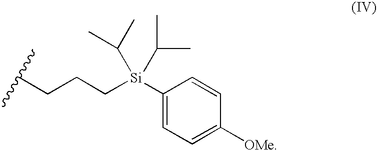

70. The method of claim 68, wherein the solid support may be activated prior to attachment of the substrate to the solid support.

71. The method of claim 70, wherein the silicon-containing linker has the structure: 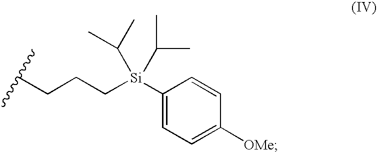
and activation of the solid support comprises treating the functionalized solid support with triflic acid, thereby generating a solid support functionalized with a silicon-containing linker having the structure:

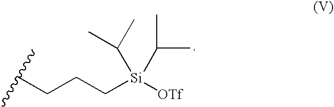

72. The method of claim 66, wherein the substrate comprises a hydroxyl group.

73. The method of claim 72, wherein the substrate is a primary, secondary or tertiary alcohol.

74. The method of claim 66, wherein the solid support is a bead.

75. The method of claim 74, wherein the bead has a diameter of at least 400 μm.

76. The method of claim 74, wherein the bead has a diameter of at least 500 μm.

77. The method of claim 74, wherein the bead has a diameter of between 500 and 600 μm.

78. The method of claim 66, wherein the solid support is a grafted polymeric support.

79. The method of claim 78, wherein the grafted polymeric support is shaped as a lantern.

80. A collection of compounds synthesized according to the method of claim 66.

81. The collection of claim 80, wherein the collection comprises at least 100 compounds.

82. The collection of claim 80, wherein the collection comprises at least 1,000 compounds.

83. The collection of claim 80, wherein the collection comprises at least 2,000 compounds.

84. The collection of claim 80, wherein the collection comprises at least 10,000 compounds.

85. The method of claim 66, further comprising a step of:
(vi) activating the linker prior to reacting the substrate with the linker.

86. The method of claim 85, the step of activating comprises treating the functionalized solid support with triflic acid.

87. A method of producing a library of compounds comprising:
performing the method of claim 66 using a plurality of solid supports, a plurality of substrates, and plurality of suuitable reagents.

88. A collection of compounds synthesized according to the method of claim 87.

89. The collection of claim 88, wherein the collection comprises at least 100 compounds.

90. The collection of claim 88, wherein the collection comprises at least 1,000 compounds.

91. The collection of claim 88, wherein the collection comprises at least 2,000 compounds.

92. The collection of claim 88, wherein the collection comprises at least 10,000 compounds.

93. The method of claim 66, further comprising a step of:
encoding step (iii), step (iv) and each of steps (v).

94. The method of claim 93, wherein the step of encoding comprises:
labeling the solid support, the substrate, the target compound or any combination of the foregoing with one or more chemical tags prior to or subsequent to performing each of steps (iii), (iv) and (v), wherein the tags are characteristic of the substrate identity and the reaction sequence performed in steps (iv) and (v).

95. The method of claim 94, wherein the chemical tag is a chloroaromatic diazoketone tag.

96. The method of claim 66, further comprising one or more of the following steps:
(vi) dispensing the solid support into a vessel;
(vii) cleaving the target compound from the solid support;
(vii) dissolving the cleaved compound in a suitable solvent; and
(ix) screening the compound for a biological or chemical activity.

97. The method of claim 96, wherein at least one of the steps of dispensing, cleaving, dissolving, or screening is performed robotically.

98. The method of claim 96, wherein the screening step includes a step of printing the compound onto a surface.

99. The method of claim 96, wherein the screening step includes a step of exposing a biological system to the compound and detecting a response.

100. The method of claim 96, wherein the identity of, and the reaction sequence leading to the target compound is encoded by labeling the solid support, the substrate, the target compound, or any combination of the foregoing with one or more chemical tags, further comprising a step of:
determining the reaction sequence by identifying the tags bound to the solid support.

101. The method of claim 96, wherein the vessel is a well of a microtiter plate.

102. A method of screening a compound library comprising steps of:
(i) arraying a plurality of support-bound target compounds prepared according to the method of claim 66 into a plurality of individual vessels;
(ii) cleaving the target compounds from the solid supports;
(iii) dissolving the cleaved target compounds in a solvent; and
(iv) screening the compounds for biological or chemical activity.

103. The method of claim 102, wherein each vessel contains a single support-bound target compound.

104. The method of claim 102, wherein the individual vessels are wells of a microtiter plate.

105. The method of claim 102, wherein at least one of the steps of dispensing, cleaving, dissolving, or screening is performed robotically.

106. The method of claim 102, wherein the screening step includes a step of printing the compound onto a surface.

107. The method of claim 102, wherein the screening step includes a step of exposing a biological system to the compound and detecting a response.

108. The method of claim 102, wherein the identity of, and the reaction sequence leading to the target compound is encoded by labeling the solid support, the substrate, the target compound, or any combination of the foregoing with one or more chemical tags, further comprising a step of:
determining the reaction sequence by identifying the tags bound to the solid support.

109. A method for encoding and decoding the reaction sequence leading to synthesis of one or more members of a compound library comprising:
performing sequences of chemical reactions resulting in the formation of target compounds, wherein each chemical reaction is encoded by incorporation of a chemical tag into the compound being synthesized; and
determining the reaction sequence of a synthesized compound by identifying the tags inserted into the compound.

110. The method of claim 109, wherein the sequence of chemical reactions is performed as a solid-phase synthesis.

111. The method of claim 110, further comprising a step of:
cleaving the compound from the solid support prior to identification of the tags.

112. The method of claim 109, wherein the tags are chloroaromatic diazoketone tags.

113. The linker of any of claims 14, 15, 16, 17, 18, 19, 22, 22, 24, 25, or 26, wherein R₁ and R₂ are each independently ethyl, n-propyl, isopropyl, n-butyl, sec-butyl, isobutyl, tert-butyl or phenyl.

114. The linker of any of claims 14, 15, 16, 17, 18, 19, 22, 23, 24, 25, or 25, wherein R₁ and R₂ are isopropyl.

115. The linker of claim 27.
